(19) United States
(12) Patent Application Publication
(10) Pub. No.: US 2010/0168167 A1
(43) Pub. Date: Jul. 1, 2010

(54) Piperidinones Useful in the Treatment of Inflammation

(76) Inventors: Benjamin Pelcman, Solna (SE); Christian Krog-Jensen, Solna (SE); Yaping Shen, Port Coquitlam (CA); James Gee Kan Yee, Vancouver (CA); Lloyd F. MacKenzie, Surrey (CA); Yuanlin Zhou, Richmond (CA); Kang Han, Vancouver (CA); Jeffery R. Raymond, Vancouver (CA)

Correspondence Address: K&L Gates LLP, 1900 MAIN STREET, SUITE 600, IRVINE, CA, 92614-7319, US

(21) Appl. No.: 12/530,578

(22) PCT Filed: Mar. 12, 2008
(86) PCT No.: PCT/GB2008/000854

§ 371 Date: Mar. 17, 2010

Related U.S. Application Data
(60) Provisional application No. 60/906,804, filed on Mar. 12, 2007.
(60) Provisional application No. 60/943,520, filed on Jun. 12, 2007.

(30) Foreign Application Priority Data
Nov. 21, 2007 (GB) 0722769.7

(51) Int. Cl. A61K 31/4545; A61P 11/00; A61P 1/00; A61P 19/02; A61P 19/08; A61P 7/12; A61P 35/04; C07D 211/40; A61K 31/445; C07D 417/04; A61K 31/454; C07D 401/02

(52) U.S. Cl. 514/318; 546/216; 514/327; 546/198; 514/321; 514/322; 546/199; 546/194

(57) ABSTRACT

There is provided compounds of formula (I): wherein R¹, R², R³, R⁴, R⁵, R⁶, R⁷, m and n have meanings given in the description, and pharmaceutically acceptable derivatives thereof, which compounds are useful in the treatment of diseases and conditions associated with inflammation.

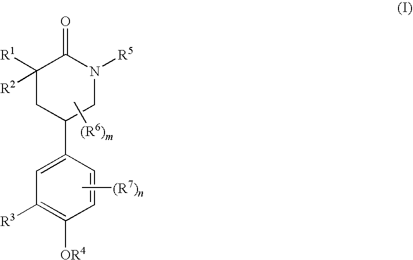

### FIELD OF THE INVENTION

The present invention is directed to substituted lactam compounds and their uses as therapeutic agents.

### BACKGROUND OF THE INVENTION

### The Inflammatory Response (Inflammation)

Inflammation is an essential localized host response to invading microorganisms or tissue injury which involves cells of the immune system. The classic signs of inflammation include redness (erythema), swelling (edema), pain and increased heat production (pyrema) at the site of injury. The inflammatory response allows the body to specifically recognize and eliminate an invading organism and/or repair tissue injury. Many of the acute changes at the site of inflammation are either directly or indirectly attributable to the massive influx of leukocytes (e.g., neutrophils, eosinophils, lymphocytes, monocytes) which is intrinsic to this response. Leukocytic infiltration and accumulation in tissue results in their activation and subsequent release of inflammatory mediators such as LTB₄, prostaglandins, TNF-α, IL-1β, IL-8, IL-5, IL-6, histamine, proteases and reactive oxygen species for example.

Normal inflammation is a highly regulated process that is tightly controlled at several levels for each of the cell types involved in the response. For example, expression of the pro-inflammatory cytokine TNF-α is controlled at the level of gene expression, translation, post-translational modification and release of the mature form from the cell membrane. Many of the proteins up-regulated during inflammation are controlled by the transcription factor, NF-KB. Pro-inflammatory responses are normally countered by endogenous anti-inflammatory mechanisms such as generation of IL-10 or IL-4. A characteristic of a normal inflammatory response is that it is temporary in nature and is followed by a resolution phase which brings the state of the tissue back to its prior condition. The resolution phase is thought to involve up-regulation of anti-inflammatory mechanisms, such as IL-10, as well as down-regulation of the proinflammatory processes.

### Inflammatory Disease

Inflammatory disease occurs when an inflammatory response is initiated that is inappropriate and/or does not resolve in the normal manner but rather persists and results in a chronic inflammatory state. Inflammatory disease may be systemic (e.g. lupus) or localized to particular tissues or organs and exerts an enormous personal and economic burden on society. Examples of some of the most common and problematic inflammatory diseases are rheumatoid arthritis, inflammatory bowel disease, psoriasis, asthma, chronic obstructive pulmonary disease, emphysema, colitis and ischemia-reperfusion injury.

A common underlying theme in inflammatory disease is a perturbation of the cellular immune response that results in recognition of host proteins (antigens) as foreign. Thus the inflammatory response becomes misdirected at host tissues with effector cells targeting specific organs or tissues often resulting in irreversible damage. The self-recognition aspect of auto-immune disease is often reflected by the clonal expansion of T-cell subsets characterized by a particular T-cell receptor (TCR) subtype in the disease state. Often inflammatory disease is also characterized by an imbalance in the levels of T-helper (Th) subsets (i.e., Th1 cells vs. Th2 cells).

Therapeutic strategies aimed at curing inflammatory diseases usually fall into one of two categories: (a) down-modulation of processes that are up-regulated in the disease state or (b) up-regulation of anti-inflammatory pathways in the affected cells or tissues. Most regimes currently employed in the clinic fall into the first category. Some examples of which are corticosteroids and non-steroidal anti-inflammatory drugs (NSAIDs).

Many of the tissue, cellular and biochemical processes which are perturbed in inflammatory disease have been elucidated and this has allowed the development of experimental models or assays to mimic the disease state. These in-vitro assays

enable selection and screening of compounds with a high probability of therapeutic efficacy in the relevant inflammatory disease. Thus, currently employed assays used to model the importance of the activated leukocytes in the development of acute inflammation and maintenance of the chronic inflammatory state are assays monitoring leukocyte chemotaxis and cellular degranulation and cytokine synthesis and reactive oxygen species (ROS) production assays in vitro. Since a result of acute or chronic neutrophil activation is release of ROS with resultant tissue damage, an assay for scavengers of ROS allows detection of compounds with potential therapeutic efficacy.

Cellular assays to detect inhibitors of TNF-α release from stimulated macrophage or monocytic cells are an important component of an in vitro model for inflammation as this cytokine is upregulated and has been shown to contribute to the pathology in many inflammatory diseases. Since elevated cAMP in affected cells has been shown to modulate or dampen the inflammatory response, monitoring cellular cyclic AMP (cAMP) levels, and the activity of pathways controlling cAMP levels allows for the detection of potential anti-inflammatory compounds. Assays may include monitoring the level of cAMP itself, phosphodiesterase activity, or changes in cAMP response element (CRE)-luciferase activity.

### Cyclic Nucleotide Messengers and Phosphodiesterases

The cyclic nucleotides, cyclic adenosine monophosphate (cAMP) and cyclic guanosine monophosphate (cGMP), play a key role in regulating cell function and phosphodiesterases (PDEs) provide the main route for the degradation of cyclic nucleotides. cAMP is now known to control the functional and genomic responses for a variety of cellular functions triggered by a wide array of receptors (Beavo, J. A. and Brunton, L. L., Nat. Rev. Mol. Cell. Biol., 3, 710-718 (2002)). Local control of cAMP signalling is affected by a complex pattern of localized synthesis, by adenylate cyclise (AC), and by phosphodiesterase (PDE)-mediated enzymatic degradation.

The PDEs are a family of enzymes that catalyze the hydrolysis of 3′,5′-cyclic nucleotides to 5′ nucleoside monophosphates, including the conversion of cAMP to AMP and cGMP to GMP. PDE enzymes are collectively grouped as a superfamily of eleven different, but homologous, gene-families with a highly conserved catalytic domain (Soderling, S. H. and Beavo, J. A., Curr. Opin. Cell Biol., 12, 174-179 (2000)). At present twenty-one different mammalian PDE genes have been identified. Many of these genes are expressed in multiple isoforms either by differing initiation sequences or splicing patterns. Differentiation of the enzymes can be achieved on the basis of substrate specificity, kinetic properties and sensitivity to regulatory molecules. PDEs in families 5, 6 and 9 specifically catalyze the hydrolysis of cGMP while PDEs 4, 7 and 8 are specific for cAMP. Enzymes belonging to the other PDE families (1, 2, 3, 10 and 11) catalyze the hydrolysis of both cAMP and cGMP with differing kinetics. Different PDE isozymes can have specific tissue, cellular and subcellular distributions and more than one type of PDE is usually present in any given cell. The types of PDEs expressed in a cell, together with their relative proportions and subcellular localization, control the cyclic nucleotide phenotype of that cell.

The PDE4 enzyme is responsible for selective, high affinity hydrolytic degradation of the second messenger cAMP, has a low Michaelis constant and is sensitive to inhibition by rolipram. The PDE4 enzyme family consists of four genes, which produce 4 isoforms of the PDE4 enzyme (PDE4A, PDE4B, PDE4C, and PDE4D) (Wang et al., “Expression, Purification, and Characterization of human cAMP Specific Phosphodiesterase (PDE4) Subtypes A, B, C, and D, Biochem”, Biophys. Res. Comm., 234, 320-324 (1997)). Moreover, various splice variants of each PDE4 isoform have been identified and play a role in the compartmentalized cAMP signalling in cells (Houslay, M. D., Schafer, P., and Zhang, K. Y., Drug Discov. Today, 15; 10(22):1503-19 (2005)). Recently, a number of selective PDE4 inhibitors have been discovered to have beneficial pharmacological effects resulting from PDE4 inhibition as shown in a variety of disease models (Torphy et al., Environ. Health Perspect., 102 Suppl. 10, 79-84, 1994; Duplantier et al., J. Med. Chem., 39 120-125 (1996); Schneider et al., Pharmacol. Biochem. Behay., 50, 211-217 (1995); Banner and Page, Br. J. Pharmacol., 114, 93-98 (1995); Barnette et al., J. Pharmacol. Exp. Ther., 273, 674-679 (1995); Wright et al., “Differential in vivo and in vitro bronchorelaxant activities of CP-80633, a selective phosphodiesterase 4 inhibitor,” Can. J. Physiol. Pharmacol., 75, 1001-1008 (1997); Manabe et al., “Anti-inflammatory and bronchodilator properties of KF19514, a phosphodiesterase 4 and 1 inhibitor,” Eur. J. Pharmacol., 332, 97-107 (1997); and Ukita et al., “Novel, potent, and selective phosphodiesterase-4 inhibitors as antiasthmatic agents: synthesis and biological activities of a series of 1-pyridylnaphthalene derivatives,” J. Med. Chem., 42, 1088-1099 (1999)). Therefore, considerable interest exists in the discovery of additional selective inhibitors of PDE4.

Regulation of cAMP activity is important in many biological processes, including inflammation, depression and cognitive function. Chronic inflammation is a multitude of heterogeneous diseases characterized in part by activation of multiple inflammatory cells, particularly cells of lymphoid lineage (including T lymphocytes) and myeloid lineage (including granulocytes, macrophages, and monocytes). Activation of these inflammatory cells results in production and release of proinflammatory mediators, including cytokines and chemokines, such as tumor necrosis factor (TNF) and interleukin-1 (IL-1). Discovery of a molecule that suppresses or inhibits such cellular activation and proinflammatory mediator release would be useful in the therapeutic treatment of inflammatory diseases. Elevated cAMP levels suppress inflammatory cell activation. Increased cAMP levels associated with PDE4 inhibition has therefore become a valid potential therapeutic approach to control inflammatory responses and disorders (Beavo et al., “Cyclic Nucleotide Phosphodiesterases: Structure, Regulation and Drug Action,” Wiley and Sons, Chichester, pp. 3-14 (1990); Torphy et al., Drug News and Perspectives, 6, pp. 203-214 (1993); Giembycz et al., Clin. Exp. Allergy, 22, pp. 337-344 (1992); and Sanz, M. J., Cortijo, J., Morcillo, E. J., Pharmacol Ther. 106(3):269-97 (2005)).

PDE4 inhibitors have recently shown clinical utility in mitigating the effects of the chronic pulmonary inflammatory diseases of asthma and chronic obstructive pulmonary disease (COPD). Roflumilast, a selective PDE4 inhibitor, demonstrated improvements in measures of airway function (forced expiratory volume in 1 second; FEV1, and peak expiratory flow; PEF) in mild asthmatics in a recently published clinical trial of 12 weeks duration (Bateman et al., Ann. Allergy Asthma Immunol., 96(5): 679-86 (2006)). A separate study with roflumilast also demonstrated improvements in airway hyper-responsiveness (AHR) to direct histamine provocation in a similar group of mild asthmatics in response to allergen challenge (Louw et al., Respiration, Sep. 5, 2006). Recently published results of a long term (6 month) study of cilomilast treatment in patients with COPD indicated that treatment with a selective PDE4 inhibitor arrested airway function (FEV1) decline in these patients and positively affected their quality of life as measured by the St. Georges Respiratory Questionnaire (Rennard et al., Chest, 129(1) 65-66 (2006)).

The clinical usefulness of PDE4 inhibition has also been demonstrated in disorders of the central nervous system. PDE4 inhibition by rolipram improves cognitive function in rodents and was developed as an antidepressant in humans. cAMP acts as a second messenger for neurotransmitters, and thus mediates their cellular responses. The therapeutic effects of PDE4 inhibitors in cognition and depression likely originate from enhancement of the cAMP-dependent cellular responses.

The listing or discussion of an apparently prior-published document in this specification should not necessarily be taken as an acknowledgement that the document is part of the state of the art or is common general knowledge.

International patent application WO 2007/081570 discloses various compounds that may be useful in the treatment of cholesterol-related diseases. However, there is no disclosure that such compounds may be useful as phosphodiesterase 4 inhibitors, and therefore in the treatment of inflammation. Further, there is no specific disclosure of piperidin-2-ones that are substituted at the 3-position.

International patent application WO 2006/124874 discloses a broad range of inter alia heterocyclic compounds that may be of use as inhibitors of B-Raf, and therefore of use in the treatment of cancer. There is no specific disclosure in that document of piperidin-2-ones substituted with a phenyl group at the 5-position.

US patents/applications U.S. Pat. No. 6,162,927, US 2002/0055457 and U.S. Pat. No. 7,208,517 and international patent applications WO 2002/11713, WO 2002/011713, WO 99/006397, WO 96/006095, WO 97/030045 and WO 02/017912 all disclose various compounds that may be useful as endothelin antagonists, and therefore of use in the treatment of cancer. There is no specific disclosure in any of these documents of piperidin-2-ones that are substituted at the 5-position with a phenyl group.

US patent application US 2007/0203124 discloses various piperazines that may be useful as inhibitors of phosphodiesterase 4 function. However, there is no disclosure in that document of piperidinones.

International patent application WO 01/68600 discloses various compounds, including pyrrolidinones, that may be useful in the treatment of inflammation-based diseases. However, there is no disclosure in this document of compounds containing a core piperidin-2-one ring.

International patent application WO 2005/115389 discloses various compounds that may be useful in the treatment of negative energy balance in ruminants. However, there is no mention that the compounds disclosed therein may be useful in the treatment of inflammation.

International patent application WO 95/028926 discloses various heterocycles including pyrrolidinones and piperidinones as potentially useful phosphodiesterase 4 inhibitors. However, there is no specific disclosure in that document of piperidin-2-ones substituted in the 5-position with a phenyl ring.

Further, US patent application US 2003/0186943 and international patent applications WO 00/14083 and WO 2004/031149 disclose inter alia piperidin-2-ones that may be useful in the treatment of inflammation-based diseases. International patent applications WO 2007/137181, WO 2004/091609 and WO 2004/016227 and US application US 2004/0224316 disclose various piperidinones, which may be useful as phosphodiesterase 4 inhibitors. However, these documents only disclose certain 3-benzyl-5-phenylpiperidin-2-ones, as well as certain corresponding 3-unsubstituted-5-phenylpiperidin-2-one intermediates.

Also, European patent EP 299 549 discloses various piperidine derivatives which may have opiate-antagonistic activity. However, there is no mention that such compounds may be useful as phosphodiesterase 4 inhibitors, and therefore of use in the treatment of inflammation.

### DISCLOSURE OF THE INVENTION

According to the invention, there is now provided a compound of formula (I),

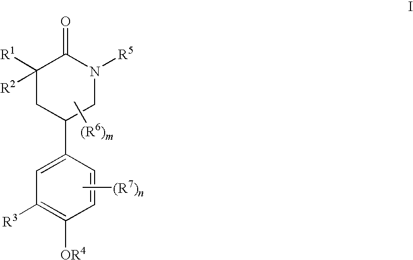

wherein:
m represents 0, 1, 2, 3, 4 or 5;
n represents 0, 1, 2 or 3;
R¹ represents hydrogen, C₁₋₁₂ alkyl, C₂₋₁₂ alkenyl, C₂₋₁₂ alkynyl (which latter three groups are optionally substituted by one or more substituents selected from X¹), -A¹-T^{z}-B¹, -A^{1a}-N(R⁹)R¹⁰, -A^{1b}-OR⁹, -A^{1c}-C(O)R⁹, -A^{1d}-C(O)R⁹ or -A^{1e}-C(O)N(R⁹)R¹⁰;
R² represents hydrogen, —OR⁴, C₁₋₁₂ alkyl, C₂₋₁₂ alkenyl or C₂₋₁₂ alkynyl, which latter three groups are optionally substituted by one or more substituents selected from X¹; or
R¹ and R² together form ═C(R⁹)R¹⁰;
R³ represents hydrogen, —OR⁴, C₁₋₁₂ alkyl, C₂₋₁₂ alkenyl, C₂₋₁₂ alkynyl (which latter three groups are optionally substituted by one or more substituents selected from X²) or -A²-B²;
each R⁴ independently represents, on each occasion when used herein, hydrogen, —R⁸—OR⁹, —R⁸—C(O)OR⁹, C₁₋₁₂ alkyl, C₂₋₁₂ alkenyl, C₂₋₁₂ alkynyl (which latter three groups are optionally substituted by one or more substituents selected from X³) and/or -A³-B³;
R⁵ represents hydrogen, -A⁴-B⁴, —C(O)R⁹, —C(O)OR¹⁰, C₁₋₁₂ alkyl, C₂₋₁₂ alkenyl or C₂₋₁₂ alkynyl, which latter three groups are optionally substituted by one or more substituents selected from X⁴;
each R⁶ independently represents halo, —R¹¹—OR⁹, —R¹¹—CN, —R¹¹—NO₂, —R¹¹—C(O)OR⁹, —R¹¹—N(R⁹)R¹⁰, —R¹¹—C(O)N(R⁹)R¹⁰, —R¹¹—N(R^{w3})C(O)R⁹, —R¹¹—N(R^{w3})C(O)N(R⁹)R¹⁰, —R¹¹—N(R^{w3})S(O)ₜR^{9x}, —R¹¹—N(R^{w3})S(O)ₜOR^{9x}, —R¹¹—OC(O)R⁹, —R¹¹—OC(O)N(R⁹)R¹⁰, —R¹¹—OS(O)P^{9x}, —R¹¹—S(O)ₚR⁹, —R¹¹—S(O)ₜN(R^{w3})R⁹, —R¹¹—S(O)ₜOR⁹; —R¹¹—Si(R¹⁶)₃, C₁₋₁₂ alkyl, C₁₋₁₂ alkenyl, C₁₋₁₂ alkynyl, C₃₋₁₅ cycloalkyl and/or heterocyclyl which latter five groups are optionally substituted by one or more substituents selected from X⁵; or
any two R⁶ groups, or R² and any R⁶ group, may be linked together to form a further ring, which is formed either by the two relevant groups being linked together by a direct bond or C₁₋₅ alkylene;

each R⁷ independently represents halo, —R¹¹—OR⁹, —R¹¹—CN, —R¹¹—NO₂, —R¹¹—C(O)OR⁹, —R¹¹—N(R⁹)R¹⁰, —Rn—C(O)N(R⁹)R¹⁰, —R¹¹—N(R^{w3})C(O)R⁹, —R¹¹—N(R^{w3})C(O)N(R⁹)R¹⁰, —R¹¹—N(R^{w3})S(O)ₜR^{9x}, —R¹¹—N(R^{w3})S(O)ₜOR^{9x}, —R¹¹—OC(O)R⁹, —R¹¹—OC(O)N(R⁹)R¹⁰, —R¹¹—OS(O)ₜR^{9x}, —R¹¹—S(O)ₚR⁹, —R¹¹—S(O)ₜN(R^{w3})R⁹, —R¹¹—S(O)ₜOR⁹; —R¹¹—Si(R¹⁶)₃, C₁₋₁₂ alkyl, C₁₋₁₂ alkenyl, C₁₋₁₂ alkynyl, C₃₋₁₅ cycloalkyl and/or heterocyclyl which latter five groups are optionally substituted by one or more substituents selected from X⁵;

T^{z} represents a direct bond, —N(R^{w1})— or —C(O)N(R^{w2})—;
each R^{9x} independently represents, on each occasion when used herein, C₁₋₁₂ alkyl, C₂₋₁₂ alkenyl, C₂₋₁₂ alkynyl (which latter three groups are optionally substituted by one or more substituents selected from X⁶), -A⁵-O-A⁶ and/or -A⁷-B⁷;
each R⁹, R¹⁰, R^{w1}, R^{w2} and R^{w3} independently represent, on each occasion when used herein, hydrogen, C₁₋₁₂ alkyl, C₂₋₁₂ alkenyl, C₂₋₁₂ alkynyl (which latter three groups are optionally substituted by one or more substituents selected from X⁶), -A⁶-O-A⁶ and/or -A⁷-B⁷;
or R⁹ and R¹⁰, together with the carbon or nitrogen atom to which they are both attached, may be linked together to form a cycloalkyl or heterocyclyl group (both of which are optionally substituted by one or more substituents selected from Z^{1a}) or an aryl or heteroaryl group (both of which are optionally substituted by one or more substituents selected from Z^{1b}); and
each R¹¹ independently represents, on each occasion when used herein, a direct bond or R⁸;
A¹, A^{1a}, A^{1b}, A^{1c}, A^{1d}, A^{1e}, A⁴ and A⁵ independently represent C₁₋₁₂ alkylene, C₂₋₁₂ alkenylene or C₂₋₁₂ alkynylene, which latter three groups are optionally substituted by one or more substituents selected from X⁷;
A², A³ and A⁷ independently represent a direct bond, C₁₋₁₂ alkylene, C₂₋₁₂ alkenylene or C₂₋₁₂ alkynylene, which latter three groups are optionally substituted by one or more substituents selected from X⁸;
A⁶ represents C₁₋₁₂ alkyl, C₂₋₁₂ alkenyl or C₂₋₁₂ alkynyl, all of which are optionally substituted by one or more substituents selected from X⁹;
each R⁸ independently represents, on each occasion when used herein, C₁₋₁₂ alkylene, C₂₋₁₂ alkenylene or C₂₋₁₂ alkynylene, all of which are optionally substituted by one or more substituents selected from X¹⁰;
B¹ represents heteroaryl (optionally substituted by one or more substituents selected from Z^{2a}) or heterocyclyl (optionally substituted by one or more substituents selected from Z^{2b});
B², B³ and B⁷ independently represent, on each occasion when used herein, aryl (optionally substituted by one or more substituents selected from Y¹), cycloalkyl, such as C₃₋₁₅ cycloalkyl, (which cycloalkyl group is optionally substituted by one or more substituents selected from Z³), heterocyclyl (optionally substituted by one or more substituents selected from Z^{4a}) or heteroaryl (optionally substituted by one or more substituents selected from Z^{4b});
B⁴ represents aryl optionally substituted by one or more substituents selected from Y²;
X¹, X², X³, X⁴, X⁵, X⁶, X⁷, X⁸, X⁹ and X¹⁰ independently represent, on each occasion when used herein, G¹, aryl (optionally substituted by one or more T¹ substituents), C₃₋₁₅ cycloalkyl (optionally substituted by one or more T² substituents), heterocyclyl (optionally substituted by one or more T³ substituents), heteroaryl (optionally substituted by one or more T⁴ substituents), ═O, —Si(R¹⁶)₃, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶, —S(O)ₜOR¹⁶, —S(O)ₚR¹⁶, —S(O)ₜN(R¹⁴)₂, —N(R¹⁴)C(O)N(R¹⁴)₂, —N(R¹⁴)S(O)ₜOR¹⁶, —OC(O)N(R¹⁴)₂ and/or OS(O)ₜR^{9x};
Y¹ and Y² independently represent, on each occasion when used herein, -A^{x}-B^{y}, G¹, G², —R¹⁵—OR¹⁷-N(R¹⁴)₂ and/or —R¹⁵—O—R¹⁷—N(R¹⁴)S(O)ₜR¹⁶;
Z^{1a}, Z^{1b}, Z^{2a}, Z^{2b}, Z³, Z^{4a} and Z^{4b} independently represent, on each occasion when used herein, G¹, ═O, ═S, -A^{x}-B^{y} and/or G²;

G¹ represents C₁₋₁₂ alkyl (optionally substituted by one or more substituents selected from T⁵), C₂₋₁₂ alkenyl, C₂₋₁₂ alkynyl (which latter two groups are optionally substituted by one or more substituents selected from T⁶), halo, —CN, —NO₂ or ═O;

G² represents -A^{x}-B^{x}, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶, —R¹⁵—S(O)ₜOR¹⁶, —R¹⁵—S(O)ₚR¹⁶ and/or —R¹⁵—S(O)ₜN(R¹⁴)₂;
A^{x} represents, on each occasion when used herein, a direct bond or C₁₋₁₂ alkylene optionally substituted by one or more halo or ═O substituents;
B^{x} represents aryl or heteroaryl, which groups are optionally substituted by one or more substituents selected from T⁷ and T⁸, respectively;
B^{y} represents cycloalkyl or heterocyclyl, both of which are optionally substituted by one or more substituents selected from halo, C₁₋₆ alkyl, C₂₋₆ alkenyl, C₂₋₆ alkynyl (which latter three groups are optionally substituted by one or more halo substituents), —OCH₃, —OCHF₂, —OCF₃ and/or ═O;
T¹, T⁴, T⁵, T⁶, T⁷ and T⁸ independently represent halo, C₁₋₆ alkyl, C₂₋₆ alkenyl, C₂₋₆ alkynyl (which latter three groups are optionally substituted by one or more substituents selected from Q^{x1}), —OH, —O—C₁₋₆ alkyl, —OC₂₋₆ alkenyl, —OC₂₋₆ alkynyl (which latter three groups are optionally substituted by one or more substituents selected from Q^{x2}), —N(R^{w})₂, —NO₂ and/or —CN; and/or
T⁵ and T⁶ may alternatively or additionally represent ═O;
T² and T³ independently represent halo, C₁₋₆ alkyl, C₂₋₆ alkenyl, C₂₋₆ alkynyl (which latter three groups are optionally substituted by halo), —OCH₃, —OCHF₂, —OCF₃ and/or ═O;
Q^{x1} and Q^{x2} independently represent halo, —OCH₃, —OCHF₂, —OCF₃, —N(R^{w})₂ and/or ═O;
each R^{w} independently represents, on each occasion when used herein, hydrogen, C₁₋₆ alkyl, C₂₋₆ alkenyl, C₂₋₆ alkynyl, which latter three groups are optionally substituted by one or more substituents selected from halo, —OCH₃, —OCHF₂, —OCF₃ and/or ═O; or
two R^{w} groups, when attached to the same nitrogen atom, may be linked together to form, together with the nitrogen atom to which they are necessarily attached, a 5- or 6-membered ring, optionally containing a further heteroatom and optionally substituted by one or more substituents selected from fluoro, —CH₃ and ═O;
t represents, on each occasion when used herein, 1 or 2;
p represents, on each occasion when used herein, 0, 1 or 2;
each R¹⁴ independently represents, on each occasion when used herein, hydrogen, -A^{x1}-B^{x1}, C₁₋₁₂ alkyl, C₂₋₆ alkenyl or C₂₋₆ alkynyl, which latter three groups are optionally substituted by one or more substituents selected from E¹;
each R¹⁵ independently represents, on each occasion when used herein, a direct bond, C₁₋₁₂ alkylene or C₂₋₁₂ alkenylene, which latter two groups are optionally substituted by one or more substituents selected from halo, —OCH₃, —OCHF₂, —OCF₃ and ═O;
each R¹⁶ independently represents, on each occasion when used herein, C₁₋₁₂ alkyl, C₂₋₆ alkenyl, C₂₋₆ alkynyl (which latter three groups are optionally substituted by one or more halo and/or ═O groups) or -A^{y1}-B^{y1};
R¹⁷ represents, on each occasion when used herein, C₁₋₁₂ alkylene or C₂₋₁₂ alkenylene, both of which are optionally substituted by one or more substituents selected from halo and ═O;
A^{x1} and A^{y1} independently represent a direct bond or C₁₋₁₂ (e.g. C₁₋₆) alkylene optionally substituted by one or more halo and/or ═O groups;
B^{x1} and B^{y1} independently represent cycloalkyl (e.g. C₃₋₁₅ cycloalkyl), heterocyclyl (which latter two groups are optionally substituted by one or more substituents selected from halo and ═O), aryl or heteroaryl (which latter two groups are optionally substituted by one or more halo atoms);
E¹ represents halo, —CN, —NO₂, ═O, —OR¹⁸, —OC(O)—R¹⁸, —N(R¹⁸)₂, —C(O)R¹⁸, —C(O)OR¹⁸, —C(O)N(R¹⁸)₂, —N(R¹⁸)C(O)OR¹⁹, —N(R¹⁸)C(O)R¹⁹, —N(R¹⁸)S(O)ₜ₁R¹⁹, —S(O)ₜ₁OR¹⁹, —(O)ₚ₁R¹⁹, —S(O)ₜ₁N(R¹⁸)₂, —N(R¹⁸)C(O)N(R¹⁸)₂, —N(R¹⁸)S(O)ₜ₁OR^{19x}, —OC(O)N(R¹⁸)₂, —OS(O)ₜ₁R^{19x} and/or —Si(R^{19x})₃;
each R¹⁸ and R¹⁹ independently represents, on each occasion when used herein, hydrogen, C₁₋₃ alkyl, C₂₋₃ alkenyl or C₂₋₃ alkynyl, which latter three groups are optionally substituted by one or more halo atoms;
each R^{19x} independently represents, on each occasion when used herein, C₁₋₃ alkyl, C₂₋₃ alkenyl or C₂₋₃ alkynyl, which latter three groups are optionally substituted by one or more halo atoms;
t1 represents, on each occasion when used herein, 1 or 2;
p1 represents 0, 1 or 2,
or a pharmaceutically acceptable salt, solvate, prodrug or polymorph thereof,
provided that:
(A) when R¹ represents methyl substituted by X¹, R² represents hydrogen, m and n both represent O, R⁴ represents methyl:
(I) when R³ represents —OR⁴ in which R⁴ represents cyclopentyl:
(i) R⁵ represents hydrogen, then X¹ does not represent unsubstituted phenyl, 2-methylphenyl, 3-methylphenyl, 4-methylphenyl, 4-isopropylphenyl, 2-chlorophenyl, 3-chlorophenyl, 3-methoxyphenyl, 3-ethoxyphenyl, 3-propoxyphenyl, 3-butoxyphenyl, 4-butoxyphenyl, 3-pentyloxyphenyl, 3-hexyloxyphenyl, 3-heptyloxyphenyl, 3-phenoxyphenyl, 4-fluorophenyl, 3-benzyloxyphenyl, 3-trifluoromethylphenyl, 4-trifluoromethylphenyl, 4-trifluoromethoxyphenyl, 3-methoxy-4-hydroxyphenyl, 3-methoxy-4-benzyloxyphenyl, 3-(4-chlorophenoxy)phenyl, 4-phenoxyphenyl, 2-chloro-5-trifluoromethylphenyl, or benzodioxol-5-yl (especially when the compound is in the (3R,5R) orientation);
(ii) R⁵ represents —C(O)OR¹⁹, in which R¹⁹ represents tert-butyl, then X¹ does not represent 3-methoxy-4-benzyloxyphenyl;
(iii) R⁵ represents isobutyl or —C(O)R⁹, in which R⁹ represents methyl or unsubstituted phenyl, then X¹ does not represent 3-methylphenyl;
(II) when R³ represents —OR⁴ in which R⁴ represents methyl:
   (i) R⁵ represents hydrogen or benzyl, then X¹ does not represent 3-methoxy-4-benzyloxyphenyl or 3-methoxy-4-hydroxyphenyl;
   (ii) R⁵ represents —C(O)OR¹⁹, in which R¹⁹ represents tert-butyl, then X¹ does not represent 3-methoxy-4-benzyloxyphenyl;
      (III) when R³ represents —OR⁴ in which R⁴ represents isopropyl:
   (i) R⁵ represents hydrogen, then X′ does not represent unsubstituted phenyl, 4-trifluoromethylphenyl or 3-benzyloxyphenyl;
      (IV) when R³ represents —OR⁴ in which R⁴ represents ethyl:
   (i) R⁵ represents hydrogen, then X′ does not represent unsubstituted phenyl, 4-fluorophenyl or 3-benzyloxyphenyl;
      (B) when R⁴ represents methyl, R², R³ and R⁵ all represent hydrogen, n represents 0, m represents 1, and the R⁶ substituent represents methyl substituted a to the —N(R⁵)— moiety, then R¹ does not represent unsubstituted methyl;
      (C) when R² represents hydrogen, m and n both represent O, R⁴ represents methyl, then R¹ does not represent hydrogen when R³ represents —OR⁴ in which R⁴ represents cyclopentyl or methyl and R⁵ represents hydrogen, benzyl or —C(O)OR¹⁰, in which R¹⁶ represents tert-butyl;
      (D) when R² represents hydrogen, m and n both represent 0, R³ represents hydrogen, R⁵ represents methyl, then R¹ does not represent hydrogen when R⁴ represents methyl substituted by X³, in which X³—C(O)N(R¹⁴)₂ and each R¹⁴ represents isopropyl,
      which compounds are hereinafter referred to as the “compounds of the invention”.

Further compounds of the invention that may be mentioned include those as defined above, but in which:

(X^{a}) R¹ and R² do not both represent hydrogen;
(X^{b}) when R² represents hydrogen, then R¹ does not represent optionally substituted benzyl (which encompasses the situation whereby when R¹ represents hydrogen, then R² does not represent optionally substituted benzyl);
(X^{c}) when R¹ represents -A¹-T^{z}-B¹, and B¹ represents a polycyclic heteroaryl group, then the point of attachment of B¹ to the T^{Z} is via a heterocyclyl ring (when the polycyclic heteroaryl group contains an unsaturated ring) or, preferably via a heteroaromatic ring, of the polycycle.

These are herein referred to as ‘proviso (X^{a})’, ‘proviso (X^{b})’ and ‘proviso (X^{c})’.

The skilled person will appreciate that in certain embodiments of the invention, some or all of the above provisos will become redundant. For example when proviso (X^{a}) is present, then the above provisos (C) and (D) are redundant. Further, when provisos (X^{b}) and (X^{c}) are both present, then proviso (A) above is redundant. Further still, where it is stated hereinafter that “when R¹ represents C₁₋₁₂ alkyl, C₂₋₁₂ alkenyl or C₂₋₁₂ alkynyl, then such groups are optionally substituted by one or more substituents selected from halo and —OH”, when taken in conjunction with proviso (X^{c}), also renders proviso (A) above redundant.

Certain chemical groups named herein are preceded by a shorthand notation indicating the total number of carbon atoms that are to be found in the indicated chemical group. For example, C₇₋₁₂ alkyl describes an alkyl group, as defined herein, having a total of 7 to 12 carbon atoms, and C₄₋₁₂ cycloalkylalkyl describes a cycloalkylalkyl group, as defined herein, having a total of 4 to 12 carbon atoms. The total number of carbons in the shorthand notation does not include carbons that may exist in substituents of the group described.

In addition to the foregoing, as used in the specification and appended claims, unless specified to the contrary, the following terms have the meaning indicated:

“Amino” refers to the —NH₂ radical;
“Cyano” refers to the —CN radical;
“Hydroxyl” refers to the —OH radical;
“Imino” refers to the ═NH substituent;
“Nitro” refers to the —NO₂ radical;
“Oxo” refers to the ═O substituent;
“Thioxo” refers to the ═S substituent;
“Trifluoromethyl” refers to the —CF₃ radical.

Further, “alkyl” refers to cycloalkyl (where there is a minimum of three carbon atoms) or, preferably, a straight or branched hydrocarbon chain radical consisting of carbon and hydrogen atoms, containing no unsaturation. C₁₋₁₂ alkyl refers to such alkyl groups having from one to twelve carbon atoms, preferably one to eight carbon atoms and, more preferably, one to six carbon atoms, and which group is attached to the rest of the molecule by a single bond. Examples of alkyl groups include methyl, ethyl, n-propyl, 1-methylethyl (isopropyl), n-butyl, n-pentyl, 1,1-dimethylethyl (t-butyl), 3-methylhexyl, 2-methylhexyl, and the like.

The term “alkenyl” refers to cycloalkyl containing at least one double bond, or, preferably, refers to a straight or branched hydrocarbon chain radical group consisting of carbon and hydrogen atoms, containing at least one double bond. C₂₋₁₂ alkenyl refers to such alkenyl groups having from two to twelve carbon atoms, preferably one to eight carbon atoms and, more preferably, one to six carbon atoms, and which group is attached to the rest of the molecule by a single bond. Examples of alkenyl groups include ethenyl, prop-1-enyl, but-1-enyl, pent-1-enyl, penta-1,4-dienyl, and the like.

The term “alkynyl” refers to cycloalkyl containing at least one triple bond, or, preferably, refers to a straight or branched hydrocarbon chain radical group consisting of carbon and hydrogen atoms, containing at least one triple bond and optionally one or more double bonds. C₂₋₁₂ alkynyl refers to such alkynyl groups having from two to twelve carbon atoms, preferably one to eight carbon atoms and, more preferably, one to six carbon atoms, and group which is attached to the rest of the molecule by a single bond. Examples of alkynyl groups include ethynyl, prop-1-ynyl, but-1-ynyl, pent-1-ynyl, penta-1-en-4-ynyl, and the like.

The term “alkoxy” when used herein refers to a —O—C₁₋₁₂ alkyl, in which the C₁₋₁₂ alkyl group is as defined above (e.g. see the definition of C₁₋₁₂ alkyl when employed in respect of R¹). For example, the relevant C₁₋₁₂ alkyl group represents C₁₋₁₂ alkyl optionally substituted by one or more substituents selected from X¹.

The term “alkylene” or “alkylene chain” refers to cycloalkylene (when there is a minimum of three carbon atoms) or, preferably, a straight or branched divalent hydrocarbon chain linking the rest of the molecule to a radical group, consisting of carbon and hydrogen, and containing no unsaturation. C₁₋₁₂ alkylene refers to such alkylene groups having from one to twelve carbon atoms, e.g., methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon or any two carbon atoms within the chain.

The term “alkenylene” or “alkenylene chain” refers to a cycloalkylene group containing at least one double bond, or, preferably, refers to a straight or branched divalent hydrocarbon chain linking the rest of the molecule to a radical group, consisting of carbon and hydrogen, containing at least one double bond. C₂₋₁₂ alkenylene refers to such alkenylene groups having from two to twelve carbon atoms, e.g., ethenylene, propenylene, n-butenylene, and the like. The alkenylene chain is attached to the rest of the molecule through a single bond and to the radical group through a double bond or a single bond. The points of attachment of the alkenylene chain to the rest of the molecule and to the radical group can be through one carbon or any two carbons within the chain.

The term “alkynylene” or “alkynylene chain” refers to a cycloalkylene group containing at least one triple bond, or, preferably, refers to a straight or branched divalent hydrocarbon chain linking the rest of the molecule to a radical group, consisting of carbon and hydrogen, containing at least one triple bond. C₂₋₁₂ alkynylene refers to such alkynylene groups having from two to twelve carbon atoms, e.g., propynylene, n-butynylene, and the like. The alkynylene chain is attached to the rest of the molecule through a single bond and to the radical group through a double bond or a single bond. The points of attachment of the alkynylene chain to the rest of the molecule and to the radical group can be through one carbon or any two carbons within the chain.

When alkyl, alkenyl, alkynyl, alkylene, alkenylene or alkynylene groups are substituted by a cyclic group, then the point of attachment of the cyclic substituent may be via a single carbon atom.

Unless otherwise specified, alkyl, alkenyl, alkynyl, alkylene, alkenylene and alkynylene groups that are mentioned (e.g. in the definition of R¹, R², R³, R⁴, R⁵, R⁶, R⁷, R⁸, R⁹, R¹⁰ and/or R¹¹) may be optionally substituted by one or more (e.g. X⁸, X⁹ or X¹⁰ (as appropriate).

“Aryl” refers to a hydrocarbon ring system radical comprising from six to eighteen carbon atoms and at least one aromatic ring. For purposes of this invention, the aryl radical may be a monocyclic, bicyclic, tricyclic or tetracyclic ring system, which may included fused or bridged ring systems. Aryl radicals include, but are not limited to aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Particular aryl groups that may be mentioned include benzene, naphthene and the like, such as 1,2,3,4-tetrahydronaphthene, indane, indene and fluorene.

Unless otherwise specified, aryl groups that are mentioned (e.g. in the definitions of R⁴ or, preferably, R¹, R³, R⁵, R⁹ or R¹⁰) may be optionally substituted by one or more (e.g. one) Y group (i.e. Y¹ or Y²).

The term “cycloalkyl” refers to a (e.g. stable) non-aromatic monocyclic or polycyclic hydrocarbon radical consisting of carbon and hydrogen atoms, which may include fused or bridged ring systems. C₃₋₁₅ cycloalkyl refers to such cycloalkyl groups having from three to fifteen carbon atoms, preferably having from three to ten carbon atoms (i.e. C₃₋₁₀ cycloalkyl), and which group is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like. Further cycloalkyl groups that may be mentioned include C₃₋₇ (e.g. C₃₋₆) cycloalkyl groups.

When cycloalkyl, or other cyclic, groups are further substituted by a further cyclic group, then the point of attachment of the cyclic substituent may be via a single carbon atom, so forming a spiro-cyclic compound.

Unless otherwise specified, cycloalkyl groups that are mentioned (e.g. in the definitions of R¹, R³, R⁴, R⁹ or R¹⁰) may be optionally substituted by one or more (e.g. one) Z³ group.

“Halo” refers to halogen and preferably, bromo, chloro, fluoro or iodo.

When used herein, the terms C₁₋₁₂ haloalkyl, C₂₋₁₂ haloalkenyl and C₂₋₁₂ haloalkynyl refer to C₁₋₁₂ alkyl, C₂₋₁₂ alkenyl or C₂₋₁₂ alkynyl, respectively, all of which are as defined herein, but which are substituted by one or more halo groups.

Alkyl groups that may be substituted with halo atoms (i.e. “haloalkyl” groups) include, e.g., trifluoromethyl, difluoromethyl, trichloromethyl, 2,2,2-trifluoroethyl, 1-fluoromethyl-2-fluoroethyl (1,3-difluoro-2-propyl), 3-bromo-2-fluoropropyl, 1-bromomethyl-2-bromoethyl (1,3-dibromo-2-propyl), and the like. “Haloalkenyl” groups include, e.g., 2,2-difluoroethenyl, 3-chloroprop-1-enyl, and the like. “Haloalkynyl” groups include, e.g., 3-chloroprop-1-ynyl, and the like.

The term ‘hydroxyalkyl’ when used herein refers to a C₁₋₁₂ alkyl group, as defined herein, but which is substituted by one or more hydroxy (i.e. —OH) groups.

The term “heterocyclyl” refers to a (e.g. stable) 3- to 18-membered non-aromatic ring radical, which consists of two to twelve carbon atoms and from one to six heteroatoms selected from the group consisting of nitrogen, oxygen and sulfur. The total number of atoms in the ring system may be between three and twelve (e.g. between five and ten). Unless specifically stated otherwise in the specification, the heterocyclyl radical may be a monocyclic, bicyclic, tricyclic or tetracyclic (e.g. monocyclic or bicyclic) ring system, which may include fused or bridged ring systems. The nitrogen, carbon or sulfur atoms in the heterocyclyl radical may be optionally oxidised, the nitrogen atom may be optionally quaternized, and the heterocyclyl radical may be partially or fully saturated. The heterocyclyl group may therefore contain one or more double and/or triple bonds. Heterocyclyl groups that may be mentioned include, but are not limited to 7-azabicyclo-[2.2.1]heptanyl, 6-azabicyclo[3.1.1]heptanyl, 6-azabicyclo[3.2.1]-octanyl, 8-azabicyclo[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo[3.2.1]-octanyl, oxetanyl, oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thiiranyl, thiolanyl, thiomorpholinyl, trithianyl (including 1,3,5-trithianyl), tropanyl and the like. Preferred examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Optional substituents on such groups may be attached on any atom. Polycyclic heterocyclyl groups (e.g. when B¹ represents a polycyclic heterocyclyl) are preferably attached to the rest of the molecule via a heterocyclyl ring of the polycyclic ring system, i.e. a ring that contains at least one heteroatom (e.g. when the “-T^{z}-B¹” moiety is present, in which B¹ is a polycyclic heterocyclyl group, then it is attached to V).

When heterocyclyl groups are further substituted by a cyclic group, then the point of attachment of the cyclic substituent may be via a single atom, so forming a spiro-cyclic compound.

Unless otherwise specified, heterocyclyl groups that are mentioned (e.g. in the definitions of R⁴ or, preferably, R¹, R³, R⁹ or R¹⁰) may be optionally substituted by one or more (e.g. one) Z group (i.e. Z^{1a}, Z^{2b} or Z^{4a}).

The term “heteroaryl” refers to a 5- to 18-membered partially or fully aromatic ring radical (i.e. when the heteroaryl group is polycyclic, then at least one of the rings is aromatic), which consists of one to seventeen carbon atoms and from one to ten heteroatoms selected from the group consisting of nitrogen, oxygen and sulfur. Heteroaryl groups may have between five and fourteen (e.g. between five and ten) members, in which at least one (e.g. one to four) of the atoms in the ring system is/are (a) heteroatom. For purposes of this invention, the heteroaryl radical may be a monocyclic, bicyclic, tricyclic or tetracyclic (e.g. monocyclic, bicyclic or tricyclic) ring system, which may include fused or bridged ring systems. However, when the heteroaryl radical is polycyclic (i.e. bicyclic, tricyclic or tetracyclic), then the point of attachment of the heteroaryl group to the other relevant moiety of the compound of formula I is preferably via a heterocyclyl ring (i.e. a non-aromatic ring containing at least one heteroatom) or, more preferably, a heteroaromatic ring (i.e. an aromatic ring containing at least one heteroatom) of the polycycle. This situation is particularly preferred when there is a “-T^{z}-B¹” moiety present in which B¹ represents a polycyclic heteroaryl group (in which case B¹ is preferably attached to T^{z} via a heteroaromatic ring of the polycycle). One or more nitrogen, carbon or sulfur atoms (e.g. nitrogen atoms) in the heteroaryl radical may be optionally oxidized, and the nitrogen atom may be optionally quaternized (provided that, when the heteroaryl ring is polycyclic, then the point of attachment with the rest of the compound of formula I is preferably via a ring that remains heteroaromatic). Examples of such groups include, but are not limited to, 1,3-dihydroindol-2-one-yl, 2,3-dihydrobenzo[1,4]dioxinyl, benzo[1,4]oxazinyl, pyrrolopyridinyl (e.g. pyrrolo[2,3-b]pyridinyl) or, more preferably, azepinyl, acridinyl, benzimidazolyl, benzthiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrrolyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Particular examples of heteroaryl groups that may be mentioned include benzodioxanyl, benzodioxepinyl, benzodioxolyl (including 1,3-benzodioxolyl), benzofurazanyl, benzoxadiazolyl (including 2,1,3-benzoxadiazolyl), benzoxazinyl (including 3,4-dihydro-2H-1,4-benzoxazinyl), benzomorpholinyl, benzoselenadiazolyl (including 2,1,3-benzoselenadiazolyl), chromanyl, imidazopyridyl (e.g. imidazo[1,2-a]pyridyl), indolinyl, isobenzofuranyl, isochromanyl, isoindolinyl, isoindolyl, isothiochromanyl, phenazinyl, phenothiazinyl, quinolizinyl, quinoxalinyl, thiochromanyl, thiazolopyridyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), or, preferably, acridinyl, benzimidazolyl, benzofuranyl, benzothiazolyl, benzothienyl (i.e. benzothiophenyl), benzotriazolyl, benzoxazolyl, carbazolyl, cinnolinyl, furanyl, imidazolyl, indazolyl, indolyl, indolizinyl, isoquinolyl, isothiazolyl, isoxazolyl, naphthyridinyl (including 1,6-naphthyridinyl, 1,5-naphthyridinyl and 1,8-naphthyridinyl), oxadiazolyl (including 1,2,3-oxadiazolyl, 1,2,4-oxadiazolyl and, preferably, 1,3,4-oxadiazolyl), oxazolopyridinyl (e.g. oxazolo[5,4-b]pyridinyl, oxazolo[5,4-c]pyridinyl, oxazolo[4,5-b]pyridinyl, oxazolo[4,5-c]pyridinyl), oxazolyl, phthalazinyl, pteridinyl, purinyl, pyrazinyl, pyrazolyl, pyridinyl, pyridazinyl, pyrimidinyl, pyrrolyl, quinazolinyl, quinolinyl, quinoxalinyl, thiadiazolyl (including 1,2,3-thiadiazolyl, 1,2,4-thiadiazolyl and, preferably, 1,3,4-thiadiazolyl), thiazolyl, tetrazolyl, thiophenyl (i.e. thienyl), triazinyl, and triazolyl (including 1,2,3-triazolyl and 1,2,4-triazolyl).

Unless otherwise specified, heteroaryl groups that are mentioned (e.g. in the definitions of R⁴ or, preferably, R¹, R³, R⁹ or R¹⁰) may be optionally substituted by one or more (e.g. one) Z group (i.e. Z^{1b}, Z^{2a} or Z^{4b}).

As stated above m, when used in respect of the term “—(R⁶)ₘ”, may represent 0, 1, 2, 3, 4 or 5. For the avoidance of doubt, this means that the piperidin-2-one ring of the compound of formula I may contain no further R⁶ substituents (when m represent 0), or, may contain up to five R⁶ substituents at any of the carbon atoms of the piperidin-2-one ring one on which a substituent is not currently specified (i.e. on any carbon atom that is presently substituted with only a hydrogen atom). Similar logic applies to the term “—(R⁷)ₙ”, which means that there are three optional substituents present at the free positions of the relevant phenyl ring.

It is stated above that any two (of the five possible) R⁶ groups on the requisite piperidinone ring of the compound of formula I may be linked together to form a further ring, and such groups may be linked together by a direct bond or a C₁₋₅ alkylene linker group. The skilled person will appreciate that when the two relevant R⁶ groups are on the same or adjacent carbon atoms, then they cannot be linked together by a direct bond to form a further ring (rather, they may only be linked by the C₁₋₅ alkylene group). The two relevant R⁶ groups may be located on the same carbon atom of the piperidinone ring, in which case they may be linked to form a spiro-cyclic compound. The two relevant R⁶ groups may also be located on adjacent carbon atoms of the piperidinone ring, so forming a non-bridged fused bicyclic system. Alternatively, the two relevant R⁶ groups may be located on non-adjacent carbon atoms (and also not on the same carbon atom), so forming a bridged bicyclic ring structure. Similar rings may be formed between R² and adjacent or non-adjacent R⁶ groups.

For the avoidance of doubt, in cases in which the identity of two or more substituents in a compound of formula I may be the same, the actual identities of the respective substituents are not in any way interdependent. For example, in the situation in which there are two X¹ (or two Z^{1a}) substituents present, then the respective X¹ (or Z^{1a}) groups in question may be the same or different. Further, in compounds of formula I, the integer R⁴ is necessarily present, as is the integer R³, in which R³ may represent —OR⁴. In these instances, the identities of each R⁴ substituent is also not to be regarded as being interdependent. Similar logic also applies to the definitions of e.g. R⁸, R⁹, etc. Further still, when for example X¹ and X⁴ are each substituted with a G¹ group, then the identities of that G¹ group is also not to be regarded as being interdependent, i.e. the two G¹ moieties may be the same or different, and when both G¹ moieties represent C₁₋₁₂ alkyl substituted by T⁵, then the T⁵ integers in each moiety may also be the same or different.

It may be stated herein that various groups are optionally substituted. The skilled person will appreciate that substituents may only be present at a particular position if the rules of valency are adhered to. For example, where it is stated herein that a heteroaryl group may be substituted with an oxo or thioxo group, then the skilled person will appreciate that this is not possible on a carbon atom of the ring system in which the carbon atom is already attached to a double (and a single) bond.

“Prodrugs” is meant to indicate a compound that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof, but is converted in vivo to an active compound of the invention. By the term “prodrug”, we therefore include compounds that form a compound of the invention, in an experimentally-detectable amount, within a predetermined time (e.g. about 1 hour), following oral or parenteral administration. Prodrugs are typically rapidly transformed in vivo to yield the parent compound of the invention, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)). A discussion of prodrugs is provided in Higuchi, T., et al., “Pro-drugs as Novel Delivery Systems,” A.C.S. Symposium Series, Vol. 14, and in Bioreversible Carriers in Drug Design, Ed. Edward B. Roche, American Pharmaceutical Association and Pergamon Press, 1987, both of which are incorporated in full by reference herein.

The term “prodrug” is also meant to include any covalently bonded carriers, which release the active compound of the invention in vivo when such prodrug is administered to a mammalian subject. Prodrugs of a compound of the invention may be prepared by modifying functional groups present in the compound of the invention in such a way that the modifications are ‘cleaved’ (i.e. the modified functional group reverts to the original functional group) for example in vivo (i.e. it may be metabolised in the body), to the parent compound of the invention. Prodrugs include compounds of the invention wherein a hydroxy, amino or mercapto group is bonded to any group that, when the prodrug of the compound of the invention is administered to a mammalian subject, cleaves to form a free hydroxy, free amino or free mercapto group, respectively. Examples of prodrugs include, but are not limited to, acetate, formate and benzoate derivatives of alcohol or amide derivatives of amine functional groups in the compounds of the invention and the like.

The invention disclosed herein is also meant to encompass all pharmaceutically acceptable compounds of the invention being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I and ¹²⁵I, respectively. These radiolabelled compounds could be useful to help determine or measure the effectiveness of the compounds. Certain isotopically-labelled compounds of the invention, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁸O and ¹³N, can be useful in Positron Emission Topography (PET) studies. Isotopically-labelled compounds of the invention can generally be prepared by conventional techniques known to those skilled in the art or by processes analogous to those described in the Examples and Preparations as set out below using an appropriate isotopically-labelled reagent in place of the non-labelled reagent previously employed.

The invention disclosed herein is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the invention includes compounds produced by a process comprising contacting a compound of this invention with a mammal for a period of time sufficient to yield a metabolic product thereof. Such products are typically identified by administering a radiolabelled compound of the invention in a detectable dose to an animal, such as rat, mouse, guinea pig, monkey, or to human, allowing sufficient time for metabolism to occur, and isolating its conversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

“Mammal” includes humans and both domestic animals such as laboratory animals and household pets, (e.g. cats, dogs, swine, cattle, sheep, goats, horses, rabbits), and non-domestic animals such as wildlife and the like.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier, for example one which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, 2-dimethylaminoethanol (deanol), 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallisations produce a solvate of the compound of the invention. As used herein, the term “solvate” refers to an aggregate that comprises one or more molecules of a compound of the invention with one or more molecules of solvent. The solvent may be water, in which case the solvate may be a hydrate. Alternatively, the solvent may be an organic solvent. Thus, the compounds of the present invention may exist as a hydrate, including a monohydrate, dihydrate, hemihydrate, sesquihydrate, trihydrate, tetrahydrate and the like, as well as the corresponding solvated forms. The compound of the invention may be true solvates, while in other cases, the compound of the invention may merely retain adventitious water or be a mixture of water plus some adventitious solvent.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

“Therapeutically effective amount” refers to that amount of a compound of the invention which, when administered to a mammal, preferably a human, is sufficient to effect treatment, as defined below, of a disease or condition of interest in the mammal, preferably a human. The effect may be objective (i.e. measurable by some test or marker) or subjective (i.e. the subject gives an indication of or feels an effect).

The amount of a compound of the invention which constitutes a “therapeutically effective amount” will vary depending on several factors including the compound, the condition and its severity, the manner of administration, and the type of mammal to be treated (e.g. the amount may vary depending on the species, age, weight, sex, renal function, hepatic function and response of the mammal), but can be determined routinely by one of ordinary skill in the art having regard to his own knowledge and to this disclosure.

“Treating” or “treatment” as used herein refers to the therapeutic treatment and/or prophylactic treatment of the disease or condition of interest in a mammal, preferably a human, having the disease or condition of interest. Such terms therefore include:

(i) preventing the disease or condition from occurring in a mammal, in particular, when such mammal is predisposed to the condition but has not yet been diagnosed as having it (e.g. prophylactic treatment); or
(ii) therapeutic treatment, i.e. treatment of the disease itself, (e.g. complete or partial treatment), which includes:
(a) inhibiting the disease or condition, i.e., arresting its development;
(b) relieving the disease or condition, i.e., causing regression of the disease or condition; or
(c) relieving the symptoms resulting from the disease or condition, e.g., relieving swelling without addressing the underlying disease or condition.

As used herein, the terms “disease” and “condition” may be used interchangeably or may be different in that the particular malady or condition may not have a known causative agent (so that etiology has not yet been worked out) and it is therefore not yet recognised as a disease but only as an undesirable condition or syndrome, wherein a more-or-less specific set of symptoms have been identified by clinicians.

As used herein, the following abbreviations have the indicated meanings:

### Abbreviation Full Name

ANOVA Analysis of Variance
Boc t-butoxycarbonyl
BOP benzotriazol-1-yl-oxy-tris-(dimethylamino)phosphonium hexa-fluorophosphate
cAMP Cyclic adenosine 3′-5′-monophosphate
CD Cluster designation
cGMP Cyclic guanosine 3′-5′-monophosphate
CIA Collagen Induced Arthritis
CNS Central Nervous System
COX Cyclooxygenase
CRE cAMP response element
DMAP 4-Dimethylaminopyridine
DMARD Disease modifying anti-rheumatic drug
DMF N,N-Dimethylformamide
DMPU N,N-dimethyl propylene urea
DMSO dimethylsulfoxide
DNA Deoxyriboneucleic acid
EC50 Concentration at which a 50% of maximum observable effect is noted
EDTA Ethylenediaminotetraacetic acid
ELISA Enzyme-linked immunosorbent assay
EtOAc Ethyl acetate
EtOH Ethyl alcohol
FBS Fetal bovine serum
FCS Fetal calf serum
H & E Haematoxylin and eosin
HARBS High affinity rolipram binding site
HPLC High pressure liquid chromatography
i.p. intraperitoneal
IBD Inflammatory bowel disease
IBMX 3-isobutyl-1-methylxanthine
IC Inhibitory concentration
IC₅₀ Concentration at which 50% inhibition is observed
IFN-γ Interferon gamma
IL Interleukin LAH Lithium aluminum hydride
LDA Lithium diisopropylamide
LPS lipopolysaccharide
LTB₄ Leukotriene B₄.
luc luciferase
Me Methyl
MeOH Methyl alcohol
MHC Major histocompatibility class
MLR Mixed lymphocyte reaction
MPO myeloperoxidase
Ms Methanesulfonyl
MsCl Methanesulfonyl chloride
NBS N-Bromosuccinimide
n-BuLi n-Butyllithium
n-BuSH n-Butanethiol
NF-κB Nuclear factor kappa B
NSAID Non-steroidal anti-inflammatory drug
PBS Phosphate buffered saline
PDE Phosphodiesterase
PMS Phenazine methosulfate
PMSF Phenyl methyl sulfonyl fluoride
pTsOH p-Toluenesulfonic acid monohydrate
Py Pyridine
RA Rheumatoid arthritis
RF Rheumatoid factor
Rf Retardation factor
ROS Reactive oxygen species
RPMI Rosewell Park Memorial Institute
SAR Structure activity relationship
TBAF Tetrabutylammonium fluoride
TBDMS tert-Butyldimethylsilyl
TBDMSCI tert-Butyldimethylsilyl chloride
TCR T-cell receptor
TEA Triethylamine
Tf Trifluoromethanesulfonyl
TFA Trifluoroacetic acid
THF Tetrahydrofuran
TNBS Trinitrobenzene sulfonic acid
TNF-α Tumour necrosis factor alpha
TsOH p-Toluenesulfonic acid monohydrate
μL Micro litre
μM Micro molar

As stated above, compounds of the invention may exist as a stereoisomers, enantiomers, tautomers, or mixtures thereof.

The compounds of the invention, or their pharmaceutically acceptable salts may contain one or more asymmetric centres and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D) and (L)-isomers may be prepared using chiral synthons or chiral reagents, or resolved using conventional techniques, for example, chromatography and fractional crystallisation. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers. Likewise, all tautomeric forms are also intended to be included. In certain instances, the spatial orientation of the substituents of the compounds of the invention are designated herein as α (alpha) or β (beta). For purposes of this disclosure, substituents with the α orientation are considered to be below the plane of the paper and substituents with the β orientation are considered to be above the plane of the paper.

A “stereoisomer” refers to a compound made up of, the same atoms bonded by the same bonds but having different three-dimensional structures, which are not interchangeable. The present invention contemplates various stereoisomers and mixtures thereof and includes “enantiomers”, which refers to two stereoisomers whose molecules are non-superimposeable mirror images of one another.

A “tautomer” refers to a proton shift from one atom of a molecule to another atom of the same molecule. The present invention includes tautomers of any said compounds.

Also within the scope of the invention is the use of intermediate compounds of the invention (for instance the use of such compounds in a process for preparing compounds of the invention) and the use of all polymorphs of the aforementioned species and crystal habits thereof.

Compounds of the invention include those in which:

R⁵ represents hydrogen, -A⁴-B⁴, —C(O)R⁹, —C(O)OR¹⁰ or C₁₋₁₂ alkyl optionally substituted by one or more substituents selected from X⁴;
each R⁶ independently represents halo, —R¹¹—OR⁹, —R¹¹—CN, —R¹¹—NO₂, —R¹¹—C(O)OR⁹, —R¹¹—N(R⁹)R¹⁰, —R¹¹—C(O)N(R⁹)R¹⁹ and/or C₁₋₁₂ alkyl optionally substituted by one or more substituents selected from X⁵; or
any two R⁶ groups, or R² and any R⁶ group, may be linked together to form a further ring, which is formed either by the two relevant groups being linked together by a direct bond or C₁₋₅ alkylene;
each R⁷ independently represents halo, —R¹¹—OR⁹, —R¹¹—CN, —R¹¹—NO₂, —R¹¹—C(O)OR⁹, —R¹¹—N(R⁹)R¹⁰, —R¹¹—C(O)N(R⁹)R¹⁰ and/or C₁₋₁₂ alkyl optionally substituted by one or more substituents selected from X⁵;
A⁶ represents C₁₋₁₂ alkyl optionally substituted by one or more substituents selected from X⁹;
R⁹ and R¹⁰ may only be linked together when they are both attached to a nitrogen atom, in which case, they may together form a heterocyclyl group (optionally substituted by one or more substituents selected from Z^{1a}) or a heteroaryl group (optionally substituted by one or more substituents selected from Z^{1b});
X¹, X², X³, X⁴, X⁵, X⁶, X⁷, X⁸, X⁹ and X¹⁰ independently represent, on each occasion when used herein, G¹, aryl (optionally substituted by one or more T¹ substituents), C₃₋₁₅ cycloalkyl (optionally substituted by one or more T² substituents), heterocyclyl (optionally substituted by one or more T³ substituents), heteroaryl (optionally substituted by one or more T⁴ substituents), ═O, —Si(CH₃)₃, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶, —S(O)ₜOR¹⁶, —S(O)ₚR¹⁶ and/or —S(O)ₜN(R¹⁴)₂;
G¹ represents C₁₋₁₂ alkyl (optionally substituted by one or more substituents selected from T⁵), C₂₋₁₂ alkenyl (optionally substituted by one or more substituents selected from r), halo, —CN, —NO₂ or ═O;
B^{y} represents cycloalkyl or heterocyclyl, both of which are optionally substituted by one or more substituents selected from halo, C₁₋₆ alkyl (optionally substituted by one or more halo substituents), —OCH₃, —OCHF₂, —OCF₃ and/or ═O;
T¹, T⁴, T⁵, T⁶, T⁷ and T⁸ independently represent halo, C₁₋₆ alkyl (optionally substituted by one or more substituents selected from Q^{x1}), —OH, —O—C₁₋₆ alkyl (optionally substituted by one or more substituents selected from Q^{x2}), —N(R^{w})₂, —NO₂ and/or —CN; and/or
T⁵ and T⁶ may alternatively or additionally represent ═O;
T² and T³ independently represent halo, C₁₋₆ alkyl (optionally substituted by halo), —OCH₃, —OCHF₂, —OCF₃ and/or ═O;
R^{w} resents, on each occasion when used herein, hydrogen or C₁₋₆ alkyl optionally substituted by one or more substituents selected from halo, —OCH₃, —OCHF₂, —OCF₃ and/or ═O; or
two R^{w} groups, when attached to the same nitrogen atom, may be linked together to form, together with the nitrogen atom to which they are necessarily attached, a 5- or 6-membered ring, optionally containing a further heteroatom and optionally substituted by one or more substituents selected from fluoro, —CH₃ and ═O;
each R¹⁴ independently represents, on each occasion when used herein, hydrogen, -A^{x1}-B^{x1} or C₁₋₁₂ alkyl optionally substituted by one or more substituents selected from E¹;
each R¹⁶ independently represents, on each occasion when used herein, C₁₋₁₂ alkyl (optionally substituted by one or more halo and/or ═O groups) or -A^{y1}-B^{y1};
E¹ represents halo, ═O, —OR¹⁸, —OC(O)—R¹⁸, —N(R¹⁸)₂, —C(O)R¹⁸, —C(O)OR¹⁸, —C(O)N(R¹⁸)₂, —N(R¹⁸)C(O)OR¹⁹, —N(R¹⁸)C(O)R¹⁹, —N(R¹⁸)S(O)ₜ, R¹⁹, —S(O)ₜ₁OR¹⁹, —S(O)ₚ₁R¹⁹ and/or —S(O)ₜ₁N(R¹⁸)₂;
R¹⁸ and R¹⁹ independently represent, on each occasion when used herein, hydrogen or C₁₋₃ alkyl optionally substituted by one or more halo atoms;
when E¹ represents —N(R¹⁸)S(O)ₜ₁R¹⁹, then R¹⁹ preferably represents C₂₋₃ alkenyl, C₂₋₃ alkynyl or, preferably, C₁₋₃ alkyl (all three of which are optionally substituted by one or more halo atoms);
   Y¹ and Y² independently represent, on each occasion when used herein, G¹, G², —R¹⁵—OR¹⁷—N(R¹⁴)₂ and/or —R¹⁵—O—R¹⁷—N(R¹⁴)S(O)ₜR¹⁶.

Further compounds of the invention include those in which:
R¹ represents -A¹-T^{Z}-B¹, -A^{1a}-N(R⁹)R¹⁰, -A^{1b}-OR⁹, -A^{1c}-C(O)R⁹, -A^{1d}-C(O)R⁹, or -A^{1e}-C(O)N(R⁹)R¹⁰;
   R⁵ represents hydrogen, —C(O)R⁹ or —C(O)OR¹⁰;
   when R⁵ represents C₁₋₁₂ alkyl optionally substituted by one or more substituents selected from X⁴, then X⁴ does not represent aryl;
   there is no R⁶ substituent at the 4-position of the piperidin-2-one (i.e. when m is other than 0).

Compounds of the invention that may be mentioned include those in which:

R¹ represents hydrogen, C₁₋₁₂ alkyl, C₂₋₁₂ alkenyl, C₂₋₁₂ alkynyl, C₁₋₁₂ hydroxyalkyl, C₁₋₁₂ haloalkyl, C₂₋₁₂ haloalkenyl, C₂₋₁₂ haloalkynyl, -A^{1a}-N(R⁹)R¹⁰, -A^{1b}-OR⁹, -A^{1c}-C(O)R⁹, -A^{1d}-C(O)OR⁹, -A^{1e}-C(O)N(R⁹)R¹⁰ or optionally substituted -A¹-B¹;
R² represents hydrogen, C₁₋₁₂ alkyl, C₁₋₁₂ hydroxyalkyl, C₂₋₁₂ alkenyl or C₁₋₁₂ haloalkyl; or
R¹ and R² together form ═C(R⁹)R¹⁶;
R³ represents hydrogen, —OR⁴, C₁₋₁₂ alkyl, C₂₋₁₂ alkenyl, C₂₋₁₂ alkynyl or optionally substituted -A²-B²;
each R⁴ is independently selected from the group consisting of hydrogen, —R⁸—OR⁹, —R⁸—C(O)OR⁹, C₁₋₁₂ alkyl, C₂₋₁₂ alkenyl, C₂₋₁₂ alkynyl and optionally substituted -A³-B³;
R⁵ represents hydrogen, C₁₋₁₂ alkyl, C₁₋₁₂ haloalkyl, -A⁴-B⁴, —C(O)R⁹ or —C(O)OR¹⁰;
each R⁶ and R⁷ is independently selected from the group consisting of C₁₋₁₂ alkyl, halo, C₁₋₁₂ haloalkyl, —R¹¹—OR⁹, C(O)OR⁹, —R¹¹—N(R⁹)R¹⁰ and R¹¹—C(O)N(R⁹)R¹⁰;
any two R⁶ groups, or R² and any R⁶ group, are preferably not linked together;
T^{z} represents a direct bond;
each R⁹ and R¹⁰ is independently selected from the group consisting of hydrogen, C₁₋₁₂ alkyl, C₂₋₁₂ alkenyl, C₂₋₁₂ alkynyl, C₁₋₁₂ haloalkyl, C₁₋₁₂ haloalkenyl, -A⁵-O-A⁶ (e.g. —C₁₋₁₂ alkylene-O—C₁₋₁₂ alkyl) and optionally substituted -A⁷-B⁷; or
R⁹ and R¹⁰, together with the nitrogen atom to which they are both attached, may form an optionally substituted heterocyclyl or an optionally substituted heteroaryl group;
each R⁸ independently represents straight or branched optionally substituted C₁₋₁₂ alkylene; straight or branched optionally substituted C₂₋₁₂ alkenylene; or straight or branched optionally substituted C₂₋₁₂ alkynylene;
A¹ represents C₁₋₁₂ alkylene, C₂₋₁₂ alkenylene or C₂₋₁₂ alkynylene;
B¹ represents heteroaryl or heterocyclyl;
A² represents a direct bond or C₁₋₁₂ alkylene;
B² and B⁷ independently represent aryl, cycloalkyl, heterocyclyl or heteroaryl;
A³ represents a direct bond, C₁₋₁₂ alkylene, C₂₋₁₂ alkenylene or C₂₋₁₂ alkynylene;
B³ represents cycloalkyl (e.g. C₃₋₁₅ cycloalkyl);
A⁴ represents C₁₋₁₂ alkylene;
B⁴ represents aryl;
A⁵ represents C₁₋₁₂ alkylene;
A⁶ represents C₁₋₁₂ alkyl;
A⁷ represents a direct bond or C₁₋₁₂ alkylene;
X¹ to X¹⁰ independently represent C₁₋₁₂ alkyl, C₂₋₁₂ alkenyl, halo, C₂₋₁₂ haloalkenyl, —CN, —NO₂, aryl, C₃₋₁₅ cycloalkyl, heterocyclyl, heteroaryl, ═O, —Si(CH₃)₃, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶, —S(O)ₜOR¹⁶, —S(O)ₚR¹⁶ and/or —S(O)ₜN(R¹⁴)₂;
Z^{1b}, Z^{2a} and Z^{4b} independently represent C₁₋₁₂ alkyl, C₂₋₁₂ alkenyl, C₁₋₁₂ alkoxy, halo, C₁₋₁₂ haloalkyl, C₂₋₁₂ haloalkenyl, —CN, ═O, ═S, —NO₂, -A^{x}-B^{x}, -A^{x}-B^{y}, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶, —R¹⁵—S(O)ₜOR¹⁶, —R¹⁵—S(O)ₚR¹⁶ and/or —R¹⁵—S(O)ₜN(R¹⁴)₂;
Z^{1a}, Z^{2b} and Z^{4a} independently represent C₁₋₁₂ alkyl, C₂₋₁₂ alkenyl, halo, C₁₋₁₂ haloalkyl, C₂₋₁₂ haloalkenyl, —CN, ═O, ═S, —NO₂, -A^{x}-B^{y}, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶, —R¹⁵—S(O)ₜOR¹⁶, —R¹⁵—S(O)ₚR¹⁶ and/or —R¹⁵—S(O)ₜN(R¹⁴)₂;
Z³ represents C₁₋₁₂ alkyl, C₂₋₁₂ alkenyl, halo, C₁₋₁₂ haloalkyl, C₂₋₁₂ haloalkenyl, —CN, ═O, —NO₂, -A^{x}-B^{x}, -A^{x}-B^{y}, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶, —R¹⁵—S(O)ₜOR¹⁶, —R¹⁵—S(O)ₚR¹⁶ and/or —R¹⁵—S(O)ₜN(R¹⁴)₂;
Y¹ and Y² independently represent C₁₋₁₂ alkyl, C₂₋₁₂ alkenyl, halo, C₁₋₁₂ haloalkyl, C₂₋₁₂ haloalkenyl, —CN, —NO₂, —R¹⁵—OR¹⁴, —R¹⁵—OC(O)—R¹⁴, —R¹⁵—N(R¹⁴)₂, —R¹⁵—O—R¹⁷—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶, —R¹⁵—S(O)ₜOR¹⁶, —R¹⁵—S(O)ₚR¹⁶ and/or —R¹⁵—S(O)₁N(R¹⁴)₂;
t represents, on each occasion when used herein, 1 or 2;
p represents, on each occasion when used herein, 0, 1 or 2;
A^{x} represent a direct bond or C₁₋₁₂ alkylene;
B^{x} represents aryl or heteroaryl;
B^{y} represents cycloalkyl or heterocyclyl;
when Z^{1a}, Z^{1b}, Z^{2a}, Z^{2b}, Z^{4a} or Z^{4b} represents a group containing R¹⁴, then each R¹⁴ independently represents, on each occasion when used herein, hydrogen, C₁₋₁₂ alkyl, C₂₋₁₂ alkenyl, C₁₋₁₂ haloalkyl or -A^{x1}-B^{x1};
when an X group (i.e. X¹, X², X³, X⁴, X⁵, X⁶, X⁷, X⁸, X⁹ or X¹⁰), Z³, Y¹ or Y² represents a group containing R¹⁴, then each R¹⁴ independently represents, on each occasion when used herein, hydrogen, C₁₋₁₂ alkyl, C₁₋₁₂ haloalkyl or -A^{x1}-B^{x1}; when an X group, Z³, Y¹ or Y² represents a group containing R¹⁶, then each R¹⁶ independently represents, on each occasion when used herein, C₁₋₁₂ alkyl, C₁₋₁₂ haloalkyl or -A^{y1}-B^{y1};
each R¹⁵ independently represents, on each occasion when used herein, a direct bond, C₁₋₁₂ alkylene or C₂₋₁₂ alkenylene;
when Z^{1a}, Z^{1b}, Z^{2a}, Z^{2b}, Z^{4a}, Z^{4b} represents a group containing R¹⁶, then each R¹⁶ independently represents, on each occasion when used herein, C₁₋₁₂ alkyl, C₂₋₁₂ alkenyl, C₁₋₁₂ haloalkyl or -A^{y1}-B^{y1};
R¹⁷ represents C₁₋₁₂ alkylene or C₂₋₁₂ alkenylene; and/or
A^{x1} and A^{y1} independently represent a direct bond or C₁₋₁₂ (e.g. C₁₋₆) alkylene;
B^{x1} and B^{y1} independently represent C₃₋₁₅ cycloalkyl, heterocyclyl, aryl or heteroaryl.

Compounds of the invention that may be mentioned include those in which:

R¹ represents:
hydrogen;
optionally substituted C₁₋₁₂ alkyl, C₂₋₁₂ alkenyl or C₂₋₁₂ alkynyl;

### -A^{1a}-N(R⁹)R¹⁰;

### -A^{1c}-C(O)R⁹;

### -A^{1d}-C(O)OR⁹ or -A^{1e}-C(O)N(R⁹)R¹⁰;

-A¹-T^{z}-B¹ (in which, preferably, A¹ represents C₁₋₁₂ alkylene as defined herein; T^{z} represents a direct bond; and/or B¹ represents heteroaryl as defined herein);
-A¹-T^{z}-B¹ (in which, preferably, A¹ represents C₁₋₁₂ alkylene as defined herein; T^{z} represents a direct bond; and/or B¹ represents heterocyclyl as defined herein); and/or)
R¹ and R² together represent ═C(R⁹)R¹⁰.

Particularly preferred compounds of the invention that may be mentioned include those in which R¹ represents -A¹-T^{z}-B¹ (in which, preferably, A¹ represents C₁₋₁₂ alkylene as defined herein; T^{z} represents a direct bond; and/or B¹ represents heteroaryl as defined herein).

Compounds of the invention that may be mentioned include those in which: when R² or, preferably, R¹ represents C₁₋₁₂ alkyl, O₂₋₁₂ alkenyl or C₂₋₁₂ alkynyl (and particularly C₁₋₁₂ alkyl), all of which are optionally substituted by one or more X¹ groups, then X¹ represents G¹, C₃₋₁₅ cycloalkyl (optionally substituted by one or more T² substituents), heterocyclyl (optionally substituted by one or more T³ substituents), heteroaryl (optionally substituted by one or more T⁴ substituents), ═O, —Si(R¹⁶)₃, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶, —S(O)ₜOR¹⁶, —S(O)ₚR¹⁶, —S(O)ₜN(R¹⁴)₂, —N(R¹⁴)C(O)N(R¹⁴)₂, —N(R¹⁴)S(O)ₜOR¹⁶, —OC(O)N(R¹⁴)₂ and/or —OS(O)ₜR^{9x};

when R² or, preferably, R¹ represents C₁₋₁₂ alkyl, C₂₋₁₂ alkenyl or C₂₋₁₂ alkynyl, then such groups are optionally substituted by one or more substituents selected from halo and —OH;
when R² or, preferably, R¹ represents C₁₋₁₂ alkyl, C₂₋₁₂ alkenyl or C₂₋₁₂ alkynyl, then such groups are preferably unsubstituted;
R¹ does not represent hydrogen;
R¹ represents hydrogen or, more preferably, C₁₋₁₂ alkyl, C₂₋₁₂ alkenyl, C₂₋₁₂ alkynyl, C₁₋₁₂ hydroxyalkyl, C₁₋₁₂ haloalkyl, C₂₋₁₂ haloalkenyl, C₂₋₁₂ haloalkynyl, -A^{1a}-N(R⁹)R¹⁹, -A^{1b}-OR⁹, -A^{1c}-C(O)R⁹, -A^{1d}-C(O)OR⁹, -A^{1e}-C(O)N(R⁹)R¹⁰ or optionally substituted -A¹-B¹;
X¹ represents G¹, C₃₋₁₅ cycloalkyl (optionally substituted by one or more T² substituents), heterocyclyl (optionally substituted by one or more T³ substituents), heteroaryl (optionally substituted by one or more T⁴ substituents), ═O, —Si(R¹⁶)₃, —OR¹⁴, —OC(O)—R¹⁴, —N(R¹⁴)₂, —C(O)R′⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶, —S(O)ₜOR¹⁶, —S(O)ₚR¹⁶, —S(O)ₜN(R¹⁴)₂, —N(R¹⁴)C(O)N(R¹⁴)₂, —N(R¹⁴)S(O)ₜOR¹⁶, —OC(O)N(R¹⁴)₂ and/or —OS(O)ₜR^{9x};
when R¹ and R² both represent hydrogen, then R⁵ represents -A⁴-B⁴, —C(O)R⁹, —C(O)OR¹⁰, C₁₋₁₂ alkyl, C₂₋₁₂ alkenyl or C₂₋₁₂ alkynyl, which latter three groups are e.g. optionally substituted by one or more substituents selected from X⁴.

Further compounds of the invention that may be mentioned include those in which:

when R² or, preferably, R¹ represents C₁₋₁₂ alkyl, C₂₋₁₂ alkenyl or C₂₋₁₂ alkynyl (and particularly C₁₋₁₂ alkyl), all of which are optionally substituted by one or more X¹ groups, then X¹ represents G¹, C₃₋₁₅ cycloalkyl (optionally substituted by one or more T² substituents), heterocyclyl (optionally substituted by one or more T³ substituents), heteroaryl (optionally substituted by one or more T⁴ substituents), ═O, —Si(CH₃)₃, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶, —S(O)ₜOR¹⁶, —S(O)ₚR¹⁶ and/or —S(O)ₜN(R¹⁴)₂;
X¹ represents G¹, C₃₋₁₅ cycloalkyl (optionally substituted by one or more T² substituents), heterocyclyl (optionally substituted by one or more T³ substituents), heteroaryl (optionally substituted by one or more T⁴ substituents), ═O, —Si(CH₃)₃, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶, —S(O)ₜOR¹⁶, —S(O)ₚR¹⁶ and/or —S(O)ₜN(R¹⁴)₂;
when R¹ and R² both represent hydrogen, then R⁵ represents C₁₋₁₂ alkyl (e.g. optionally substituted by one or more substituents selected from X¹), C₁₋₁₂ haloalkyl, -A⁴-B⁴, —C(O)R⁹ or —C(O)OR¹⁰.

Preferred compounds of the invention, for example, when R¹ represents hydrogen, include those in which:

m represents 0, 1 or 2;
n represents 0, 1 or 2;
R¹ represents hydrogen;
R² represents —OR⁴ or, preferably, hydrogen, C₁₋₁₂ alkyl, C₂₋₁₂ alkenyl or C₁₋₁₂ haloalkyl;
R³ represents —OR⁴ or optionally substituted -A²-B²;
A² represents a direct bond;
B² represents aryl;
each R⁴ is independently selected from the group consisting of hydrogen, —R⁸—OR⁹, —R⁸—C(O)OR⁹, C₁₋₁₂ alkyl, and optionally substituted -A³-B³;
A³ represents a direct bond;
B³ represents cycloalkyl;
R⁵ represents hydrogen, -A⁴-B⁴ or —C(O)R⁹;
A⁴ represents C₁₋₁₂ alkylene;
B⁴ represents aryl;
each R⁶ and R⁷ is independently selected from the group consisting of C₁₋₁₂ alkyl, halo, C₁₋₁₂ haloalkyl, —R¹¹—OR⁹, —R¹¹—CN, —R¹¹—NO₂, —R¹¹—C(O)OR⁹, —R¹¹—N(R⁹)R¹⁰ and —R¹¹—C(O)N(R⁹)R¹⁰;
each R⁹ and R¹⁰ is independently selected from the group consisting of hydrogen, C₁₋₁₂ alkyl, C₁₋₁₂ haloalkyl, CO₁₋₁₂ haloalkenyl, -A⁵-O-A⁶ (e.g. —C₁₋₁₂ alkylene-O—C₁₋₁₂ alkyl) and optionally substituted -A⁷-B⁷; or
R⁹ and R¹⁰, together with the nitrogen atom to which they are both attached, may form an optionally substituted heterocyclyl or an optionally substituted heteroaryl group;
A⁷ represents a direct bond or C₁₋₁₂ alkyl;
B⁷ represents cycloalkyl, aryl, heterocyclyl or heteroaryl;
each R¹¹ independently represents a direct bond or R⁸; and/or
each R⁸ independently represents straight or branched optionally substituted C₁₋₁₂ alkylene.

Further preferred compounds of the invention, for example, when R¹ represents optionally substituted C₁₋₁₂ alkyl, C₂₋₁₂ alkenyl or C₂₋₁₂ alkynyl, include those in which:

m represents 0, 1 or 2;
n represents 0, 1 or 2;
R¹ represents C₁₋₁₂ alkyl, C₂₋₁₂ alkenyl or C₂₋₁₂ alkynyl;
R² represents —OR⁴ or, preferably, hydrogen, C₁₋₁₂ alkyl, C₂₋₁₂ alkenyl or C₁₋₁₂ haloalkyl;
R³ represents hydrogen or —OR⁴;
each R⁴ is independently selected from the group consisting of hydrogen, —R⁸—OR⁹, —R⁸—C(O)OR⁹, C₁₋₁₂ alkyl, and optionally substituted -A³-B³;
A³ represents a direct bond;
B³ represents cycloalkyl;
R⁵ represents hydrogen or —C(O)R⁹;
each R⁶ and R⁷ is independently selected from the group consisting of C₁₋₁₂ alkyl, halo, C₁₋₁₂ haloalkyl, —R¹¹—OR⁹, —R¹¹—CN, —R¹¹—NO₂, —R¹¹—(C(O)OR⁹, —R¹¹—N(R⁹)R¹⁰ and —R¹¹—C(O)N(R⁹)R¹⁰;
each R⁹ and R¹⁰ is independently selected from the group consisting of hydrogen, C₁₋₁₂ alkyl, C₁₋₁₂ haloalkyl, -A⁵-O-A⁶ (e.g. —C₁₋₁₂ alkylene-O—C₁₋₁₂ alkyl) and optionally substituted -A⁷-B⁷;
A⁷ represents a direct bond or C₁₋₁₂ alkyl;
B⁷ represents cycloalkyl, aryl, heterocyclyl or heteroaryl;
each R¹¹ independently represents a direct bond or R⁸; and/or
each R⁸ independently represents straight or branched optionally substituted C₁₋₁₂ alkylene.

Further preferred compounds of the invention, for example, when R¹ represents -A^{1a}-N(R⁹)R¹⁰, -A^{1c}-C(O)R⁹, -A^{1d}-C(O)OR⁹, -A^{1e}-C(O)N(R⁹)R¹⁰ or -A¹-T^{z}-B¹, or, R¹ and R² together represent ═C(R⁹)R¹⁰, include those in which:

m represents 0, 1 or 2;
n represents 0, 1 or 2;
R¹ represents -Ala-N(R⁹)R¹⁰, -A^{1d}-C(O)OR⁹, -A^{1e}-C(O)N(R⁹)R¹⁰ or -A¹-T^{z}-B¹;
R² represents —OR⁴ or, preferably, hydrogen, C₁₋₁₂ alkyl, C₂₋₁₂ alkenyl or C₁₋₁₂ haloalkyl; or
R¹ and R² together represent ═C(R⁹)R¹⁰;
A¹, A^{1a}, A^{1c}, A^{1d} and A^{1e} independently represent straight or branched optionally substituted C₁₋₁₂ alkylene;
T^{z} represents a direct bond;
B¹ represents optionally substituted heteroaryl or optionally substituted heterocyclyl;
R³ represents hydrogen or —OR⁴;
each R⁴ is independently selected from the group consisting of hydrogen, —R⁸—OR⁹, —R⁸—C(O)OR⁹, C₁₋₁₂ alkyl, and optionally substituted -A³-B³;
A³ represents a direct bond;
B³ represents cycloalkyl;
R⁵ represents hydrogen or —C(O)R⁹;
each R⁶ and R⁷ is independently selected from the group consisting of C₁₋₁₂ alkyl, halo, C₁₋₁₂ haloalkyl, —R¹¹—OR⁹, N —R¹¹—NO₂, —R¹¹—C(O)OR⁹, —R¹¹-N(R⁹)R¹⁰ and —R¹¹—C(O)N(R⁹)R¹⁰;
each R⁹ and R¹⁰ is independently selected from the group consisting of hydrogen, C₁₋₁₂ alkyl, C₁₋₁₂ haloalkyl, C₂₋₁₂ haloalkenyl, -A⁵-O-A⁶ (e.g. —C₁₋₁₂ alkylene-O—C₁₋₁₂ alkyl) and optionally substituted -A⁷-B⁷;
A⁷ represents a direct bond or C₁₋₁₂ alkyl;
B⁷ represents cycloalkyl, aryl, heterocyclyl or heteroaryl;
each R¹¹ independently represents a direct bond or R⁸; and/or
each R⁸ independently represents straight or branched optionally substituted C₁₋₁₂ alkylene.

Further preferred compounds of the invention, for example, when R¹ represents -A^{1b}-OR⁹ in which R⁹ represents hydrogen or, preferably, R¹ represents C₁₋₁₂ hydroxyalkyl (i.e. C₁₋₁₂ alkyl substituted by one or more —OH groups), include those in which:

m represents 0, 1 or 2;
n represents 0, 1 or 2;
R¹ represents -A^{1b}-OH or, preferably, C₁₋₁₂ alkyl substituted by one or more —OH groups (i.e. C₁₋₁₂ hydroxyalkyl);
A^{1b} represents straight or branched optionally substituted C₁₋₁₂ alkylene;
R² represents —OR⁴ or, preferably, hydrogen, C₁₋₁₂ alkyl, C₁₋₁₂ hydroxyalkyl or C₁₋₁₂ haloalkyl;
R³ represents hydrogen or —OR⁴;
each R⁴ is independently selected from the group consisting of hydrogen, —R⁸—OR⁹, —R⁸—C(O)OR⁹, C₁₋₁₂ alkyl, and optionally substituted -A³-B³;
A³ represents a direct bond;
B³ represents cycloalkyl;
R⁵ represents hydrogen or —C(O)R⁹;
each R⁶ and R⁷ is independently selected from the group consisting of C₁₋₁₂ alkyl, halo, C₁₋₁₂ haloalkyl, —R¹¹—OR⁹, —R¹¹—CN, —R¹¹—NO₂, —R¹¹—C(O)OR⁹, —R¹¹—N(R⁹)R¹⁰ and —R¹¹—C(O)N(R⁹)R¹⁰;
each R⁹ and R¹⁰ is independently selected from the group consisting of hydrogen, C₁₋₁₂ alkyl, C₁₋₁₂ haloalkyl, C₁₋₁₂ haloalkenyl, -A⁵-O-A⁶ (e.g. —C₁₋₁₂ alkylene-O—C₁₋₁₂ alkyl) and optionally substituted -A⁷-B⁷;
A⁷ represents a direct bond or C₁₋₁₂ alkyl;
B⁷ represents cycloalkyl, aryl, heterocyclyl or heteroaryl;
each R¹¹ independently represents a direct bond or R⁸; and/or
each R⁸ independently represents straight or branched optionally substituted C₁₋₁₂ alkylene.

Compounds of the invention (and particularly those in which R¹ represents -A¹-T^{z}-B¹) that may be mentioned include those in which:

R² represents hydrogen, C₁₋₁₂ alkyl or C₂₋₁₂ alkenyl, which latter two groups are optionally substituted by one or more substituents selected from X¹;
B³ represents heterocyclyl (optionally substituted as defined herein) or, preferably, C₃₋₁₅ cycloalkyl (optionally substituted as defined herein);
each R⁶ and R⁷ independently represents halo, —R¹¹—OR⁹, —R¹¹—CN, —R¹¹—NO₂, —R¹¹—C(O)OR⁹, —R¹¹—N(R)R¹⁰, —R¹¹—C(O)N(R⁹)R¹⁰ and/or C₁₋₁₂ alkyl optionally substituted by one or more substituents selected from X⁵;
Z³ represents, on each occasion when used herein, G¹, ═O, -A^{x}-B^{y} and/or G²;
A¹, A⁴ and A⁵ independently represent C₁₋₁₂ alkylene optionally substituted by one or more substituents selected from X⁷;
A² and A⁷ independently represent a direct bond or C₁₋₁₂ alkylene optionally substituted by one or more substituents selected from X⁸;
Y¹ and Y², independently represent, on each occasion when used herein, G¹, G² and/or —R¹⁵—OR¹⁷—N(R¹⁴)₂;
T⁵ and T⁶ independently represent halo;
G¹ represents —NO₂ or, more preferably, halo, —CN, C₁₋₁₂ alkyl (optionally substituted by one or more substituents selected from T⁵) or C₂₋₁₂ alkenyl (optionally substituted by one or more substituents selected from T⁶);
when any of X¹, X², X³, X⁴, X⁵, X⁶, X⁷, X⁸, X⁹ and X¹⁰ represent aryl, C₃₋₁₅ cycloalkyl, heterocyclyl or heteroaryl, then such groups are optionally substituted as defined herein, or, more preferably unsubstituted;
when G¹ represents C₂₋₁₂ alkenyl, then it may be unsubstituted or substituted by one or more halo atoms;
when G¹ represents C₁₋₁₂ alkyl or C₂₋₁₂ alkenyl, then such groups may be optionally substituted as defined herein, or, are more preferably, unsubstituted;
when A^{x} represents C₁₋₁₂ alkylene, then this group may be optionally substituted as hereinbefore defined, but is preferably unsubstituted;
when B^{x} represents aryl or heteroaryl, then these groups may be optionally substituted as hereinbefore defined, but are preferably unsubstituted;
when B^{y} represents cycloalkyl or heterocyclyl then these groups may be optionally substituted as hereinbefore defined, but are preferably unsubstituted;
when R¹⁵ represents C₁₋₁₂ alkylene or C₂₋₁₂ alkenylene, then such groups may be optionally substituted as defined herein, or, are preferably unsubstituted;
each R¹⁶ independently represents, on each occasion when used herein, -A^{y1}-B^{y1} or C₁₋₁₂ alkyl optionally substituted by one or more halo atoms;
when R¹⁷ represents C₁₋₁₂ alkylene or C₂₋₁₂ alkenylene, then such groups may be optionally substituted as defined herein, or, are preferably unsubstituted;
when A^{x1} and A^{y1} represent C₁₋₁₂ (e.g. C₁₋₆) alkylene, then that group may be optionally substituted as defined herein, or, is preferably unsubstituted;
when B^{x1} and B^{y1} represent C₃₋₁₅ cycloalkyl, aryl, heterocyclyl or heteroaryl, then such groups may be optionally substituted as defined herein, or, are preferably unsubstituted;
when R¹⁴ represents C₁₋₁₂ alkyl, then such a group is preferably unsubstituted, and, when it is substituted, then it is preferably substituted by one or more halo atoms;
each R¹⁴ independently represents, on each occasion when used herein, hydrogen, -A^{x1}-B^{x1} or C₁₋₁₂ alkyl, which latter group may be unsubstituted or is substituted by one or more substituents selected from halo;
E¹ represents ═O, —OR¹⁸, or, more preferably, halo or —C(O)N(R¹⁸)₂;
R¹⁸ and R¹⁹ independently represent hydrogen;
T^{Z} represents a direct bond;
when R⁹ and R¹⁰, together with the nitrogen atom to which they are both attached, linked together to form an optionally substituted heterocyclyl or an optionally substituted heteroaryl group, then such groups are preferably 5- to 10-membered monocyclic or bicyclic groups, preferably containing one to three (e.g. one or two) heteroatoms selected from sulfur or, preferably nitrogen or oxygen.

Preferred compounds of the invention (in particular those in which R¹ represents -A¹-T^{z}-B¹) include those in which:

m represents 0, 1, or 2;
n represents 0, 1 or 2;
B¹ represents a bicyclic heterocyclyl group or, preferably, a bicyclic heteroaryl group, both of which are optionally substituted as defined herein;
R² represents hydrogen, C₁₋₁₂ (e.g. C₁₋₆) alkyl or C₁₋₁₂ (e.g. C₁₋₆) alkenyl, which latter two groups are optionally substituted by one or more substituents selected from X¹ (such as hydroxy or, preferably, halo);
R³ represents -A²-B² or, preferably, hydrogen or —OR⁴;
each R⁴ independently represents hydrogen, —R⁸—OR⁹, —R⁸—C(O)OR⁹, C₁₋₁₂ (e.g. C₁₋₆) alkyl (optionally substituted by one or more substituents selected from X³) or -A³-B³;
A² and A³ independently represent C₁₋₃ alkylene or, preferably, a direct bond;
B³ represents C₃₋₁₅ (e.g. C₆₋₁₀) cycloalkyl (optionally substituted by one or more substituents selected from Z³) or a 3- to 18- (e.g. 5- to 10-) membered heterocyclyl group (optionally substituted by one or more substituents selected from Z^{4a});
R⁵ represents -A⁴-B⁴ or, preferably, hydrogen or —C(O)R⁹;
each R⁶ and R⁷ independently represent halo, —R¹¹—OR⁹, —R¹¹—CN, —R¹¹—NO₂, —R¹¹—C(O)OR⁹, —R¹¹—N(R⁹)R¹⁰, —R¹¹—C(O)N(R⁹)R¹⁰ and/or C₁₋₁₂ (e.g. C₁₋₆) alkyl optionally substituted by one or more substituents selected from X⁵ (e.g. halo; so forming a haloalkyl group);
each R⁸ independently represents C₁₋₁₂ alkylene optionally substituted by one or more substituents selected from X¹⁰;
each R⁹ and R¹⁰ independently represent hydrogen, C₁₋₁₂ (e.g. C₁₋₆) alkyl, C₁₋₁₂ (e.g. C₁₋₆) alkenyl (which latter two groups are optionally substituted by one or more substituents selected from X⁶ (e.g. halo)), -A⁵-O-A⁶ and/or -A⁷-B⁷; or
R⁹ and R¹⁰ are linked together to form, together with the nitrogen atom to which they are necessarily attached, a 5- or 6-membered heterocyclyl group (e.g. pyrrolidinyl, piperidinyl, morpholinyl or piperazinyl), which is optionally substituted by one or more substituent selected from Z^{1a} (e.g. halo, —CH₃ and ═O);
A¹, A^{1a}, A^{1b}, A^{1c}, A^{1d}, A^{1e}, A⁴ A⁵ independently represent C₁₋₆ (e.g. C₁₋₃) alkylene (optionally substituted as defined herein, e.g. by one or more X⁷ substituents);
A⁶ represents C₁₋₆ (e.g. C₁₋₃) alkyl (optionally substituted as defined herein);
A², A³ and A⁷ independently represent a direct bond or C₁₋₆ (e.g. C₁₋₃) alkylene (optionally substituted as defined herein, e.g. by one or more X⁸ substituents);
X¹, X², X³, X⁴, X⁵, X⁶, X⁷, X⁸, X⁹, X¹⁰ independently represent a 5- or 6-membered heterocyclyl group (preferably containing a nitrogen heteroatom and optionally a further nitrogen or oxygen heteroatom; optionally substituted by one or more T³ substituents), —OR¹⁴, N(R¹⁴)₂, —C(O)OR¹⁴, —C(O)N(R¹⁴)₂, —S(O)ₜN(R¹⁴)₂ or more preferably, G¹;
t represents 2;
Y¹ and Y² independently represent G² or preferably, G¹;
Z^{1a}, Z^{1b}, Z^{2a}, Z^{2b}, Z³, Z^{4a} and Z^{4b} independently represent ═O or, preferably, G² or G¹;
G¹ represents —CN, —NO₂ or, preferably, halo or C₁₋₆ (e.g. C₁₋₃) alkyl optionally substituted by one or more T⁵ substituents (such as halo (e.g. fluoro) atoms);
G² represents —R¹⁵—N(R¹⁴)₂, C(O)OR¹⁴, —R¹⁵—C(O)N(R¹⁴)₂, —R¹⁵—S(O)ₜN(R¹⁴)₂ or more preferably —R¹⁵—O—R¹⁴;
T¹, T², T³, T⁴, T⁵, T⁶, T⁷ and T⁸ independently represent halo (e.g. chloro or fluoro) or C₁₋₃ alkyl optionally substituted by one or more Q^{x1} or halo substituents as appropriate);
Q^{x1} and Q^{x2} independently represent halo (e.g. chloro or fluoro);
R¹⁵ represents a direct bond;
R¹⁴ represents C₁₋₆ (e.g. C₁₋₃) alkyl (e.g. methyl) optionally substituted by one or more substituents selected from E¹ (e.g. —C(O)N(R¹⁸)₂ and halo (e.g. fluoro));
each R¹⁶ independently represents C₁₋₃ alkyl (optionally substituted by one or more fluoro atoms);
R¹⁷ represents C₁₋₆ (e.g. C₁₋₃) alkylene;
R¹⁸ and R¹⁹ independently represent hydrogen;
R^{w1} and R^{w2} independently represent C₁₋₃ alkyl optionally substituted by one or more halo atoms, or, more preferably represent hydrogen.

When B¹ represents a monocyclic heteroaryl group, preferred groups include optionally substituted (e.g. by Z²) imidazolyl (e.g. 2-imidazolyl), triazolyl (e.g. 1,2,4-triazolyl) or, preferably, pyridyl (e.g. 2-, 3- or 4-pyridyl), thienyl (e.g. 2-thienyl) and furanyl (e.g. 2-furanyl). When T^{z} represents a direct bond, then B¹ preferably represents optionally substituted pyridyl, thienyl or furanyl. When T^{z} represents a substituent other than a direct bond (e.g. —N(H)— or —C(O)N(H)—), then B¹ preferably represents optionally substituted imidazolyl or triazolyl.

When B¹ represents a polycyclic (e.g. bicyclic) heteroaryl group, preferred groups include optionally substituted (e.g. by Z²) 1,3-dihydroindol-2-one-yl (e.g. 1,3-dihydroindol-2-one-3-yl), 2,3-dihydrobenzo[1,4]dioxinyl (e.g. 2,3-dihydrobenzo[1,4]dioxin-2-yl), benzo[1,4]oxazinyl (e.g. benzo[1,4]oxazin-3-yl), pyrrolopyridinyl (e.g. pyrrolo[2,3-b]pyridin-2-yl), imidazopyridyl, thiazolopyridyl, or, more preferably, benzoxazolyl (e.g. 2-benzoxazolyl), benzimidazolyl (e.g. 2-benzimidazolyl), benzofuranyl (e.g. 2-benzofuranyl), indolyl (e.g. 3-indolyl), benzothienyl (e.g. 3-benzothienyl), benzothiazolyl (e.g. 2-benzothiazolyl), benzotriazolyl (e.g. benzo-1, 2-3-triazol-1-yl) and oxazolopyridinyl (e.g. oxazolo[5,4-b]pyridinyl, oxazolo[5,4-c]pyridinyl, oxazolo[4,5-b]pyridinyl or oxazolo[4,5-c]pyridinyl). Particularly preferred bicyclic heteroaryl groups include benzimidazolyl (e.g. 2-benzimidazolyl) or, preferably, benzoxazolyl (e.g. 2-benzoxazolyl), benzofuranyl (e.g. 2-benzofuranyl) and oxazolopyridinyl (e.g. oxazolo[5,4-b]pyridinyl, oxazolo[5,4-c]pyridinyl, oxazolo[4,5-b]pyridinyl or oxazolo[4,5-c]pyridinyl) groups.

Optional substituents on such B¹ groups include C₁₋₄ alkyl (e.g. methyl) optionally substituted by one or more halo atoms (so forming, for example, a difluoromethyl or trifluoromethyl group); halo (e.g. chloro or fluoro); —CN; and —O—C₁₋₄ alkyl (e.g. methoxy) optionally substituted by one or more substituents selected from —C(O)N(R¹⁸)₂ (in which R¹⁸ is preferably hydrogen; so forming, for example an acetamidoxy substituent) or, more preferably, halo (so forming, for example, a difluoromethoxy or trifluoromethoxy group). Particularly preferred such substituents include fluoro atoms. Further, such substituents may, for example, when substituted on a benzimidazolyl (e.g. 2-benzimidazolyl) or benzoxazolyl (e.g. 2-benzoxazolyl) group, be in the 4- to 7- (e.g. 4-, 7- or, preferably, 5-) position.

Particularly preferred compounds of the invention (for example those in which R¹ represents -A¹-T^{Z}-B¹) that may be mentioned include those in which: m and n independently represent 0;

R¹ represents hydrogen; C₁₋₄ (e.g. C₁₋₂) alkyl (such as methyl) optionally substituted by one or more substituents selected from X¹; C₂₋₄ (e.g. C₂₋₃) alkenyl (such as allyl, i.e. —CH₂—CH═CH₂), -A^{1a}-N(R⁹)R¹⁰, -A^{1b}-OR⁹, -A^{1c}-C(O)R⁹, -A^{1d}-C(O)OR⁹, -A^{1e}-C(O)N(R⁹)R¹⁰ or, preferably, -A¹-T^{z}-B¹;
R² represents C₁₋₄ alkyl, C₂₋₄ (e.g. C₂₋₃) alkenyl (e.g. allyl), both of which latter two groups are optionally substituted by one or more X¹ groups, or, R² preferably represents hydrogen; or
R¹ and R² together form a ═C(R⁹)R¹⁹ group;
R³ represents -A²-B² or, preferably, —OR⁴;
R⁴ represents -A³-B³ or a C₁₋₆ alkyl group, for instance a straight-chain alkyl (e.g. isopropyl, n-propyl, ethyl or, preferably, methyl) group optionally substituted by one or more substituents selected from X³, for example —OR¹⁴, —C(O)OR¹⁴ or, preferably, fluoro (so forming, for example, a difluoromethyl or trifluoromethyl group);
X¹ represents —OR¹⁴ (in which R¹⁴ preferably represents hydrogen);
X³ represents —OR¹⁴ (in which R¹⁴ preferably represents hydrogen), —C(O)OR¹⁴ (in which R¹⁴ preferably represents hydrogen or C₁₋₂ alkyl) or, preferably, G¹;
A² represents a direct bond;
A³ represents a C₁₋₂ methylene (e.g. —CH₂—) or, preferably, direct bond;
B² represents aryl (e.g. phenyl), which group is optionally substituted by one or more substituents selected from Y¹, but is preferably unsubstituted;
B³ represents a C₃₋₅ cycloalkyl (e.g. a C₃ cyclopropyl group or, preferably a C₅ cyclopentyl group) or a 4- to 6- (e.g. a 5- or 6-) membered heterocyclyl group (e.g. a 4- or, preferably, a 5-membered heterocyclyl group containing one oxygen atom, such as oxetanyl, e.g. 3-oxetanyl, or, more preferably, tetrahydrofuranyl, e.g. 3-tetrahydrofuranyl, group);
R⁵ represents -A⁴-B⁴, —C(O)R⁹ or, preferably, hydrogen;
each R⁶ and R⁷ independently represent halo or C₁₋₁₂ (e.g. C₁₋₆) alkyl optionally substituted by one or more substituents selected from X⁵ (e.g. halo; so forming a haloalkyl group);
A^{1a} represents C₁₋₃ (e.g. C₁₋₂) alkylene, such as ethylene or methylene (i.e. —CH₂—);
A^{1b} represents C₁₋₄ (e.g. C₁₋₃) alkylene (such as propylene, ethylene or methylene) optionally substituted by one or more (e.g. one) substituent(s) selected from X⁷;
A^{1c}, A^{1d} and A^{1e} independently represent C₁₋₂ alkylene (e.g. methylene);
A¹ represents C₁₋₃ (e.g. C₁₋₂) alkylene, such as ethylene or, preferably, methylene (which group is optionally substituted by one or more halo, e.g. fluoro, atoms, or, preferably unsubstituted);
T^{Z} represents —N(H)—, —C(O)N(H)— or, preferably, a direct bond;
when B¹ represents heterocyclyl, then it is preferably a monocyclic 5- or 6-membered ring containing two or, preferably, one heteroatom (e.g. nitrogen or preferably oxygen), so forming for example a tetrahydrofuranyl group (e.g. 3-tetrahydrofuranyl);
A¹ and T^{z} together represent —CH₂—N(H)—, —CH₂—CH₂—N(H)—, —CH₂—C(O)—N(H) or, preferably, —CH₂—;
A⁴ represents a direct bond or, preferably, C₁₋₂ alkylene (e.g. methylene);
B⁴ represents aryl (e.g. phenyl), which group is preferably unsubstituted;
R⁹ represents hydrogen, C₁₋₁₂ (e.g. C₁₋₆, such as C₁₋₃) alkyl (e.g. methyl; optionally substituted by one or more, e.g. one, substituent(s) selected from X⁶) or, preferably, -A⁷-B⁷;
R¹⁰ represents hydrogen or -A⁷-B⁷;
X⁶ represents aryl (e.g. phenyl), which group is optionally substituted by one or more T¹ substituents, but is preferably unsubstituted;
X⁷ represents —OR¹⁴ (in which R¹⁴ preferably represents hydrogen);
A⁷ represents a direct bond or C₁₋₃ (e.g. C₁₋₂) alkylene (e.g. methylene);
B⁷ represents aryl (e.g. phenyl; optionally substituted by one or more substituents selected from Y¹), heteroaryl (e.g. triazolyl, imidazolyl, pyridyl, quinolinyl, benzodioxolyl or furanyl; which heteroaryl group(s) is/are optionally substituted by one or more substituents selected from Z^{4b}) or heterocyclyl (e.g. piperidinyl; optionally substituted by one or more substituents selected from Z^{4a});
Y¹ represents G¹ or G²;
Z^{4a} represents G² or -A^{x}-B^{y};
Z^{4b} represents G¹;
G¹ represents —CN, —NO₂, halo (e.g. fluoro or chloro), C₁₋₄ alkyl (e.g. tert-butyl, isopropyl or methyl), which alkyl group is optionally substituted by one or more substituents selected from r;
G² represents -A^{x}-B^{x}, —R¹⁵—OR¹⁴ or —R¹⁵—N(R¹⁴)₂;
R¹⁵ represents a direct bond;
R¹⁴ represents hydrogen, -A^{x1}-B^{x1} or C₁₋₄ (e.g. C₁₋₂) alkyl (e.g. n-butyl, methyl or ethyl), which alkyl group is optionally substituted by one or more substituents selected from E¹;
A^{x} and A^{x1} independently represent a direct bond;
B^{x} represents aryl (e.g. phenyl) or, preferably, heteroaryl (e.g. imidazolyl, such as 4-imidazol-1-yl), both of which are optionally substituted as defined herein, but are more preferably unsubstituted;
B^{y} represents heterocyclyl (e.g. a 4- to 6-membered heterocyclyl group containing one or two heteroatoms preferably selected from oxygen or, more particularly, nitrogen, so forming for example a pyrrolidinyl or imidazolyl group);
B^{x1} represents aryl (e.g. phenyl) optionally substituted by one or more halo (e.g. chloro) atoms;
T⁵ represents halo (e.g. fluoro) or —OC₁₋₆ alkyl (e.g. —OC₁₋₃ alkyl, such as —OCH₂CH₃; which alkoxy group is preferably unsubstituted);
E¹ represents halo (e.g. fluoro) or —N(R¹⁸)₂;
R¹⁸ represents methyl.

Further preferred compounds of the invention include those in which:

R² represents hydrogen, allyl or —CH₂OH;
R³ represents —OR⁴, in which R⁴ preferably represents C₁₋₄ alkyl (e.g. methyl), C₃₋₆ cycloalkyl (e.g. cyclopropyl or cyclopentyl), a 4- to 6-membered heterocyclyl group (e.g. oxetanyl or, preferably, tetrahydrofuranyl), which cycloalkyl and heterocyclyl groups may be attached via a C₁₋₂ alkylene linker group (e.g. methylene; so forming, for example, a cyclopropylmethyl group);
when R¹ represents C₁₋₁₂ (e.g. C₁₋₄) alkyl, C₂₋₁₂ (e.g. C₂₄) alkenyl or C₂₋₁₂ alkynyl, all of which are optionally substituted as defined herein, then such groups preferably represent methyl or allyl;
when R¹ represents -A^{1a}-N(R)R¹⁰, then such groups preferably represent —(CH₂)₂—N(R⁹)R¹⁰ or —(CH₂)—N(R⁹)R¹⁰, in which R⁹ and R¹⁰ are as defined herein, and R⁹ preferably represents C₁₋₁₂ (e.g. C₁₋₆, such as C₁₋₃) alkyl (e.g. methyl; optionally substituted by one or more, e.g. one, substituent(s) selected from X⁶) or, preferably, -A⁷-B⁷ (e.g. benzyl, heteroaryl (e.g. 1,2,4-triazol-4-yl) or aryl (e.g. phenyl optionally substituted as defined herein, e.g. by one or more Y¹ substituents));
when R¹ represents -A^{1b}-OR⁹, then R⁹ preferably represents hydrogen and such R¹ groups preferably represent —CH₂—OH or —CH₂—C(H)(OH)—CH₂—OH;
when R¹ represents -A^{1c}-C(O)R⁹, then R⁹ preferably represents hydrogen or -A⁷-B⁷, and such R¹ groups are preferably —CH₂—C(O)-[4-(pyrrolidin-1-yl)piperidin-1-yl] or —CH₂—C(O)H;
when R¹ represents -A^{1d}-C(O)OR⁹, then R⁹ preferably represents C₁₋₆ alkyl, or, more particularly, hydrogen or -A⁷-B⁷, in which A⁷ preferably represents a direct bond, B⁷ represents heterocyclyl (e.g. 1-piperidinyl; optionally substituted by one or more substituents selected from Z^{4a}) and/or Z^{4a} represents -A^{x}-B^{y};
when R¹ represents -A^{1e}-C(O)N(R⁹)R¹⁰, then R⁹ preferably represents -A⁷-B⁷; A⁷ represents a direct bond; B⁷ represents aryl (e.g. phenyl) or heteroaryl, both of which are optionally substituted as defined herein;
when R⁵ represents —C(O)R⁹, then: R⁹ represents -A⁷-B⁷ or C₁₋₃ (e.g. C₁₋₂) alkyl (e.g. methyl); A⁷ represents a direct bond; and/or B⁷ represents aryl (e.g. phenyl).

When R¹ represents -A^{1a}-N(R⁹)R¹⁰, then the preferred R⁹ groups (e.g. when R⁹ represents C₁₋₃ alkyl substituted by X⁶ or, R⁹ preferably represents A⁷-B⁷) include 4-cyanophenyl, 1,2,4-triazol-4-yl, 4-fluorophenyl, 3-fluorophenyl, 2-fluorophenyl, unsubstituted phenyl, 4-isopropylphenyl, 4-chlorophenyl, 3-methylphenyl, 3,4-dimethoxyphenyl, 3-phenoxyphenyl, 3,5-dimethoxyphenyl, 4-methoxyphenyl, benzo[1,3]dioxol-5-yl, 4-tert-butylphenyl, 3,4-difluorophenyl, 4-pyridyl, 4-methylphenyl, 2-methylphenyl, 2,4-dimethylphenyl, 3-ethoxyphenyl, 4-ethoxyphenyl, 3-nitrophenyl and benzyl.

When R¹ represents -A^{1e}-C(O)N(R⁹)R¹⁰, then the preferred R⁹ groups (e.g. when R⁹ represents C₁₋₃ alkyl substituted by X⁶ or, R⁹ preferably represents A⁷-B⁷) include 2-imidazolyl, 3-pyridyl, 4-fluorophenyl, unsubstituted phenyl, 4-(imidazol-1-yl)phenyl, 6-quinolinyl and 4-pyridyl.

When R¹ and R² together form ═C(R⁹)R¹⁰, then R⁹ preferably represents unsubstituted phenyl, 4-fluorophenyl, 2,4-difluorophenyl, 3-fluorophenyl, 3-phenoxyphenyl, 6-methylpyrid-2-yl, 4-(imidazol-1-yl)phenyl, 3-methoxyphenyl, 3-trifluoromethoxyphenyl, 4-(diethylamino)phenyl, 2-methoxyphenyl, 4-(diethoxymethyl)phenyl, 4-(3-(dimethylamino)propoxy)phenyl, 3,4-dimethoxyphenyl, 2,5-difluorophenyl, 2-methylphenyl, 2-chlorophenyl, 2-nitrophenyl, 3-methylphenyl, 3-trifluoromethylphenyl, 3-chlorophenyl, benzo[1,3]dioxol-4-yl, benzo[1,3]dioxol-5-yl, 2-chloro-5-trifluoromethylphenyl, 3,5-dichlorophenyl, 3-furanyl, 2-pyridyl, 3-pyridyl, 4-pyridyl, 3-(4-chlorophenoxy)phenyl, 2,3-difluorophenyl, 4-chlorophenyl, 2-trifluoromethyl-4-fluorophenyl, 3-fluoro-4-methoxyphenyl, 3,5-bis(trifluoromethyl)phenyl, 3,4,5-trimethoxyphenyl, 4-phenoxyphenyl, 2,6-dimethoxyphenyl, 2-fluorophenyl, 4-trifluoromethoxyphenyl, 4-isopropylphenyl and 4-n-butyloxyphenyl.

Preferred Y¹, Y², Z^{1a}, Z^{1b}, Z^{2a}, Z^{2b}, Z³, Z^{4a} and Z^{4b} (e.g. A^{1b}, Z^{2a}, Z^{4b} and, especially Y¹) substituents (e.g. when B⁷ represents aryl substituted by one or more substituents selected from Y¹) include: halo (e.g. fluoro, chloro, bromo or iodo);

### —CN;

### —NO₂;

C₁₋₆ alkyl, which alkyl group may be cyclic, part-cyclic, unsaturated or, is preferably, linear or branched (e.g. C₁₋₄ alkyl (such as methyl, ethyl, n-propyl, isopropyl, butyl (e.g. t-butyl)), all of which are optionally substituted with one or more groups selected from halo (e.g. fluoro; so forming, for example, a fluoromethyl, difluoromethyl or, preferably, trifluoromethyl group) and —OR²⁰ (in which R²⁰ represents hydrogen or C₁₋₃ alkyl (e.g. methyl or, preferably ethyl; so forming for example a diethoxymethyl group); aryl (e.g. phenyl) or heteroaryl (e.g. a 5- or 6-membered heteroaryl group preferably containing one or two heteroatoms selected from oxygen or, preferably nitrogen; so forming for example an imidazol-1-yl group);

### —OR²¹;

### —C(O)OR²²;

-J¹-N(R²³)R²⁴ (in which J¹ represents —C(O)—, preferably, —S(O)₂ or, more preferably a direct bond);
wherein R²¹, R²², R²³ and R²⁴ independently represent:
hydrogen;
C₁₋₄ (e.g. C₁₋₂) alkyl (e.g. methyl, ethyl, propyl (e.g. n-propyl or isopropyl) or butyl (e.g. n-butyl or t-butyl)), (which alkyl group is optionally substituted by one or more substituents selected from halo (e.g. fluoro; so forming e.g. a trifluoromethyl group), C₃₋₆ cycloalkyl (e.g. cyclopropyl, so forming for example a cyclopropylmethyl group; optionally substituted by one or more substituents selected from C₁₋₃ alkyl and halo, such as methyl and fluoro), aryl (e.g. phenyl, so forming for example a benzyl group; optionally substituted by one or more substituents selected from C₁₋₃ alkyl and halo, such as methyl and fluoro), —N(R²⁵)R²⁶ or —C(O)N(R²⁷)₂, in which R²⁵, R²⁶ and each R²⁷ independently represent hydrogen or C₁₋₃ alkyl, such as methyl; so forming for example a dimethylaminopropoxy or acetamidoxy group);
C₃₋₆ cycloalkyl (e.g. cyclopropyl; optionally substituted by one or more substituents selected from C₁₋₃ alkyl and halo, such as methyl and fluoro);
aryl (e.g. phenyl; optionally substituted by one or more halo, e.g. chloro, atoms); or
R²³ and R²⁴ may be linked together to form, together with the nitrogen atom to which they are necessarily attached, a 5- or 6-membered heterocyclyl group optionally containing a further heteroatom preferably selected from oxygen and nitrogen (so forming, for example, a piperazinyl, morpholinyl, piperidinyl or pyrrolidinyl group), and which is optionally substituted by one or more substituents selected from C₁₋₃ alkyl and halo, such as methyl and fluoro.

Particularly preferred Y¹, Y², Z^{1a}, Z^{1b}, Z^{2a}, Z^{2b}, Z³, Z^{4a} and Z^{4b} (e.g. Z^{1b}, Z^{2a}, Z^{4b} and, especially Y¹) substituents include —CN, —NO₂, fluoro, chloro, tert-butyl, isopropyl, methyl, trifluoromethyl, difluoromethyl; fluoromethyl, hydroxy, methoxy, trifluoromethoxy, difluoromethoxy, fluoromethoxy, ethoxy, cyclopropyloxy, cyclopropylmethoxy, n-butyloxy, phenoxy, chlorophenoxy (e.g. 4-chlorophenoxy), benzyloxy, 3-(dimethylamino)propoxy, acetamidoxy, amino, diethoxymethyl, diethylamino, dimethylamino, methylamino, ethylamino, ethylmethylamino and imidazol-1-yl.

Further preferred compounds of the invention (for example those in which R¹ represents -A¹-T^{z}-B¹) include those in which:

for example, when B¹ represents a bicyclic heteroaryl group, then the R¹ substituent at 3-position of the essential piperidin-2-one ring is in the (R)-configuration;
for example, when R³ represents —OR⁴ in which R⁴ represents -A³-B³, A³ represents a direct bond and B³ represents a 3-tetrahydrofuranyl group, then the chiral atom of that group, is preferably in the (R)-configuration.

Particularly preferred compounds of the invention, for example when R¹ represents -A¹-T^{Z}-B¹, in which A¹ represents C₁₋₁₂ alkylene and B¹ represents heteroaryl (and T^{z} preferably represents a direct bond), include those in the following list:
(3R,5S)-3-(benzo[d][1,3]dioxol-5-ylmethyl)-5-(3-(cyclopentyloxy)-4-methoxyphenyl)piperidin-2-one;
(3S,5S)-3-(benzo[d][1,3]dioxol-5-ylmethyl)-5-(3-(cyclopentyloxy)-4-methoxyphenyl)piperidin-2-one; preferably,
(3R,5S)-5-(3-cyclopentyloxy-4-methoxyphenyl)-3-(7-methoxy-2-benzofurylmethyl)piperidin-2-one;
(3S,5S)-5-(3-cyclopentyloxy-4-methoxyphenyl)-3-(7-methoxy-2-benzofurylmethyl)piperidin-2-one;
(3R,5S)-5-(3-cyclopentyloxy-4-methoxyphenyl)-3-(7-fluoro-2-benzofurylmethyl)piperidin-2-one;
(3S,5S)-5-(3-cyclopentyloxy-4-methoxyphenyl)-3-(7-fluoro-2-benzofurylmethyl)piperidin-2-one; more preferably (3R,5S)-3-(2-benzoxazolylmethyl)-5-(3-cyclopentyloxy-4-methoxyphenyl)piperidin-2-one;
(3S,5S)-3-(2-benzoxazolylmethyl)-5-(3-cyclopentyloxy-4-methoxyphenyl)piperidin-2-one;
(3R,5S)-5-(3-cyclopentyloxy-4-methoxyphenyl)-3-(5,6-dimethyl-2-benzimidazolylmethyl)piperidin-2-one;
(3S,5S)-5-(3-cyclopentyloxy-4-methoxyphenyl)-3-(5,6-dimethyl-2-benzimidazolylmethyl)piperidin-2-one;
(3R,5S)-5-(3-cyclopentyloxy-4-methoxyphenyl)-3-(3-pyridinylmethyl)piperidin-2-one;
(3S,5S)-5-(3-cyclopentyloxy-4-methoxyphenyl)-3-(3-pyridinylmethyl)piperidin-2-one;
(3R,5S)-3-(2-benzofurylmethyl)-5-(3-cyclopentyloxy-4-methoxyphenyl)piperidin-2-one;
(3S,5S)-3-(2-benzofurylmethyl)-5-(3-cyclopentyloxy-4-methoxyphenyl)piperidin-2-one;
(3R,5S)-5-(3-cyclopentyloxy-4-methoxyphenyl)-3-(2-thienylmethyl)piperidin-2-one;
(3S,5S)-5-(3-cyclopentyloxy-4-methoxyphenyl)-3-(2-thienylmethyl)piperidin-2-one;
(3R,5S)-5-(3-cyclopentyloxy-4-methoxyphenyl)-3-(2-furylmethyl)piperidin-2-one;
(3S,5S)-5-(3-cyclopentyloxy-4-methoxyphenyl)-3-(2-furylmethyl)piperidin-2-one;
(3S,5S)-5-(3-cyclopentyloxy-4-methoxyphenyl)-3-(3-indolylmethyl)piperidin-2-one;
(3R,5S)-3-(3-benzothienylmethyl)-5-(3-cyclopentyloxy-4-methoxyphenyl)piperidin-2-one;
(3S,5S)-3-(3-benzothienylmethyl)-5-(3-cyclopentyloxy-4-methoxyphenyl)piperidin-2-one;
(3R,5S)-3-(2-benzothiazolylmethyl)-5-(3-cyclopentyloxy-4-methoxyphenyl)piperidin-2-one;
(3S,5S)-3-(2-benzothiazolylmethyl)-5-(3-cyclopentyloxy-4-methoxyphenyl)piperidin-2-one;
(3R,5S)-5-(3-cyclopentyloxy-4-methoxyphenyl)-3-(5-methyl-2-benzoxazolylmethyl)piperidin-2-one;
(3S,5S)-5-(3-cyclopentyloxy-4-methoxyphenyl)-3-(5-methyl-2-benzoxazolylmethyl)piperidin-2-one;
(3R,5S)-3-(6-chloro-2-benzoxazolylmethyl)-5-(3-cyclopentyloxy-4-methoxyphenyl)piperidin-2-one;
(3S,5S)-3-(6-chloro-2-benzoxazolylmethyl)-5-(3-cyclopentyloxy-4-methoxyphenyl)piperidin-2-one;
(3R,5S)-5-(3-cyclopentyloxy-4-methoxyphenyl)-3-(4-methyl-2-benzoxazolylmethyl)piperidin-2-one;
(3S,5S)-5-(3-cyclopentyloxy-4-methoxyphenyl)-3-(4-methyl-2-benzoxazolylmethyl)piperidin-2-one;
(3R,5S)-5-(3-cyclopentyloxy-4-methoxyphenyl)-3-(6-methyl-2-benzoxazolylmethyl)piperidin-2-one;
(3S,5S)-5-(3-cyclopentyloxy-4-methoxyphenyl)-3-(6-methyl-2-benzoxazolylmethyl)piperidin-2-one;
(3R,5S)-3-(1-benzotriazolylmethyl)-5-(3-cyclopentyloxy-4-methoxyphenyl)piperidin-2-one;
(3S,5S)-3-(1-benzotriazolylmethyl)-5-(3-cyclopentyloxy-4-methoxyphenyl)piperidin-2-one;
(3R,5S)-5-(3-cyclopentyloxy-4-methoxyphenyl)-3-(5-nitro-2-benzoxazolylmeth-yl)piperidin-2-one;
(3S,5S)-5-(3-cyclopentyloxy-4-methoxyphenyl)-3-(5-nitro-2-benzoxazolylmethyl)piperidin-2-one;
(3R,5S)-3-(5-cyano-2-benzoxazolylmethyl)-5-(3-cyclopentyloxy-4-methoxyphenyl)piperidin-2-one
(3S,5S)-3-(5-cyano-2-benzoxazolylmethyl)-5-(3-cyclopentyloxy-4-methoxyphenyl)piperidin-2-one
(3R,5S)-5-(3-cyclopentyloxy-4-methoxyphenyl)-3-(5-fluoro-2-benzoxazolylmethyl)piperidin-2-one;
(3S,5S)-5-(3-cyclopentyloxy-4-methoxyphenyl)-3-(5-fluoro-2-benzoxazolylmethyl)piperidin-2-one;
(3R,5S)-5-(3-cyclopentyloxy-4-methoxyphenyl)-3-(5-trifluoromethyl-2-benzoxazolylmethyl)piperidin-2-one;
(3S,5S)-5-(3-cyclopentyloxy-4-methoxyphenyl)-3-(5-trifluoromethyl-2-benzoxazolylmethyl)piperidin-2-one;
(3S,5S)-5-(3-cyclopentyloxy-4-methoxyphenyl)-3-(7-methyl-2-benzoxazolylmethyl)piperidin-2-one;
(3R,5S)-5-(3-cyclopentyloxy-4-methoxyphenyl)-3-(7-methyl-2-benzoxazolylmethyl)piperidin-2-one;
(3S,5S)-5-(3-cyclopentyloxy-4-methoxyphenyl)-3-(5-methoxy-2-benzoxazolylmethyl)piperidin-2-one;
(3R,5S)-5-(3-cyclopentyloxy-4-methoxyphenyl)-3-(5-methoxy-2-benzoxazolylmethyl)piperidin-2-one;
(3S,5S)-5-(3-cyclopentyloxy-4-methoxyphenyl)-3-(4-methoxy-2-benzoxazolylmethyl)piperidin-2-one;
(3R,5S)-5-(3-cyclopentyloxy-4-methoxyphenyl)-3-(4-methoxy-2-benzoxazolylmethyl)piperidin-2-one;
(3S,5S)-5-(3-cyclopentyloxy-4-methoxyphenyl)-3-(4-fluoro-2-benzoxazolylmethyl)piperidin-2-one;
(3R,5S)-5-(3-cyclopentyloxy-4-methoxyphenyl)-3-(4-fluoro-2-benzoxazolylmethyl)piperidin-2-one;
(3S,5S)-5-(3-cyclopentyloxy-4-methoxyphenyl)-3-(7-fluoro-2-benzoxazolylmethyl)piperidin-2-one;
(3R,5S)-5-(3-cyclopentyloxy-4-methoxyphenyl)-3-(7-fluoro-2-benzoxazolylmethyl)piperidin-2-one;
(3S,5S)-3-(4-cyano-2-benzoxazolylmethyl)-5-(3-cyclopentyloxy-4-methoxyphenyl)piperidin-2-one
(3R,5S)-3-(4-cyano-2-benzoxazolylmethyl)-5-(3-cyclopentyloxy-4-methoxyphenyl)piperidin-2-one
(3S,5S)-3-(7-chloro-2-benzoxazolylmethyl)-5-(3-cyclopentyloxy-4-methoxyphenyl)piperidin-2-one;
(3R,5S)-3-(7-chloro-2-benzoxazolylmethyl)-5-(3-cyclopentyloxy-4-methoxyphenyl)piperidin-2-one;
(3S,5S)-5-(3-cyclopentyloxy-4-methoxyphenyl)-3-(4-trifluoromethyl-2-benzoxazolylmethyl)piperidin-2-one;
(3R,5S)-5-(3-cyclopentyloxy-4-methoxyphenyl)-3-(4-trifluoromethyl-2-benzoxazolylmethyl)piperidin-2-one;
(3S,5S)-5-(3-cyclopentyloxy-4-methoxyphenyl)-3-(5-methoxy-2-benzofurylmethyl)piperidin-2-one;
(3R,5S)-5-(3-cyclopentyloxy-4-methoxyphenyl)-3-(5-methoxy-2-benzofurylmethyl)piperidin-2-One;
(3R,5S)-5-(3-cyclopentyloxy-4-methoxyphenyl)-3-(6-methyl-2-pyridinylmethyl)piperidin-2-one;
(3S,5S)-5-(3-cyclopentyloxy-4-methoxyphenyl)-3-(6-methyl-2-pyridinylmethyl)piperidin-2-one;
(3R,5S)-5-(3-cyclopentyloxy-4-methoxyphenyl)-3-(2-pyridinylmethyl)piperidin-2-one;
(3S,5S)-5-(3-cyclopentyloxy-4-methoxyphenyl)-3-(2-pyridinylmethyl)piperidin-2-one;
(3R,5S)-5-(3-cyclopentyloxy-4-methoxyphenyl)-3-(4-pyridinylmethyl)piperidin-2-one; and
(3S,5S)-5-(3-cyclopentyloxy-4-methoxyphenyl)-3-(4-pyridinylmethyl)piperidin-2-one.

Further preferred compounds of the invention (for example when R¹ represents -A¹-T^{z}-B¹) that may be mentioned include:
(3R,5S)-3-(2-benzimidazolylmethyl)-5-(3-cyclopentyloxy-4-methoxyphenyl)piperidin-2-one;
(3S,5S)-3-(2-benzimidazolylmethyl)-5-(3-cyclopentyloxy-4-methoxyphenyl)piperidin-2-one;
(3R,5S)-5-(3-cyclopentyloxy-4-methoxyphenyl)-3-(1-methyl-2-benzimidazolylmethyl)piperidin-2-one;
(3S,5S)-5-(3-cyclopentyloxy-4-methoxyphenyl)-3-(1-methyl-2-benzimidazolylmethyl)piperidin-2-one;
(3R,5S)-5-(3-cyclopentyloxy-4-methoxyphenyl)-3-(5,6-dichloro-2-benzimidazolylmethyl)piperidin-2-one;
(3S,5S)-5-(3-cyclopentyloxy-4-methoxyphenyl)-3-(5 , 6-dichloro-2-benzimidazolylmethyl)piperidin-2-one.

Yet further preferred compounds of the invention (for example when R¹ represents -A¹-T^{z}-B¹) that may be mentioned include:
(3R,5S)-3-(4-acetamidoxy-2-benzoxazolylmethyl)-5-(3-cyclopentyloxy-4-methoxyphenyl)piperidin-2-one;
(3S,5S)-3-(4-acetamidoxy-2-benzoxazolylmethyl)-5-(3-cyclopentyloxy-4-methoxyphenyl)piperidin-2-one;
(3R,5S)-3-(7-acetamidoxy-2-benzoxazolyl methyl)-5-(3-cyclopentyloxy-4-methoxyphenyl)piperidin-2-one;
(3S,5S)-3-(7-acetamidoxy-2-benzoxazolylmethyl)-5-(3-cyclopentyloxy-4-methoxyphenyl)piperidin-2-one;
(3S,5S)-3-(4-cyano-2-benzoxazolylmethyl)-5-(3-cyclopentyloxy-4-difluoromethoxyphenyl)piperidin-2-one;
(3R,5S)-3-(4-cyano-2-benzoxazolylmethyl)-5-(3-cyclopentyloxy-4-difluoromethoxyphenyl)piperidin-2-one;
(3S,5S)-3-(4-cyano-2-benzoxazolylmethyl)-5-(3-cyclopentyloxy-4-trifluoromethoxyphenyl)piperidin-2-one;
(3R,5S)-3-(4-cyano-2-benzoxazolylmethyl)-5-(3-cyclopentyloxy-4-trifluoromethoxyphenyl)piperidin-2-one;
(3R,5S)-3-(4-cyano-2-benzoxazolylmethyl)-5-[4-difluoromethoxy-34(R)-3-tetrahydrofuranyloxy)phenyl]piperidin-2-one ;
(3S,5S)-3-(4-cyano-2-benzoxazolylmethyl)-5-[4-difluoromethoxy-34(R)-3-tetrahydrofuranyloxy)phenyl]piperidin-2-one;
(3R,5S)-3-(4-cyano-2-benzoxazolylmethyl)-5-[4-difluoromethoxy-34(S)-3-tetrahydrofuranyloxy)phenyl]piperidin-2-one;
(3S,5S)-3-(4-cyano-2-benzoxazolylmethyl)-5-[4-difluoromethoxy-34(S)-3-tetrahydrofuranyloxy)phenyl]piperidin-2-one;
(3R,5S)-3-(4-cyano-2-benzoxazolylmethyl)-5-[4-methoxy-3-((R)-3-tetrahydrofuranyloxy)phenyl]piperidin-2-one;
(3S,5S)-3-(4-cyano-2-benzoxazolylmethyl)-5-[4-methoxy-3-((R)-3-tetrahydrofuranyloxy)phenyl]piperidin-2-one;
(3R,5S)-3-(4-cyano-2-benzoxazolylmethyl)-5-[4-trifluoromethoxy-3-((R)-3-tetrahydrofuranyloxy)phenyl]piperidin-2-one;
(3S,5S)-3-(4-cyano-2-benzoxazolylmethyl)-5-[4-trifluoromethoxy-3-((R)-3-tetrahydrofuranyloxy)phenyl]piperidin-2-one;

Yet further compounds of the invention (for example when R¹ represents -A¹-T^{Z}-B¹) that may be mentioned include:
(3R,5S)-5-(3-cyclopentyloxy-4-methoxyphenyl)-3-(4-methyl-2-benzimidazolylaminomethyl)piperidin-2-one;
(3S,5S)-5-(3-cyclopentyloxy-4-methoxyphenyl)-3-(4-methyl-2-benzimidazolylaminomethyl)piperidin-2-one;
2-[(3R,5S)-5-(3-cyclopentyloxy-4-methoxyphenyl)-2-oxo-3-piperidinyl]-N-(2-imidazolyl)acetamide;
2-[(3S,5S)-5-(3-cyclopentyloxy-4-methoxyphenyl)-2-oxo-3-piperidinyl]-N-(2-imidazolyl)acetamide;
2-[(5S)-5-(3-cyclopentyloxy-4-methoxyphenyl)-2-oxo-3-piperidinyl]-N-(3-pyridinyl)acetamide;
2-[(5S)-5-(3-cyclopentyloxy-4-methoxyphenyl)-2-oxo-3-piperidinyl]-N-(4-pyridinyl)acetamide; and
(5S)-5-(3-cyclopentyloxy-4-methoxyphenyl)-3-[2-(1,2,4-triazol-4-ylamino)ethyl)piperidin-2-one.

Particularly preferred compounds of the invention, for example when R¹ represents hydrogen, include:
(S)-5-(3-(heptyloxy)-4-methoxyphenyl)piperidin-2-one;
(S)-5-(6-methoxybiphenyl-3-yl)piperidin-2-one;
(S)-5-(3-ethoxy-4-methoxyphenyl)piperidin-2-one;
(S)-5-(3-isopropoxy-4-methoxyphenyl)piperidin-2-one;
(S)-1-benzyl-5-(3-(cyclopentyloxy)-4-methoxyphenyl)piperidin-2-one;
(S)-5-(3-(cyclopentyloxy)-4-hydroxyphenyl)piperidin-2-one;
(S)-5-(3-(cyclopentyloxy)-4-propoxyphenyl)piperidin-2-one;
(S)-5-(3-(cyclopentyloxy)-4-(2-hydroxyethoxy)phenyl)piperidin-2-one;
(S)-5-(3-(cyclopentyloxy)-4-(3-hydroxypropoxy)phenyl)piperidin-2-one;
(S)-ethyl 4-(2-(cyclopentyloxy)-4-(6-oxopiperidin-3-yl)phenoxy)butanoate;
(S)-4-(2-(cyclopentyloxy)-4-(6-oxopiperidin-3-yl)phenoxy)butanoic acid;
(S)-1-acetyl-5-(3-(cyclopentyloxy)-4-methoxyphenyl)piperidin-2-one; and
(S)-1-benzoyl-5-(3-(cyclopentyloxy)-4-methoxyphenyl)piperidin-2-one.

Particularly preferred compounds of the invention, for example when R¹ represents optionally substituted C₁₋₁₂ alkyl, C₂₋₁₂ alkenyl or C₂₋₁₂ alkynyl, include:
(5S)-3-allyl-5-(3-(cyclopentyloxy)-4-methoxyphenyl)piperidin-2-one;
(5S)-5-(3-(cyclopentyloxy)-4-methoxyphenyl)-3-methylpiperidin-2-one; and
(S)-3,3-diallyl-5-(3-(cyclopentyloxy)-4-methoxyphenyl)piperidin-2-one.

Particularly preferred compounds of the invention, for example when R¹ represents -A^{1a}-N(R⁹)R¹⁰, include:
(5S)-5-(3-(cyclopentyloxy)-4-methoxyphenyl)-3-(2-(phenylamino)ethyl)piperidin-2-one;
(3R,5S)-5-(3-(cyclopentyloxy)-4-methoxyphenyl)-3-(2-(phenylamino)ethyl)piperidin-2-one;
4-(2-((5S)-5-(3-(cyclopentyloxy)-4-methoxyphenyl)-2-oxopiperidin-3-yl)ethylamino)benzonitrile;
(5S)-3-(2-(4H-1,2,4-triazol-4-ylamino)ethyl)-5-(3-(cyclopentyloxy)-4-methoxyphenyl)piperidin-2-one;
(3S,5S)-5-(3-(cyclopentyloxy)-4-methoxyphenyl)-3-(2-(4-fluorophenylamino)ethyl)piperidin-2-one;
(3R,5S)-5-(3-(cyclopentyloxy)-4-methoxyphenyl)-3-(2-(4-fluorophenylamino)ethyl)piperidin-2-one;
(3S,5S)-5-(3-(cyclopentyloxy)-4-methoxyphenyl)-3-((phenylamino)methyl)piperidin-2-one, MW 394.51;
(3S,5S)-5-(3-(cyclopentyloxy)-4-methoxyphenyl)-3-((4-isopropylphenylamino)methyl)piperidin-2-one, MW 436.59;
(3S,5S)-3-((4-chlorophenylamino)methyl)-5-(3-(cyclopentyloxy)-4-methoxyphenyl)piperidin-2-one;
(3S,5S)-5-(3-(cyclopentyloxy)-4-methoxyphenyl)-3-((m-tolylamino)methyl)piperidin-2-one;
(3S,5S)-5-(3-(cyclopentyloxy)-4-methoxyphenyl)-3-((4-fluorophenylamino)methyl)piperidin-2-one;
(3S,5S)-5-(3-(cyclopentyloxy)-4-methoxyphenyl)-3-((3-fluorophenylamino)methyl)piperidin-2-one;
(3S,5S)-5-(3-(cyclopentyloxy)-4-methoxyphenyl)-3-((3,5-dichlorophenylamino)methyl)piperidin-2-one;
(3S,5S)-5-(3-(cyclopentyloxy)-4-methoxyphenyl)-3-((3,4-dimethoxyphenylamino)methyl)piperidin-2-one;
(3S,5S)-5-(3-(cyclopentyloxy)-4-methoxyphenyl)-3-((3-phenoxyphenylamino)methyl)piperidin-2-one;
(3S,5S)-5-(3-(cyclopentyloxy)-4-methoxyphenyl)-3-((3,5-dimethoxyphenylamino)methyl)piperidin-2-one;
(3S,5S)-5-(3-(cyclopentyloxy)-4-methoxyphenyl)-3-((4-methoxyphenylamino)methyl)piperidin-2-one;
(3S,5S)-3-((benzo[d][1,3]dioxol-5-ylamino)methyl)-5-(3-(cyclopentyloxy)-4-methoxyphenyl)piperidin-2-one;
(3S,5S)-3-((4-tert-butylphenylamino)methyl)-5-(3-(cyclopentyloxy)-4-methoxyphenyl)piperidin-2-one;
(3S,5S)-5-(3-(cyclopentyloxy)-4-methoxyphenyl)-3-((3,4-difluorophenylamino)methyl)piperidin-2-one;
(3S,5S)-5-(3-(cyclopentyloxy)-4-methoxyphenyl)-3-((pyridin-4-ylamino)methyl)piperidin-2-one;
(3S,5S)-5-(3-(cyclopentyloxy)-4-methoxyphenyl)-3-((p-tolylamino)methyl)piperidin-2-one;
(3S,5S)-5-(3-(cyclopentyloxy)-4-methoxyphenyl)-3-((o-tolylamino)methyl)piperidin-2-one;
(3S,5S)-5-(3-(cyclopentyloxy)-4-methoxyphenyl)-3-((2-fluorophenylamino)methyl)piperidin-2-one;
(3S,5S)-5-(3-(cyclopentyloxy)-4-methoxyphenyl)-3-((2,6-dimethylphenylamino)methyl)piperidin-2-one;
(3S,5S)-5-(3-(cyclopentyloxy)-4-methoxyphenyl)-3-((3-ethoxyphenylamino)methyl)piperidin-2-one;
(3S,5S)-5-(3-(cyclopentyloxy)-4-methoxyphenyl)-3-((4-ethoxyphenylamino)methyl)piperidin-2-one;
(3S,5S)-5-(3-(cyclopentyloxy)-4-methoxyphenyl)-3-((3-nitrophenylamino)methyl)piperidin-2-one; and
(3S,5S)-3-((benzyl(phenyl)amino)methyl)-5-(3-(cyclopentyloxy)-4-methoxyphenyl)piperidin-2-one.

Particularly preferred compounds of the invention, for example when R¹ represents -A^{1c}-C(O)R⁹, include those in which:
(5S)-5-(3-(cyclopentyloxy)-4-methoxyphenyl)-3-(2-oxo-2-(4-(pyrrolidin-1-yl)piperidin-1-yl)ethyl)piperidin-2-one; and
2-((5S)-5-(3-(cyclopentyloxy)-4-methoxyphenyl)-2-oxopiperidin-3-yl)acetaldehyde.

Particularly preferred compounds of the invention, for example when R¹ represents -A^{1d}-C(O)OR⁹ or -A^{1e}-C(O)N(R⁹)R¹⁰, include those in which:
2-((5S)-5-(3-(cyclopentyloxy)-4-methoxyphenyl)-2-oxopiperidin-3-yl)acetic acid;
2-((5S)-5-(3-(cyclopentyloxy)-4-methoxyphenyl)-2-oxopiperidin-3-yl)-N-(1H-imidazol-2-yl)acetamide;
2-((3R,5S)-5-(3-(cyclopentyloxy)-4-methoxyphenyl)-2-oxopiperidin-3-yl)-N-(1H-imidazol-2-yl)acetamide;
2-((3S,5S)-5-(3-(cyclopentyloxy)-4-methoxyphenyl)-2-oxopiperidin-3-yl)-N-(1H-imidazol-2-yl)acetamide;
2-((5S)-5-(3-(cyclopentyloxy)-4-methoxyphenyl)-2-oxopiperidin-3-yl)-N-(pyridin-3-yl)acetamide;
2-((5S)-5-(3-(cyclopentyloxy)-4-methoxyphenyl)-2-oxopiperidin-3-yl)-N-(4-fluorophenyl)acetamide;
2-((5S)-5-(3-(cyclopentyloxy)-4-methoxyphenyl)-2-oxopiperidin-3-yl)-N-phenylacetamide;
N-(4-(1H-imidazol-1-yl)phenyl)-2-((5S)-5-(3-(cyclopentyloxy)-4-methoxyphenyl)-2-oxopiperidin-3-yl)acetamide;
2-((5R)-5-(3-(cyclopentyloxy)-4-methoxyphenyl)-2-oxopiperidin-3-yl)-N-phenylacetamide;
2-((5S)-5-(3-(cyclopentyloxy)-4-methoxyphenyl)-2-oxopiperidin-3-yl)-N-(quinolin-6-yl)acetamide; and
2-((5S)-5-(3-(cyclopentyloxy)-4-methoxyphenyl)-2-oxopiperidin-3-yl)-N-(pyridin-4-yl)acetamide.

Particularly preferred compounds of the invention, for example when R¹ represents -A¹-T^{z}-B¹, in which A¹ represents C₁₋₁₂ alkylene; r represents a direct bond; and B¹ represents heterocyclyl, include (3R,5S)-5-(3-(cyclopentyloxy)-4-methoxyphenyl)-3-((tetrahydrofuran-3-yl)methyl)piperidin-2-one.

Particularly preferred compounds of the invention, for example when R¹ and R² together represent ═C(R⁹)R¹⁰ include:
(S)-5-(3-(cyclopentyloxy)-4-methoxyphenyl)-3-methylenepiperidin-2-one;
(S,E)-3-benzylidene-5-(3-(cyclopentyloxy)-4-methoxyphenyl)piperidin-2-one;
(S,E)-5-(3-(cyclopentyloxy)-4-methoxyphenyl)-3-(4-fluorobenzylidene)piperidin-2-one;
(S,E)-5-(3-(cyclopentyloxy)-4-methoxyphenyl)-3-(2,4-difluorobenzylidene)piperidin-2-one;

(S,E)-5-(3-(cyclopentyloxy)-4-methoxyphenyl)-3-(3-fluorobenzylidene)piperidin-2-one;
(S,E)-5-(3-(cyclopentyloxy)-4-methoxyphenyl)-3-(3-phenoxybenzylidene)piperidin-2-one;
(S,E)-5-(3-(cyclopentyloxy)-4-methoxyphenyl)-3-((6-methylpyridin-2-yl)methylene)piperidin-2-one;
(S,E)-3-(4-(1H-imidazol-1-yl)benzylidene)-5-(3-(cyclopentyloxy)-4-methoxyphenyl)piperidin-2-one;
(S,E)-5-(3-(cyclopentyloxy)-4-methoxyphenyl)-3-(3-methoxybenzylidene)piperidin-2-one;
(S,E)-5-(3-(cyclopentyloxy)-4-methoxyphenyl)-3-(3-(trifluoromethoxy)benzylidene)piperidin-2-one;
(S,E)-5-(3-(cyclopentyloxy)-4-methoxyphenyl)-3-(4-(diethylamino)benzylidene)piperidin-2-one;
(S,E)-5-(3-(cyclopentyloxy)-4-methoxyphenyl)-3-(2-methoxybenzylidene)piperidin-2-one;
(S,E)-5-(3-(cyclopentyloxy)-4-methoxyphenyl)-3-(4-(diethoxymethyl)benzylidene)piperidin-2-one;
(S,E)-5-(3-(cyclopentyloxy)-4-methoxyphenyl)-3-(4-(3-(dimethylamino)propoxy)benzylidene)piperidin-2-one;
(S,E)-5-(3-(cyclopentyloxy)-4-methoxyphenyl)-3-(3,4-dimethoxybenzylidene)piperidin-2-one;
(S,E)-5-(3-(cyclopentyloxy)-4-methoxyphenyl)-3-(2,5-difluorobenzylidene)piperidin-2-one;
(S,E)-5-(3-(cyclopentyloxy)-4-methoxyphenyl)-3-(2-methylbenzylidene)piperidin-2-one;
(S,E)-3-(2-chlorobenzylidene)-5-(3-(cyclopentyloxy)-4-methoxyphenyl)piperidin-2-one;
(S,E)-5-(3-(cyclopentyloxy)-4-methoxyphenyl)-3-(2-nitrobenzylidene)piperidin-2-one;
(S,E)-5-(3-(cyclopentyloxy)-4-methoxyphenyl)-3-(3-methylbenzylidene)piperidin-2-one;
(S,E)-5-(3-(cyclopentyloxy)-4-methoxyphenyl)-3-(3-(trifluoromethyl)benzylidene)piperidin-2-one;
(S,E)-3-(3-chlorobenzylidene)-5-(3-(cyclopentyloxy)-4-methoxyphenyl)piperidin-2-one;
(S,E)-3-(benzo[d][1,3]dioxol-4-ylmethylene)-5-(3-(cyclopentyloxy)-4-methoxyphenyl)piperidin-2-one;
(S,E)-3-(benzo[d][1,3]dioxol-5-ylmethylene)-5-(3-(cyclopentyloxy)-4-methoxyphenyl)piperidin-2-one;
(S,E)-3-(2-chloro-5-(trifluoromethyl)benzylidene)-5-(3-(cyclopentyloxy)-4-methoxyphenyl)piperidin-2-one;
(S,E)-5-(3-(cyclopentyloxy)-4-methoxyphenyl)-3-(3,5-dichlorobenzylidene)piperidin-2-one;
(S,E)-5-(3-(cyclopentyloxy)-4-methoxyphenyl)-3-(furan-3-ylmethylene)piperidin-2-one;
(S,E)-5-(3-(cyclopentyloxy)-4-methoxyphenyl)-3-(pyridin-2-ylmethylene)piperidin-2-one;
(S,E)-5-(3-(cyclopentyloxy)-4-methoxyphenyl)-3-(pyridin-3-ylmethylene)piperidin-2-one;
(S,E)-5-(3-(cyclopentyloxy)-4-methoxyphenyl)-3-(pyridin-4-ylmethylene)piperidin-2-one;
(S,E)-3-(3-(4-chlorophenoxy)benzylidene)-5-(3-(cyclopentyloxy)-4-methoxyphenyl)piperidin-2-one;
(S,E)-5-(3-(cyclopentyloxy)-4-methoxyphenyl)-3-(2,3-difluorobenzylidene)piperidin-2-one;
(S,E)-3-(4-chlorobenzylidene)-5-(3-(cyclopentyloxy)-4-methoxyphenyl)piperidin-2-one;
(S,E)-5-(3-(cyclopentyloxy)-4-methoxyphenyl)-3-(4-fluoro-2-(trifluoromethyl)benzylidene)piperidin-2-one;
(S,E)-5-(3-(cyclopentyloxy)-4-methoxyphenyl)-3-(3-fluoro-4-methoxybenzylidene)piperidin-2-one;
(S,E)-3-(3,5-bis(trifluoromethyl)benzylidene)-5-(3-(cyclopentyloxy)-4-methoxyphenyl)piperidin-2-one;
(S,E)-5-(3-(cyclopentyloxy)-4-methoxyphenyl)-3-(3,4,5-trimethoxybenzylidene)piperidin-2-one;
(S,E)-5-(3-(cyclopentyloxy)-4-methoxyphenyl)-3-(4-phenoxybenzylidene)piperidin-2-one;
(S,E)-5-(3-(cyclopentyloxy)-4-methoxyphenyl)-3-(2,6-dimethoxybenzylidene)piperidin-2-one;
(S,E)-5-(3-(cyclopentyloxy)-4-methoxyphenyl)-3-(2-fluorobenzylidene)piperidin-2-one;
(S,E)-5-(3-(cyclopentyloxy)-4-methoxyphenyl)-3-(4-(trifluoromethoxy)benzylidene)piperidin-2-one;
(S,E)-5-(3-(cyclopentyloxy)-4-methoxyphenyl)-3-(4-isopropylbenzylidene)-piperidin-2-one; and
(S,E)-3-(4-butoxybenzylidene)-5-(3-(cyclopentyloxy)-4-methoxyphenyl)piperidin-2-one.

Particularly preferred compounds of the invention, for example when R¹ represents C₁₋₁₂ hydroxyalkyl, include those in which:
(5S)-5-(3-(cyclopentyloxy)-4-methoxyphenyl)-3-(2,3-dihydroxypropyl)piperidin-2-one;
(S)-5-(3-(cyclopentyloxy)-4-methoxyphenyl)-3,3-bis(hydroxymethyl)piperidin-2-one;
(5S)-5-(3-(cyclopentyloxy)-4-methoxyphenyl)-3-(hydroxymethyl)piperidin-2-one; and
5-(3-(cyclopentyloxy)-4-methoxyphenyl)-3-(3-methylbenzoyl)pyridin-2(1H)-one.

Compounds of formula I may be prepared by:

(i) for compounds of formula I in which R¹ represents -A¹-T^{z}-B¹, and T^{z} preferably represents a direct bond, reaction of a compound of formula II,

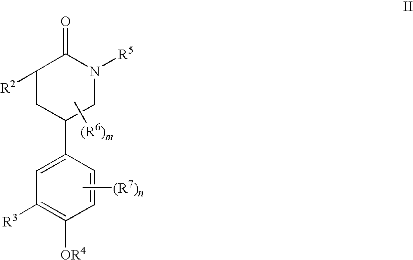

or protected derivatives thereof (e.g. a piperidin-2-one protected at the (1N)-position and/or the carbonyl protected as a silyl enol ether such as a tri-C₁₋₆ alkyl silyl enol ether), wherein R², R³, R⁴, R⁵, R⁶, R⁷, m and n are as hereinbefore defined, with a compound of formula III,

L¹-A¹-T^{z}-B¹ III

wherein L¹ represents a suitable leaving group, such as a sulfonate group or, more preferably an iodo, bromo or chloro group, T^{z} is as hereinbefore defined, but preferably represents a direct bond, and A¹ and B¹ are as hereinbefore defined, in the presence of a base, such as a strong base, for instance an organometallic (e.g. organolithium) base (such as n-BuLi, s-BuLi, t-BuLi, lithium 2,2,6,6-tetramethylpiperidine or, preferably, lithium diisopropylamide) or an alkali metalbased base such as NaH and/or KO-tert-butyl. When an organolithium base is employed, it is optionally in the presence of an additive (for example, a lithium coordinating agent such as an ether (e.g. dimethoxyethane) or an amine (e.g. tetramethylethylenediamine (TMEDA), (−)sparteine or 1,3-dimethyl-3,4,5,6-tetrahydro-2(1H)-pyrimidinone (DMPU) and the like)), for example in the presence of a suitable solvent, such as a polar aprotic solvent (e.g. tetrahydrofuran or diethyl ether), at sub-ambient temperatures (e.g. 0° C. to −78° C.) under an inert atmosphere. The skilled person will appreciate that the base may need to be added to the compound of formula II before the addition of the compound of formula III;
(ii) for compounds of formula I in which R¹ represents and T^{z} represents —N(R^{w1})—, or R¹ represents -A^{1a}-N(R⁹)R¹⁰ or -A^{1b}-OR⁹, reaction of a compound of formula IV,

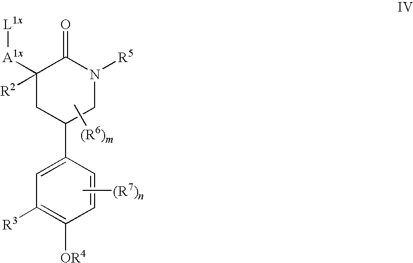

wherein L^{1x} represents a suitable leaving group, such as one hereinbefore defined in respect of L¹, A^{1x} represents A¹, A^{1a} or A^{1b} (as appropriate, i.e. for the preparation of compounds of formula I in which R¹ represents -A¹-N(R^{w1})-B¹, -A^{1a}-N(R⁹)R¹⁰ or -A^{1b}-OR⁹, respectively), and R², R³, R⁴, R⁵, R⁶, R⁷, m and n are as hereinbefore defined, with a compound of formula V,

H—Z^{a} V

wherein Z^{a} represents —N(R^{w1})—B¹, —N(R⁹)R¹⁰ or —OR⁹ (for the preparation of compounds of formula I in which R¹ represents -A¹-N(R^{w1})—B¹, -A^{1a}-N(R⁹)R¹⁰ or -A^{1b}-OR⁹, respectively), and R^{w1}, B¹, R⁹ and R¹⁰ are as hereinbefore defined, for example at around room temperature or above (e.g. up to 40-180° C.), optionally in the presence of a suitable base (e.g. sodium hydride, sodium bicarbonate, potassium carbonate, pyrrolidinopyridine, pyridine, triethylamine, tributylamine, trimethylamine, dimethylaminopyridine, diisopropylamine, diisopropylethylamine, 1,8-diazabicyclo[5.4.0]undec-7-ene, sodium hydroxide, N-ethyldiisopropylamine, N-(methylpolystyrene)-4-(methylamino)pyridine, potassium bis(trimethylsilyl)amide, sodium bis(trimethylsilyl)amide, potassium tert-butoxide, lithium diisopropylamide, lithium 2,2,6,6-tetramethylpiperidine or mixtures thereof) and an appropriate solvent (e.g. tetrahydrofuran, pyridine, toluene, dichloromethane, chloroform, acetonitrile, dimethylformamide, trifluoromethylbenzene, dioxane or triethylamine);
(iii) for compounds of formula I in which R¹ represents -A′-T^{Z}-B¹ and T^{z} represents —C(O)—N(R^{w2})—, or R¹ represents -A^{1e}-C(O)N(R⁹)R¹⁰, reaction of a compound of formula VI,

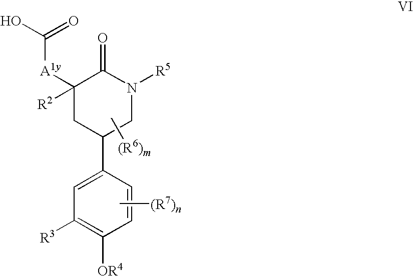

or a protected derivative thereof (e.g. an ester derivative), wherein A^{1y} represents A¹ or A^{1e} (as appropriate, i.e. for the preparation of compounds of formula I in which R¹ represents -A¹-C(O)—N(R^{w2})-B¹ or -A^{1e}-C(O)N(R⁹)R¹⁰, respectively), and R², R³, R⁴, R⁵, R⁶, R⁷, R⁹, R¹⁰, R^{w2}, B¹, A¹, A^{1e}, m and n are as hereinbefore defined, with a compound of formula VII,

H—Z^{b} VII

wherein Z^{b} represents —N(R^{w2})—B¹ or —N(R⁹)R¹⁰ (for the preparation of compounds of formula. I in which R¹ represents -A¹-C(O)—N(R^{w2})—B¹ or -A^{1e}-C(O)N(R⁹) respectively), and R^{w2}, B¹, R⁹ and R¹⁰ are as hereinbefore defined, under standard amide coupling reaction conditions, for example in the presence of a suitable coupling reagent (e.g. 1,1′-carbonyldiimidazole, N,N′-dicyclohexylcarbodiimide, 1-(3-dimethylaminopropyl)-3-ethylcarbodiimide (or hydrochloride thereof), N,N′-disuccinimidyl carbonate, benzotriazol-1-yloxytris(dimethylamino)phosphonium hexafluorophosphate, 2-(1H-benzotriazol-1-yl)-1,1,3,3-tetramethyluronium hexafluorophosphate, benzotriazol-1-yloxytrispyrrolidinophosphonium hexafluorophosphate, bromo-tris-pyrrolidinophosphonium hexafluorophosphate, 2-(1H-benzotriazol-1-yl)-1,1,3,3-tetramethyluronium tetrafluorocarbonate, 1-cyclohexyl-carbodiimide-3-propyloxymethyl polystyrene, O-(7-azabenzotriazol-1-yl)-N,N,N″,N″-tetramethyluronium hexafluorophosphate and/or O-benzotriazol-1-yl-N,N,N′,N=tetramethyluronium tetrafluoroborate), optionally in the presence of a suitable base (e.g. sodium hydride, sodium bicarbonate, potassium carbonate, pyridine, triethylamine, dimethylaminopyridine, diisopropylamine, sodium hydroxide, potassium tert-butoxide and/or lithium diisopropylamide (or variants thereof), an appropriate solvent (e.g. tetrahydrofuran, pyridine, toluene, dichloromethane, chloroform, acetonitrile, dimethylformamide, trifluoromethylbenzene, dioxane or triethylamine) and a further additive (e.g. 1-hydroxybenzotriazole hydrate). Alternatively, the carboxylic acid group of the compound of formula VI may be converted under standard conditions to the corresponding acyl chloride (e.g. in the presence of SOCl₂ or oxalyl chloride), which acyl chloride is then reacted with a compound of formula VII, for example under similar conditions to those mentioned above;
(iv) for compounds of formula I in which R¹ represents C₁₋₁₂ alkyl, C₂₋₁₂ alkenyl, C₂₋₁₂ alkynyl (which latter three groups are optionally substituted as defined herein), -A^{1a}-N(R⁹)R¹⁰, -A^{1b}-OR⁹, -A^{1c}-C(O)R⁹, -A^{1d-}C(O)OR⁹ or -A^{1e}-C(O)N(R⁹)R¹⁰, and R⁹ and R¹⁰ do not represent hydrogen (or appropriate protected derivatives thereof, e.g. for those compounds in which R¹ represents -A^{1d}-C(O)OR⁹, the latter group may contain a protected carboxylic acid moiety, e.g. -A^{1d}-C(O)O-tert-butyl), reaction of a compound of formula II as hereinbefore defined, with a compound of formula VIIA,

L^{1b}-Z^{c} VIIA

wherein L^{1b} represents a suitable leaving group, such as one hereinbefore defined in respect of L¹, Z^{c} represents C₁₋₁₂ alkyl, C₂₋₁₂ alkenyl, C₂₋₁₂ alkynyl (which latter three groups are optionally substituted as defined herein), -A^{1a}-N(R⁹)R¹⁰, -A^{1b}-OR⁹, -A^{1c}-C(O)R⁹, -A^{1d}-C(O)PR⁹ or -A^{1e}-C(O)N(R⁹)R¹⁰, but in which R⁹ and R¹⁰ represent a group other than hydrogen, for example under conditions such as those hereinbefore described in respect of process step (i) above, i.e. in the presence of base, etc. This reaction step may be particularly applicable to compounds of formula I in which R¹ represents optionally substituted C₁₋₁₂ alkyl, C₂₋₁₂ alkenyl or C₂₋₁₂ alkynyl, or -A^{1c}-C(O)R⁹ or -A^{1d}-C(O)OR⁹;
(v) for compounds of formula I in which R¹ and R² together form ═C(R⁹)R¹⁰, dehydration of a compound of formula VIIB,

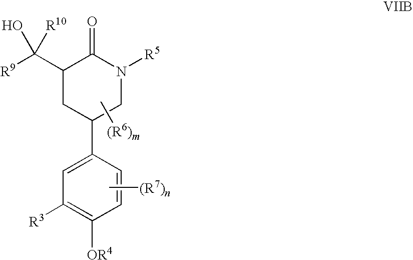

wherein R³, R⁴, R⁵, R⁶, R⁷, R⁹, R¹⁰, m and n are as hereinbefore defined, under standard dehydration reaction condition which involves the elimination of H₂O, for example under basic conditions or preferably under acidic conditions, such as reaction in the presence of a weakly acid (e.g. trifluoroacetic acid) in an aqueous solution;
(vi) intramolecular cyclisation of a compound of formula VIIC (in which R¹, R² and R⁵ all preferably represent hydrogen),

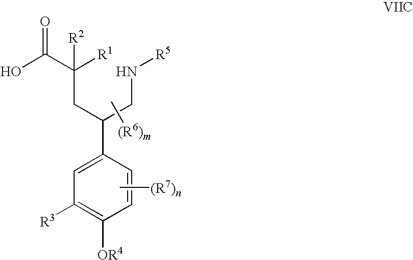

or a protected derivative thereof (e.g. an ester derivative such as a Cert-butyl ester thereof), wherein R¹, R², R³, R⁴, R⁵, R⁶, -R⁷, m and n are as hereinbefore defined, and R¹, R² and R⁵ preferably represent hydrogen (the skilled person will appreciate that —(R⁶)ₘ represents five optional R⁶ substituents situated at the five free positions a, (3 and y to the requisite —NH₂ group), under standard conditions, for example in the presence of a suitable base (such as an alkali metal base, e.g. NaOH, or an organic base, such as an amine base, e.g. triethylamine) an appropriate solvent (such as a polar solvent, e.g. dimethylformamide, or a solvent such as an alcohol, e.g. MeOH, which may have been employed in acid, e.g. para-toluene sulfonic acid, to promote a trans-esterificaiton or esterification of a compound of formula VIIC to form a methyl ester intermediate compound of formula VIIC). Such a process for the preparation of compounds of formula I, as well as alternative processes, are fully described in international patent application WO 2004/031149 and US patent U.S. Pat. No. 6,770,658, the disclosures of which are incorporated in full by reference herein. Alternatively, similar reaction conditions to those described hereinbefore with reference to process step (iii) may be employed;
(vii) compounds of formula I in which R¹ represents hydrogen and R² represents —OR⁴ in which R⁴ represents hydrogen may be prepared by reaction of a corresponding compound of formula I in which R² represents hydrogen, with a base (such as one described hereinbefore in respect of process step (i)), optionally in the presence of an additive and solvent (such as one hereinbefore described in respect of process step (i), for example Cu salts may be employed as the optional additive), followed by quenching with oxygen or a suitable equivalent thereof under standard conditions;
(viii) compounds of formula I in which R¹ represents hydrogen and R² represents —OR⁴ in which R⁴ represents hydrogen may be prepared by reaction of a compound of formula VIID,

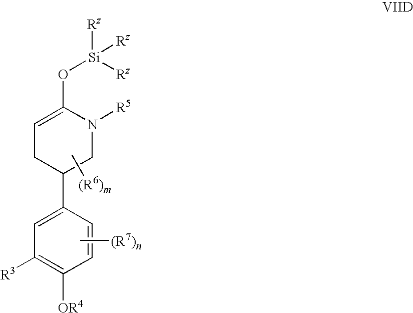

or a protected derivative thereof (e.g. protected at the (1N)-position), wherein each R^{z} independently represents C₁₋₆ alkyl (e.g. methyl, so forming for example a trimethylsilyl group), and R³, R⁴, R⁵, R⁶, R⁷, m and n are as hereinbefore defined, under double bond epoxidation reaction conditions known to those skilled in the art, for example in the presence of a suitable oxidising reagent such as meta chloro perbenzoic acid (mcpba). The skilled person will appreciate that an epoxide intermediate may be formed, which may not be stable and thus may hydrolyse during work-up to form the relevant compound of formula I (alternatively, the intermediate so formed may be deprotected e.g. under mild acidic conditions, or in the presence of fluoride ions, in order to promote the formation of the relevant compound of formula I);
(ix) compounds of formula I in which R¹ represents hydrogen and R² represents —OR⁴ in which R⁴ represents hydrogen may be prepared by reaction of a compound of formula VIIE,

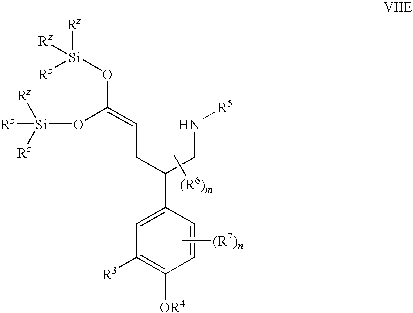

or a protected derivative thereof (e.g. protected at the (1N)-position), wherein R^{z}, R³, R⁴, R⁵, R⁶, R⁷, m and n are as hereinbefore defined, in the presence of a suitable oxidising agent, and under reaction conditions such as those hereinbefore described in respect of preparation of compounds of process step (viii) above;
(x) compounds of formula I may be prepared by reaction of a compound of formula VIII,

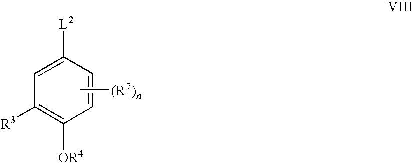

wherein L² represents a suitable leaving group such as chloro, bromo, iodo, a sulfonate group (e.g. —OS(O)₂CF₃, —OS(O)₂CH₃, —OS(O)₂PhMe or a nonaflate), —B(OH)₂, —B(OR^{wx})₂, —Sn(R^{wx})₃ or diazonium salts, in which each R^{wx} independently represents a C₁₋₆ alkyl group, or, in the case of —B(OR^{wx})₂, the respective groups may be linked together to form a 4- to 6-membered cyclic group (such as a 4,4,5,5-tetramethyl-1,3,2-dioxaborolan-2-yl group), and L² preferably represents bromo, and R³, R⁴, R⁷ and n are as hereinbefore defined, with a compound of formula IX,

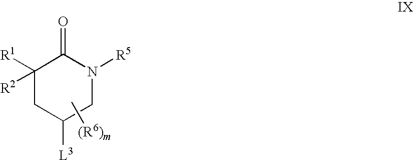

or a tautomer thereof or derivative thereof (including a possible derivative of the tautomer, e.g. a hydroxy protected tautomer, or a (1N)-protected derivative), wherein L³ represents a suitable leaving group, such as —B(OH)₂ or a protected derivative thereof, for example a 4,4,5,5-tetramethyl-1,3,2-dioxaborolan-2-yl group, 9-borabicyclo[3.3.1]-nonane (9-BBN), —Sn(alkyl)₃ (e.g. —SnMe₃ or —SnBu₃), or a similar group known to the skilled person and L³ preferably represents —B(OH)₂ (the skilled person will also appreciate that L² and L³ should be mutually compatible, and may also be interchanged), and R², R⁵, R⁶, L³ and m are as hereinbefore defined, for example in the presence of a suitable catalyst system, e.g. a metal (or a salt or complex thereof) such as CuI, Pd/C, PdCl₂, Pd(OAc)₂, Pd(Ph₃P)₂Cl₂, Pd(Ph₃P)₄, Pd₂(dba)₃ or NiCl₂ and a ligand such as t-Bu₃P, (C₆H₁₁)₃P, Ph₃P, AsPh₃, P(o-Tol)₃, 1,2-bis(diphenylphosphino)ethane, 2,2′-bis(ditert-butylphosphino)-1,1′-biphenyl, 2,2′-bis(diphenylphosphino)-1,1′-bi-naphthyl, 1,1′-bis(diphenylphosphinoferrocene), 1,3-bis(diphenyl-phosphino)propane, xantphos, or a mixture thereof, together with a suitable base such as, Na₂CO₃, K₃PO₄, Cs₂CO₃, NaOH, KOH, K₂CO₃, CsF, Et₃N, (i-Pr)₂NEt, t-BuONa or t-BuOK (or mixtures thereof) in a suitable solvent such as dioxane, toluene, ethanol, dimethylformamide, ethylene glycol dimethyl ether, water, dimethylsulfoxide, acetonitrile, dimethylacetamide, N-methylpyrrolidinone, tetrahydrofuran or mixtures thereof. The reaction may also be carried out for example at room temperature or above (e.g. at a high temperature such as the reflux temperature of the solvent system) or using microwave irradiation;
(xi) for compounds of formula I in which R² represents hydrogen, and there is a maximum of two R⁶ substituents present (at the 4- and/or 6-position), reduction of a compound of formula IXA,

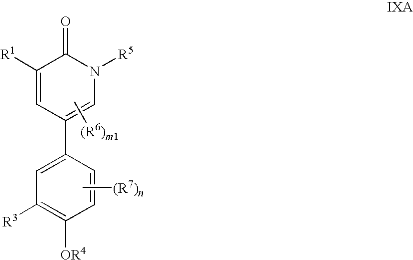

or a tautomer or protected derivative thereof (e.g. a protected hydroxy tautomer, or, a compound protected at the (1N)-position), wherein m1 represents 0, 1 or 2 (and a single R⁶ group is therefore possible at the 4- and/or 6-position of the piperidin-2-one ring), and R¹, R³, R⁴, R⁵, R⁶, R⁷ and n are as hereinbefore defined, for example under standard conditions, such as in the presence of a suitable reducing agent such as NaBH₄ (e.g. in the presence of a suitable additive), LiAlH₄ or under hydrogenation reaction conditions (e.g. catalytic hydrogenation conditions in the presence of a precious metal catalyst, e.g. Pd/C);
(xii) for compounds of formula I in which R³ represents —OR⁴ in which R⁴ is other than hydrogen, or for compounds of formula I in which R⁴ (at the position para to the point of attachment of the piperidin-2-one ring) is other than hydrogen, reaction of a corresponding compound of formula I in which R³ represents —OH or, R⁴ represents hydrogen, with a compound of formula IXB,

R^{4a}—L^{2x} IXB

wherein R^{4a} represents —R⁸—OR⁹, —R⁸—C(O)OR⁹, C₁₋₁₂ alkyl, C₂₋₁₂ alkenyl, C₂₋₁₂ alkynyl (which latter three groups are optionally substituted by one or more substituents selected from X³) or -A³-B³, L^{2x} represents a suitable leaving group such as one defined hereinbefore in respect of L², and R⁸, R⁹, X³, A³ and B³ are as hereinbefore defined, under standard reaction conditions, for example such as those hereinbefore described in respect of process step (ii) or (x). The skilled person will appreciate that when the reaction is with a compound of formula IXB in which L^{2x} is e.g. bromo, chloro or a sulfonate group, then the conditions described in process step (ii) are preferably employed, whereas for reaction with a compound of formula IXB in which L^{2x} is —B(OH)₂, —B(OR^{wx})₂ or —Sn(R^{wx})₃, then the reaction is preferably preformed under the reaction conditions described in process step (x);
(xiii) for compounds of formula I in which R³ represents —OR⁴ in which R⁴ is other than hydrogen, or for compounds of formula I in which R⁴ (at the position para to the point of attachment of the piperidin-2-one ring) is other than hydrogen, reaction of a compound of formula IXC,

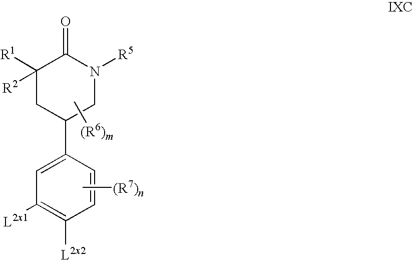

wherein L^{2x1} represents L^{2x} or R³, L^{2x2} represents L^{2x} or —OR⁴, provided that at least one of R^{2x1} and R^{2x2} represents L^{2x}, in which L^{2x} is as hereinbefore defined and preferably represents a suitable leaving group such as bromo, and R¹, R², R⁵, R⁶, R⁷, m and n are as hereinbefore defined, with a compound of formula IXD,

R⁴—OH IXD

wherein R⁴ is as hereinbefore defined, under standard reaction conditions, for example such as those hereinbefore described in respect of process (xii) (preferably with reference to process (x)).

Compounds of formula II in which m represents 0, 1 or 2 (forming a compound of formula II in which a R⁶ group is optionally present at the 4- and/or 6-position of the piperidin-2-one) may be prepared by reduction of a compound of formula IXE,

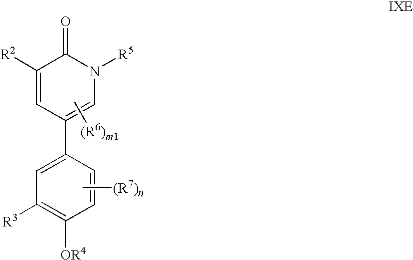

or a tautomer or protected derivative thereof (e.g. a protected hydroxy tautomer, or, a compound protected at the (1N)-position), wherein ml, R², R³, R⁴, R⁵, R⁶, R⁷ and n are as hereinbefore defined, for example under reaction conditions such as those hereinbefore described in respect of preparation of compounds of formula I (process step (xi) above).

Compound of formula III in which B¹ represents a fragment of formula X,

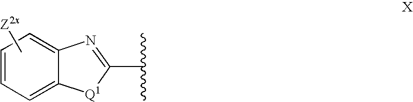

wherein Q¹ represents —O—, —S— or —N(H)—, and Z^{2x} represents one or more optional Z^{2a} substituents that may be present at any position on the 6,5-bicycle (including the —N(H)— position; and hence Q¹ may represent —N(R^{zz})—, in which R^{zz} represents e.g. C₁₋₁₂ alkyl, C₂₋₁₂ alkenyl or C₂₋₁₂ alkynyl, all of which are optionally substituted as hereinbefore defined), reaction of a compound of formula XI,

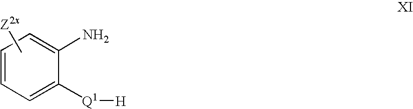

wherein Q¹ and Z^{2x} are as hereinbefore defined, with either a compound of formula XII,

L¹-A¹-C(═NH)OR^{t1} XII

or a salt thereof (e.g. a hydrochloride salt), wherein R^{t1} represents optionally substituted C₁₋₆ alkyl (e.g. ethyl) and L¹ and A¹ are as hereinbefore defined, or a compound of formula XIII,

L¹-A¹-C(O)OH XIII

or a protected derivative thereof (e.g. an ester derivative), wherein L¹ and A¹ are as hereinbefore defined, for example under standard cyclisation conditions known to those skilled in the art, such as in the presence of a suitable solvent (e.g. an aprotic solvent).

Alternatively, compounds of formula III in which L¹ represents bromo or a sulfonate group may be prepared by reaction of compounds of formula XIV,

HO-A¹-B¹ XIV

wherein A¹ and B¹ are as hereinbefore defined, under bromination reaction conditions (in the case of formation of compounds of formula III in which L¹ represents bromo) for example in the presence of a suitable brominating reagent (e.g. in the presence of PBr₃ or CBr₄, optionally in the presence of PPh₃, and optionally in the presence of a suitable solvent such as dichloromethane or diethyl ether), or by reaction in the presence of a sulfonyl chloride (in the case of formation of compounds of formula III in which L¹ represents a sulfonate group), for example in the presence of a suitable base and suitable solvent (for example a base such as one described hereinbefore in respect of preparation of compounds of formula II, in the presence of a solvent such as dichloromethane).

Compounds of formula VI may be prepared by reaction of a compound of formula II as hereinbefore defined, with a compound of formula XV,

L^{1y}-A¹-C(O)OH XV

or a protected derivative (e.g. ester) thereof, wherein L^{1y} represents a suitable leaving group, for example one hereinbefore defined in respect of L¹ (e.g. bromo) and A¹ is as hereinbefore defined, under standard reaction conditions known to those skilled in the art, for example such as those hereinbefore defined in respect of preparation of compounds of formula I (process step (i) above).

Compounds of formula VIIB may be prepared by reaction of a compound of formula II as hereinbefore defined but in which R² represents hydrogen, with a compound of formula XVA,

R⁹—C(O)—R¹⁰ XVA

wherein R⁹ and R¹⁰ are as hereinbefore defined, for example under reaction conditions such as those hereinbefore described in respect of preparation of compounds of formula I (process step (i)), i.e. in the presence of a suitable base, optional additive and solvent such as those described in that process step. The skilled person will appreciate that the base may need to be added to the reaction mixture before the addition of the compound of formula XVA.

Compounds of formula VIID may be prepared by reaction of a corresponding compound of formula II in which R² represents hydrogen (or a suitable protected derivative thereof, such as a (1N)-protected derivative), with an appropriate trialkylsilyl chloride (e.g. trimethylsilyl chloride), or the like, under standard reaction conditions, for example such as those hereinbefore described in respect of preparation of compounds of formula I (process step (ii) above).

Compounds of formula VIIE may be prepared from corresponding compounds of formula VIIC in which R¹ and R² both represent hydrogen, (or a suitable protected derivative thereof, such as a (1N)-protected derivative), with an appropriate trialkylsilyl chloride (e.g. trimethylsilyl chloride), or the like, under standard reaction conditions.

Compounds of formula IXA and IXE may be prepared by reaction of a compound of formula VIII as hereinbefore defined, with a compound of formula XVB,

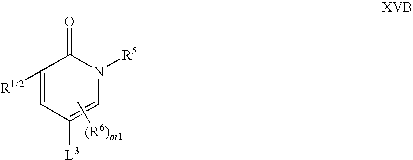

or a tautomer thereof or derivative thereof (including a possible derivative of the tautomer, e.g. a hydroxy protected tautomer, or, a protected derivative, such as a (1N)-nitrogen protected derivative, e.g. N-benzyl-2-piperidinone, or, 2-methoxypyridine or 2-chloropyridine), wherein R^{1/2} represents R¹ (for the preparation of compounds of formula IXA) or R² (for the preparation of compounds of formula IXE), and L³, m1, R¹, R², R⁵ and R⁶ are as hereinbefore defined, for example under conditions such as those hereinbefore described in respect of preparation of compounds of formula I (process step (x) above). Alternatively, the values of L² and L³ in the compounds of formula VIII and XVB may be interchanged.

Alternatively, compounds of formula IXA in which R¹ represents a substituent other than hydrogen, or compounds of formula IXE in which R² represents C₁₋₁₂ alkyl, C₂₋₁₂ alkenyl or C₂₋₁₂ alkynyl, all of which are optionally substituted as hereinbefore defined, may be prepared by reaction of a compound of formula XVC,

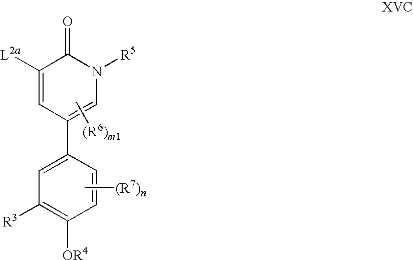

wherein L^{2a} represents a suitable leaving group, such one hereinbefore defined in respect of L², and m1, n, R³, R⁴, R⁵, R⁶ and R⁷ are as hereinbefore defined, with a compound of formula XVD,

R^{1/2a}-L^{3a} XVD

wherein L^{3a} represents a suitable leaving group, such as one hereinbefore defined in respect of L³ (or, alternatively, the definitions of L^{2a} and L^{3a} may be interchanged), R^{1/2a} represents R¹, provided that it does not represent hydrogen (for the preparation of the relevant compounds of formula IXA) or R², provided that it does not represent hydrogen or —OR⁴ (for the preparation of the relevant compounds of formula IXE), for example under conditions such as those hereinbefore described in respect of preparation of compounds of formula I (process step (x) above).

For compounds of formula IXE in which R² represents —OR⁴, reaction with a compound of formula XVC as hereinbefore defined, with a compound of formula IXD as hereinbefore defined, under standard reaction conditions, for example under conditions such as those hereinbefore described in respect of preparation of compounds of formula I (process step (x) above).

Alternatively, compounds of formula IXA or compounds of formula IXE in which m1 represents 1 or 2, may be prepared by reaction of a compound of formula XVE,

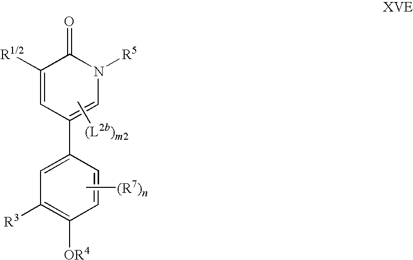

wherein L^{2b} represents a suitable leaving group, such as one hereinbefore defined in respect of L², m2 represents 1 or 2 (and hence there are one or two L^{2b} groups present at the 4- and/or 6-position of the 2-pyridinone ring), and R³, R⁴, R⁵, R⁷, R^{1/2} and n are as hereinbefore defined, with a compound of formula XVF,

R⁶-L^{3b} XVF

wherein L^{3b} represents a suitable leaving group, such as one hereinbefore defined in respect of L³ (or alternatively, the values of L^{2b} and L^{3b} may be interchanged), and R⁶ is as hereinbefore defined, under standard reaction conditions, for example under conditions such as those hereinbefore described in respect of preparation of compounds of formula I (process step (x) above).

Compounds of formula XII may be prepared by reaction of a compound of formula XVI,

L¹-A¹-CN XVI

wherein L¹ and A¹ are as hereinbefore defined, in the presence of a compound of formula XVII,

HO—R^{t1} XVII

wherein R^{t1} is as hereinbefore defined, and which compound of formula XVI may be present as solvent, in the presence of acetyl chloride and optionally in the presence of a suitable solvent such as chloroform.

Compounds of formula XIV may be prepared by reduction of a compound of formula XVIII,

A^{1x}-B¹ XVIII

wherein A^{1x} represents C₁₋₁₂ alkylene optionally substituted by one or more substituents selected from X⁷ and terminally substituted with a ═O group, under standard reduction conditions, for example in the presence of a suitable reducing reagent such as LiAlH₄, NaBH₄ or LiBH₄, in the presence of a suitable solvent (such as an alcoholic solvent, e.g. methanol or ethanol).

Compounds of formulae IV, V, VII, VIIA, VIIC, VIII, IX, X, XI, XIII, XV, XVA, XVB, XVC, XVD, XVE, XVF, XVI, XVII, XVIII (and also e.g. certain compounds of formulae II, III and VI) may be commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further, compounds of formula II may also be prepared in accordance with synthetic routes and techniques described in international patent applications WO 2004/031149 and WO 00/14083 and/or US patent U.S. Pat. No. 6,770,658.

The substituents L¹, R¹, R², R³, R⁴, R⁵, R⁶ and R⁷ either in final compounds of the invention or in relevant intermediates (as appropriate) may be modified one or more times, after or during the processes described above by way of methods that are well known to those skilled in the art. Examples of such methods include substitutions, reductions, oxidations, alkylations, acylations, hydrolyses, esterifications, etherifications, halogenations or nitrations. Such reactions may result in the formation of a symmetric or asymmetric final compound of the invention or intermediate. In this respect, the skilled person may also refer to “Comprehensive Organic Functional Group Transformations” by A. R. Katritzky, O. Meth-Cohn and C. W. Rees, Pergamon Press, 1995: Specific transformation steps that may be mentioned include the conversion of one L¹ group (in the compound of formula III) into another L¹ group (e.g. the conversion of one halo group, such as chloro, into another halo group, such as iodo, for example by reaction in the presence of potassium iodide), or even the conversion of a hydroxy group to a L¹ group. Other transformation steps include the reduction of a nitro group to an amino group, the hydrolysis of a nitrile group to a carboxylic acid group, and standard nucleophilic aromatic substitution reactions.

It will also be appreciated by those skilled in the art that in the process described below the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (e.g., t-butyldimethylsilyl, t-butyldiphenylsilyl or trimethylsilyl), tetrahydropyranyl, benzyl, methyl and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or aralkyl), p-methoxybenzyl, trityl and the like. Suitable protecting groups for carboxylic acid include alkyl, aryl or aralkyl esters. Further, a carbonyl group may be protected as the silyl enol ether, which may be introduced under standard conditions, and converted back to the enolate (or carbonyl compound) by reaction in the presence of fluoride ions (or a suitable source thereof).

Protecting groups may be added or removed in accordance with standard techniques (for example a methyl protecting group on a hydroxy group may be removed by reaction in the presence of a suitable ‘cleaving reagent’ such as BBr₃), which are known to one skilled in the art and as described herein. The use of protecting groups is described in detail in Green, T. W. and P. G. M. Wuts, Protective Groups in Organic Synthesis (1999), 3rd Ed., Wiley.

The protecting group may also be a polymer resin such as a Wang resin or a 2-chlorotrityl-chloride resin.

It will also be appreciated by those skilled in the art, although such protected derivatives of compounds of this invention may not possess pharmacological activity as such, they may be administered to a mammal and thereafter metabolised in the body to form compounds of the invention which are pharmacologically active. Such derivatives may therefore be described as “prodrugs”. All prodrugs of compounds of this invention are included within the scope of the invention.

### Medical and Pharmaceutical Uses and Testing of the Compounds of the Invention

Compounds of the invention are indicated as pharmaceuticals. According to a further aspect of the invention there is provided a compound of the invention, as hereinbefore defined but without provisos (C) and (D), for use as a pharmaceutical. Alternatively, there is provided a compound of the invention, as hereinbefore defined in conjunction with proviso (X^{a}), for use as a pharmaceutical.

According to a further aspect of the invention there is provided a pharmaceutical composition/formulation including a compound of the invention as hereinbefore defined, but without provisos (C) and (D) (or a compound of the invention, as hereinbefore defined in conjunction with proviso (X^{a})), in admixture with a pharmaceutically acceptable adjuvant, carrier, diluent or excipient.

Preferred pharmaceutical formulations include those in which the active ingredient is present in at least 1% (such as at least 10%, preferably in at least 30% and most preferably in at least 50%) by weight. That is, the ratio of active ingredient to the other components (i.e. the addition of adjuvant, diluent and carrier) of the pharmaceutical composition is at least 1:99 (e.g. at least 10:90, preferably at least 30:70 and most preferably at least 50:50) by weight.

Such compositions/formulations may be prepared in accordance with standard and/or accepted pharmaceutical practice.

Compounds of the invention may be useful in treating or preventing inflammatory diseases or conditions in a patient. Hence, in another aspect, this invention is directed to methods for treating or preventing an inflammatory disease or condition in a mammal, preferably a human, wherein the method comprises administering to the mammal in need thereof a therapeutically effective amount of a compound of the invention as hereinbefore described but without provisos (B), (C) and (D) (or a compound of the invention, as hereinbefore defined in conjunction with proviso (X^{a}) but without proviso (B)) or a therapeutically effective amount of a pharmaceutical formulation/composition of the invention as hereinbefore described but preferably also without proviso (B).

The term “inflammation” will thus also be understood to include any inflammatory disease, disorder or condition per se, any condition that has an inflammatory component associated with it, and/or any condition characterised by inflammation as a symptom. Accordingly, compounds of the invention (e.g. those hereinbefore defined, but without provisos (B), (C) and (D), or, compounds of the invention, as hereinbefore defined in conjunction with proviso (X^{a}) but without proviso (B)) may be useful in the treatment of the inflammatory diseases or conditions described herein, and/or (if appropriate) inflammation that may be associated with such diseases or conditions.

The inflammatory condition or disease may be an autoimmune condition or disease; the inflammatory condition or disease may involve acute or chronic inflammation of bone and/or cartilage compartments of joints; the inflammatory condition or disease may be an arthritis selected from rheumatoid arthritis, gouty arthritis or juvenile rheumatoid arthritis; the inflammatory condition or disease may be a respiratory disorder selected from asthma or a chronic obstructive pulmonary disease (COPD, e.g., emphysema or chronic bronchitis); the condition or disease may be associated with the disregulation of T-cells; the condition or disease may be associated with elevated levels of inflammatory cytokines (e.g., wherein the inflammatory cytokine is IL-2, or wherein the inflammatory cytokine is IFN-γ, or wherein the inflammatory cytokine is TNF-α); the inflammatory condition or disease may be multiple sclerosis; the inflammatory condition or disease may be pulmonary sarcadosis; the inflammatory condition or disease may be ocular inflammation or allergy; the inflammatory condition or disease may be an inflammatory bowel disease (e.g., Crohn's disease or ulcerative colitis); and the inflammatory condition or disease may be an inflammatory cutaneous disease (e.g., psoriasis or dermatitis). Compounds of the invention (e.g. those hereinbefore defined, but without provisos (B), (C) and (D), or, compounds of the invention, as hereinbefore defined in conjunction with proviso (X^{a}) but without proviso (B)) may be useful in modulating intracellular cyclic adenosine 5′-monophosphate levels within a mammal, preferably a human, Hence, in another aspect, this invention is directed to methods for modulating intracellular cyclic adenosine 5′-monophosphate levels within a mammal, preferably a human, wherein the method comprises administering to the mammal in need thereof an amount of a compound of the invention (e.g. those hereinbefore defined, but without provisos (B), (C) and (D) (or compounds of the invention, as hereinbefore defined in conjunction with proviso (X^{a}) but without proviso (B)) or a pharmaceutical formulation/composition of the invention as hereinbefore described (but preferably also without proviso (B)) effective to modulate the intracellular cyclic adenosine 5′-monophosphate levels of the mammal. The mammal, preferably a human, may have an inflammatory condition or disease (for example one defined herein).

Compounds of the invention (e.g. those hereinbefore defined, but without provisos (B), (C) and (D), or, compounds of the invention, as hereinbefore defined in conjunction with proviso (X^{a}) but without proviso (B)) may be useful in treating or preventing a disease or condition in a mammal, preferably a human, where the disease or condition is associated with pathological conditions that are modulated by inhibiting enzymes associated with secondary cellular messengers. Hence, in another aspect, this invention is directed to methods for treating or preventing a disease or condition in a mammal, preferably a human, wherein the method comprises administering to the mammal in need thereof a therapeutically effective amount of a compound of the invention (e.g. those hereinbefore defined, but without provisos (B), (C) and (D) (or compounds of the invention, as hereinbefore defined in conjunction with proviso (X^{a}) but without proviso (B)) or a pharmaceutical formulation/composition of the invention as hereinbefore described (but preferably also without proviso (B)), and the disease or condition is associated with pathological conditions that are modulated by inhibiting enzymes associated with secondary cellular messengers. Such enzymes (that may be inhibited) may be a cyclic AMP phosphodiesterase; a phosphodiesterase 4; a phosphodiesterase 3; or a cyclic GMP phosphodiesterase. Further, more than one type of enzyme may be inhibited, for instance, the enzymes may be both phosphodiesterase 4 and phosphodiesterase 3.

Compounds of the invention (e.g. those hereinbefore defined, but without provisos (B), (C) and (D), or, compounds of the invention, as hereinbefore defined in conjunction with proviso (X^{a}) but without proviso (B)) may be useful in treating or preventing uncontrolled cellular proliferation in a mammal, preferably a human. Hence, in another aspect, this invention is directed to methods for treating or preventing uncontrolled cellular proliferation in a mammal, preferably a human, wherein the method comprises administering to the mammal in need thereof a therapeutically effective amount (e.g. an amount effective to treat or prevent uncontrolled cellular) of a compound of the invention (e.g. those hereinbefore defined, but without provisos (B), (C) and (D), or, compounds of the invention, as hereinbefore defined in conjunction with proviso (X^{a}) but without proviso (B)) or a pharmaceutical formulation/composition of the invention as hereinbefore described (but preferably also without proviso (B)). The uncontrolled cellular proliferation may be caused by a cancer selected from leukaemia and solid tumors.

Compounds of the invention (e.g. those hereinbefore defined, but without provisos (B), (C) and (D), or, compounds of the invention, as hereinbefore defined in conjunction with proviso (X^{a}) but without proviso (B)) may be useful in treating or preventing transplant rejection in a mammal, preferably a human. Hence, in another aspect, this invention is directed to methods for treating or preventing transplant rejection in a mammal, preferably a human, wherein the method comprises administering to the mammal in need thereof a therapeutically effective amount (e.g. an amount effective to treat or prevent transplant rejection in the mammal) of a compound of the invention (e.g. those hereinbefore defined, but without provisos (B), (C) and (D), or, compounds of the invention, as hereinbefore defined in conjunction with proviso (X^{a}) but without proviso (B)) or a pharmaceutical formulation/composition of the invention as hereinbefore described (but preferably also without proviso (B)). The rejection may be due to graft versus host disease.

Compounds of the invention (e.g. those hereinbefore defined, but without provisos (B), (C) and (D), or, compounds of the invention, as hereinbefore defined in conjunction with proviso (X^{a}) but without proviso (B)) may be useful in treating or preventing conditions associated with the central nervous system (CNS) in a mammal, preferably a human. Hence, in another aspect, this invention is directed to methods for treating or preventing conditions associated with the central nervous system in a mammal, preferably a human, wherein the method comprises administering to the mammal in need thereof a therapeutically effective amount (e.g. an amount effective to treat or prevent conditions associated with the central nervous system (CNS) in the mammal) of a compound of the invention as described above (e.g. those hereinbefore defined, but without provisos (B), (C) and (D), or, compounds of the invention, as hereinbefore defined in conjunction with proviso (X^{a}) but without proviso (B)) or a pharmaceutical formulation/composition of the invention as hereinbefore described (but preferably also without proviso (B)). The condition associated with the central nervous system (CNS) may be depression.

These and other aspects and embodiments of the present invention will be apparent upon reference to the following detailed description. To this end, various references are set forth herein which describe in more detail certain procedures, compounds and/or formulations/compositions, and are hereby incorporated by reference in their entirety.

In a method of the present invention, a compound of the invention (as hereinbefore defined, e.g. compounds of the invention as hereinbefore defined, but without provisos (B), (C) and (D), or, compounds of the invention, as hereinbefore defined in conjunction with proviso (X^{a}) but without proviso (B)), or a formulation/composition comprising one or more compounds of the invention (as hereinbefore defined, but preferably also without proviso (B)) in admixture with a pharmaceutically acceptable adjuvant, carrier, diluent or excipient, may, although need not, achieve one or more of the following desired results in a subject to whom has been administered a compound of the invention as hereinbefore defined, or a formulation/composition containing one of these compounds and a pharmaceutically acceptable adjuvant, carrier, diluent or excipient:

1. Inhibition of reactive oxygen species generation from primary neutrophils;
2. Inhibition of neutrophil chemotaxis;
3. Inhibition of TNF-α production;
4. Inhibition of edema;
5. Oxygen radical scavenging;
6. Inhibition of cyclic-AMP phosphodiesterases 1, 3 and/or 4, and related PDEs such as PDE7;
7. Potentiate induction of CRE-mediated transcription activity in human monocytic cells;
8. Inhibition of PDE, preferably PDE4, PDE3, or PDE3 and PDE4;
9. Inhibition of cytokine production by activated T-cell subsets;
10. Inhibition of neutrophil myeloperoxidase release;
11. Low ratio of IC50 PDE4(cat):IC50PDE4(HARBS);
12. Inhibition of graft rejection;
13. Inhibition of clinical and histopathological parameters of disease in inflammatory bowel disease;
14. Inhibition of clinical and histopathological parameters of arthritis in a murine collage-induced arthritis model;
15. Inhibition of clinical and histopathological parameters of disease in asthma; and
16. Inhibition of clinical and histopathological parameters of disease in COPD.

Thus, the compounds and compositions of the invention (as defined herein) may be used to treat inflammation, including both acute and chronic inflammation as well as certain proliferative disorders (cancers). As used herein, inflammation includes, without limitation, ankylosing spondylitis, arthritis (where this term encompasses over 100 kinds of rheumatic diseases), asthma, chronic bronchitis, Crohn's disease, fibromyalgia syndrome, gout, inflammations of the brain (including multiple sclerosis, AIDS dementia, Lyme encephalopathy, herpes encephalitis, Creutzfeld-Jakob disease, and cerebral toxoplasmosis), emphysema, inflammatory bowel disease, irritable bowel syndrome, ischemia-reperfusion injury juvenile erythematosus pulmonary sarcoidosis, Kawasaki disease, osteoarthritis, pelvic inflammatory disease, psoriatic arthritis (psoriasis), rheumatoid arthritis, psoriasis, tissue/organ transplant, scleroderma, spondyloarthropathies, systemic lupus erythematosus, pulmonary sarcoidosis, and ulcerative colitis. As used herein, proliferative disorders includes, without limitation, all leukemias and solid tumors that are susceptible to undergoing differentiation or apoptosis upon interruption of their cell cycle.

The compounds of the invention (as defined herein) may be tested for the above indications in the assays described below in the Biological Examples. In addition, the compounds of the invention may be tested in animal models to further demonstrate their enzymatic, cellular, anti-inflammatory and central nervous system activity. Specifically, the compounds of the invention may be tested in animal models for diseases and pathological conditions of the central nervous system, including, but not limited to, cognitive function, Alzheimer's disease, learning and memory (Rose et al., “Phosphodiesterase inhibitors for cognitive enhancement,” Curr. Pharm. Des. 2005; 11(26):3329-34), Rubinstein Taybi Syndrome (Bourtchouladze et al., “A mouse model of Rubinstein-Taybi syndrome: Defective long term memory is amerliorated by inhibitors of phosphodiesterase 4, Proc. Natl. Acad. Sci. USA. (2003), September 2;100(18):10518-22.), cerebrovascular disease, depression (Zhu et al., “The antidepressant and antiinflammatory effects of rolipram in the central nervous system”, CNS Drug Rev. (2001);7(4):387-98), schizophrenia, Parkinson's disease (Weishaar et al., “A new generation of phosphodiesterase inhibitors: multiple molecular forms of phosphodiesterase and the potential for drug selectivity”, J Med. Chem. (1985), May; 28(5):537-45.), multiple sclerosis (Huang et al., “The next generation of PDE4 inhibitors”, Curr Opin Chem. Biol. (2001), August; 5(4):432-8; Dyke and Montana, “Update on the therapeutic potential of PDE4 inhibitors”, Expert Opin Investig Drugs. (2002), January; 11(1):1-13) and allergic rhinitis. In addition, the compounds of the invention may be tested in animal models for inflammatory and immune disorders or pathological conditions including, but not limited to, cancer (Weishaar et al., 1985), asthma (Huang et al., 2001; Dyke and Montana, 2002), chronic obstructive pulmonary disease (Huang et al., 2001; Dyke and Montana, 2002), respiratory distress syndrome, rhinitis, nephritis, psoriasis (Houslay et al., “Keynote review: phosphodiesterase-4 as a therapeutic target”, Drug Discov Today. (2005), November 15; 10(22):1503-19), eczema, atopic dermatitis, urticaria, conjunctivitis, inflammatory bowel diseases (Huang et al., 2001), Crohn's disease, ulcerative colitis, rheumatoid arthritis (Huang et al., 2001), osteoarthritis, eosinophilic gastrointestinal disorders, vascular disease and diabetes mellitus. With respect to allergic, inflammatory and autoimmune disease, established pre-clinical models may be used and may include: hapten models of dermatitis; collagen-induced arthritis (CIA), adjuvant induced arthritis, cartilage degradation models in the mouse or rat LPS-induced joint inflammation; rat and mouse lung LPS, cytokine, allergen and cigarette smoke-mediated inflammation, lung function and airway remodeling models such as rat tracheal explant model; dextran sodium sulphate (DSS) and trinitrobenzenesulfonic-acid (TNBS) induced colitis in the mouse and rat; behavioral models of learning and memory such as object recognition, fear conditioning, Morris water escape task, passive avoidance test and radial arm maze test; behavioral models of depression such as chronic stress test, tail suspension test, forced swim test, reserpine-mediated hypothermia and yohimbine-induced lethality test.

Compounds of the invention (as defined herein) may inhibit disease induction in these models at doses of less than 20 mg/kg. The Biological Examples below outline some, but not all, of the preclinical models that may be used to support the claims of this patent. For instance, compounds of the examples (described hereinafter) were tested in the Biological examples, and were found to exhibit 50% inhibition of PDE4 at a concentration of 20 μM or below (and more preferably at a concentration of 10 μM or below).

Compounds of the invention (as defined herein) may also be combined with other therapeutic agents that are useful in the treatment of the conditions described herein. For instance, the compounds of the invention (e.g. those hereinbefore defined, but without provisos (B), (C) and (D), or, compounds of the invention, as hereinbefore defined in conjunction with proviso (X^{a}) but without proviso (B)) may be combined with other compounds that may be useful in the treatment of:

i) an inflammatory disorder;
ii) a disorder in which the modulation of intracellular cyclic adenosine 5′-monophosphate levels within a mammal is desired and/or required, which disorder may be an inflammatory disorder;
iii) a disorder associated with pathological conditions that are modulated by inhibiting enzymes associated with secondary cellular messengers (e.g. a cyclic AMP phosphodiesterase; a phosphodiesterase 4; a phosphodiesterase 3; a cyclic GMP phosphodiesterase; or both phosphodiesterase 4 and phosphodiesterase 3), which disorder may be an inflammatory disorder;
iv) transplant rejection in a mammal;
v) uncontrolled cellular proliferation; and/or
vi) a disorder associated with the central nervous system.

According to a further aspect of the invention, there is provided a combination product comprising:
(A) a compound of the invention, as hereinbefore defined but without provisos (B), (C) and (D) (or, compounds of the invention, as hereinbefore defined in conjunction with proviso (V) but without proviso (B)); and
(B) another therapeutic agent that is useful in the treatment of i), ii), iii), iv), v) or yl) above (e.g. a therapeutic agent that is useful in the treatment of an inflammatory disorder),
   wherein each of components (A) and (B) is formulated in admixture with a pharmaceutically-acceptable adjuvant, diluent, carrier or excipient.

Such combination products provide for the administration of a compound of the invention in conjunction with the other therapeutic agent, and may thus be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the other therapeutic agent).

Thus, there is further provided:

(1) a pharmaceutical formulation/composition including a compound of the invention, as hereinbefore defined but without provisos (B), (C) and (D) (or, compounds of the invention, as hereinbefore defined in conjunction with proviso (X^{a}) but without proviso (B)), another therapeutic agent that is useful in the treatment of i), ii), iii), iv), v) or yl) above (e.g. a therapeutic agent that is useful in the treatment of an inflammatory disorder), and a pharmaceutically-acceptable adjuvant, diluent, carrier or excipient; and
(2) a kit of parts comprising components:
(a) a pharmaceutical formulation/composition including a compound of the invention, as hereinbefore defined but without provisos (B), (C) and (D) (or, compounds of the invention, as hereinbefore defined in conjunction with proviso (X^{a}) but without proviso (B)), in admixture with a pharmaceutically-acceptable adjuvant, diluent, carrier or excipient; and
(b) a pharmaceutical formulation/composition including another therapeutic agent that is useful in the treatment of i), ii), iii), iv), v) or vi) above (e.g. a therapeutic agent that is useful in the treatment of an inflammatory disorder) in admixture with a pharmaceutically-acceptable adjuvant, diluent, carrier or excipient,
   which components (a) and (b) are each provided in a form that is suitable for administration in conjunction with the other.

The invention further provides a process for the preparation of a combination product as hereinbefore defined, which process comprises bringing into association a compound of the invention, as hereinbefore defined but without provisos (B), (C) and (D) (or, compounds of the invention, as hereinbefore defined in conjunction with proviso (X^{a}) but without proviso (B)), or a pharmaceutically acceptable derivative (e.g. salt) thereof with another therapeutic agent that is useful in the treatment of i), ii), iii), iv), v) or vi) above (e.g. a therapeutic agent that is useful in the treatment of an inflammatory disorder), and at least one pharmaceutically-acceptable adjuvant, diluent, carrier or excipient.

By “bringing into association”, we mean that the two components are rendered suitable for administration in conjunction with each other.

Thus, in relation to the process for the preparation of a kit of parts as hereinbefore defined, by bringing the two components “into association with” each other, we include that the two components of the kit of parts may be:

(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

### Pharmaceutical Compositions of the Invention and Administration

As stated above, the present invention also relates to pharmaceutical composition containing the compounds of the invention disclosed herein. In one embodiment, the present invention relates to a composition comprising compounds of the invention in a pharmaceutically acceptable carrier and in an amount effective to treat a disease or condition of interest as disclosed herein, such as inflammation and/or rheumatoid arthritis, when administered to an animal, preferably a mammal, most preferably to a human.

Administration of the compounds of the invention (as defined herein), or their pharmaceutically acceptable salts, in pure form or in an appropriate pharmaceutical composition, can be carried out via any of the accepted modes of administration of agents for serving similar utilities.

For instance, compounds of the invention may be administered orally, intravenously, subcutaneously, buccally, rectally, dermally, nasally, tracheally, bronchially, sublingually, by any other parenteral route or via inhalation, in a pharmaceutically acceptable dosage form.

Compounds of the invention may be administered alone, but are preferably administered by way of known pharmaceutical compositions/formulations, including tablets, capsules or elixirs for oral administration, suppositories for rectal administration, sterile solutions or suspensions for parenteral or intramuscular administration, and the like.

The invention further provides a process for the preparation of a pharmaceutical composition/formulation, as hereinbefore defined but without provisos (C) and (D) (or, compounds of the invention, as hereinbefore defined in conjunction with proviso (X^{a})), which process comprises bringing into association a compound of the invention, as hereinbefore defined, or a pharmaceutically acceptable derivative (e.g. salt) thereof, with a pharmaceutically-acceptable adjuvant, carrier, diluent or excipient.

The pharmaceutical compositions so prepared may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, rectal, vaginal, and intranasal. The term parenteral as used herein includes subcutaneous injections, intravenous, intramuscular, intrasternal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see The Science and Practice of Pharmacy, 20th Edition (Philadelphia College of Pharmacy and Science, 2000). The composition to be administered will, in any event, contain a therapeutically effective amount of a compound of the invention, or a pharmaceutically acceptable salt thereof, for treatment of a disease or condition of interest in accordance with the teachings of this invention.

A pharmaceutical composition of the invention (as defined herein) may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example, inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavour enhancer.

In a composition intended to be administered by injection, one or more of a surfactant, preservative, wetting agent, dispersing agent, suspending agent, buffer, stabilizer and isotonic agent may be included.

The liquid pharmaceutical compositions of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

The parenteral preparation can be enclosed in ampoules, disposable syringes or multiple dose vials made of glass or plastic.

Physiological saline is a preferred adjuvant. An injectable pharmaceutical composition is preferably sterile.

A liquid pharmaceutical composition of the invention intended for either parenteral or oral administration should contain an amount of a compound of the invention such that a suitable dosage will be obtained. Typically, this amount is at least 0.01% of a compound of the invention in the composition. When intended for oral administration, this amount may be varied to be between 0.1 and about 70% of the weight of the composition. Preferred oral pharmaceutical compositions contain between about 4% and about 50% of the compound of the invention.

Preferred pharmaceutical compositions and preparations according to the present invention are prepared so that a parenteral dosage unit contains between 0.01 to 10% by weight of the compound prior to dilution of the invention.

The pharmaceutical composition of the invention may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound of the invention from about 0.1 to about 10% w/v (weight per unit volume).

The pharmaceutical composition of the invention may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents.

Alternatively, the active ingredients may be encased in a gelatin capsule. The pharmaceutical composition of the invention in solid or liquid form may include an agent that binds to the compound of the invention and thereby assists in the delivery of the compound. Suitable agents that may act in this capacity include a monoclonal or polyclonal antibody, a protein or a liposome.

The pharmaceutical composition of the invention may consist of dosage units that can be administered as an aerosol. The term aerosol is used to denote a variety of systems ranging from those of colloidal nature to systems consisting of pressurised packages. Delivery may be by a liquefied or compressed gas or by a suitable pump system that dispenses the active ingredients. Aerosols of compounds of the invention may be delivered in single phase, bi-phasic, or triphasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions of the invention may be prepared by methodology well known in the pharmaceutical art. For example, a pharmaceutical composition intended to be administered by injection can be prepared by combining a compound of the invention with sterile, distilled water so as to form a solution. A surfactant may be added to facilitate the formation of a homogeneous solution or suspension. Surfactants are compounds that non-covalently interact with the compound of the invention so as to facilitate dissolution or homogeneous suspension of the compound in the aqueous delivery system.

The compounds of the invention (as defined herein), or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disease or condition; and the subject undergoing therapy. Generally, a therapeutically effective daily dose is (for a 70 kg mammal) from about 0.001 mg/kg (i.e., 0.7 mg) to about 100 mg/kg (i.e., 7.0 gm); preferably a therapeutically effective dose is (for a 70 kg mammal) from about 0.01 mg/kg (i.e., 7 mg) to about 50 mg/kg (i.e., 3.5 gm); more preferably a therapeutically effective dose is (for a 70 kg mammal) from about 1 mg/kg (i.e., 70 mg) to about 25 mg/kg (i.e., 1.75 gm).

The ranges of effective doses provided herein are not intended to be limiting and represent preferred dose ranges. However, the most preferred dosage will be tailored to the individual subject, as is understood and determinable by one skilled in the relevant arts. (see, e.g., Berkow et al., eds., The Merck Manual, 16th edition, Merck and Co., Rahway, N.J., 1992; Goodmanetna., eds., Goodman and Cilman's The Pharmacological Basis of Therapeutics, 10th edition, Pergamon Press, Inc., Elmsford, N.Y., (2001); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985); Osolci al., eds., Remington's Pharmaceutical Sciences, 18th edition, Mack Publishing Co., Easton, Pa. (1990); Katzung, Basic and Clinical Pharmacology, Appleton and Lange, Norwalk, Conn. (1992)).

The total dose required for each treatment can be administered by multiple doses or in a single dose over the course of the day, if desired. Generally, treatment is initiated with smaller dosages, which are less than the optimum dose of the compound.

Thereafter, the dosage is increased by small increments until the optimum effect under the circumstances is reached. The diagnostic pharmaceutical compound or composition can be administered alone or in conjunction with other diagnostics and/or pharmaceuticals directed to the pathology, or directed to other symptoms of the pathology. The recipients of administration of compounds and/or compositions of the invention can be any vertebrate animal, such as mammals. Among mammals, the preferred recipients are mammals of the Orders Primate (including humans, apes and monkeys), Arteriodactyla (including horses, goats, cows, sheep, pigs), Rodenta (including mice, rats, rabbits, and hamsters), and Carnivora (including cats, and dogs). Among birds, the preferred recipients are turkeys, chickens and other members of the same order. The most preferred recipients are humans.

For topical applications, it is preferred to administer an effective amount of a pharmaceutical composition according to the invention to target area, e.g., skin surfaces, and the like. This amount will generally range from about 0.0001 mg to about 1 g of a compound of the invention per application, depending upon the area to be treated, whether the use is diagnostic, prophylactic or therapeutic, the severity of the symptoms, and the nature of the topical vehicle employed. A preferred topical preparation is an ointment, wherein about 0.001 to about 50 mg of active ingredient is used per cc of ointment base. The pharmaceutical composition can be formulated as transdermal compositions or transdermal delivery devices (“patches”). Such compositions include, for example, a backing, active compound reservoir, a control membrane, liner and contact adhesive. Such transdermal patches may be used to provide continuous pulsatile, or on demand delivery of the compounds of the present invention as desired.

The compositions of the invention can be formulated so as to provide quick, sustained or delayed release of the active ingredient after administration to the patient by employing procedures known in the art. Controlled release drug delivery systems include osmotic pump systems and dissolutional systems containing polymer-coated reservoirs or drug-polymer matrix formulations. Examples of controlled release systems are given in U.S. Pat. Nos. 3,845,770 and 4,326,525 and in P. J. Kuzma et al, Regional Anesthesia 22 (6): 543-551 (1997), all of which are incorporated herein by reference.

The compositions of the invention can also be delivered through intra-nasal drug delivery systems for local, systemic, and nose-to-brain medical therapies. Controlled Particle Dispersion (CPD)™ technology, traditional nasal spray bottles, inhalers or nebulizers are known by those skilled in the art to provide effective local and systemic delivery of drugs by targeting the olfactory region and paranasal sinuses.

The invention also relates to an intravaginal shell or core drug delivery device suitable for administration to the human or animal female. The device may be comprised of the active pharmaceutical ingredient in a polymer matrix, surrounded by a sheath, and capable of releasing the compound in a substantially zero order pattern on a daily basis similar to devises used to apply testosterone as desscribed in PCT Patent No. WO 98/50016. Current methods for ocular delivery include topical administration (eye drops), subconjunctival injections, periocular injections, intravitreal injections, surgical implants and iontophoresis (uses a small electrical current to transportionized drugs into and through body tissues). Those skilled in the art would combine the best suited excipients with the compound for safe and effective intra-occular administration. The most suitable route will depend on the nature and severity of the disease or condition being treated. Those skilled in the art are also familiar with determining administration methods (oral, intravenous, inhalation, subcutaneous, rectal etc.), dosage forms, suitable pharmaceutical excipients and other matters relevant to the delivery of the compounds to a subject in need thereof.

Compounds of the invention may have the advantage that they are effective inhibitors (and hence particularly effective in the treatment of the conditions described herein), and in particular effective PDE inhibitors (and especially effective PDE4 inhibitors).

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise.

### EXAMPLES

All compounds of the invention as prepared above which exist in free base or acid form may be converted to their pharmaceutically acceptable salts by treatment with the appropriate inorganic or organic base or acid. Salts of the compounds prepared above may be converted to their free base or acid form by standard techniques. It is understood that all polymorphs, amorphous forms, anhydrates, hydrates, solvates and salts of the compounds of the invention are intended to be within the scope of the invention. Furthermore, all compounds of the invention which contain an ester group can be converted to the corresponding acid by methods known to one skilled in the art or by methods described herein.

The following specific Synthetic Preparations (for the preparation of starting materials and intermediates) and Synthetic Examples (for the preparation of the compounds of the invention) and the Biological Examples (for the assays used to demonstrate the utility of the compounds of the invention) are provided as a guide to assist in the practice of the invention, and are not intended as a limitation on the scope of the invention. Where one or more NMR data are given for a particular compound, each NMR may represent a single stereoisomer, a non-racemic mixture of stereoisomers or a racemic mixture of the stereoisomers of the compound.

### Synthetic Preparation of Intermediates of Formula III

### Preparation Route 1

A. To a stirred mixture of absolute EtOH (28.5 mL) and chloroform (31 mL) at 0° C. was added acetyl chloride (31.5 mL) dropwise. After 5 min., a solution of chloroacetonitrile (8 mL, 126 mmol) in chloroform (31 mL) was added. The mixture was stirred at 0° C. for 2 hours 35 min. and then was concentrated by rotary evaporation. The resulting white solid was washed with ether, and dried to give ethyl 2-chloroacetimidate hydrochloride.

B. To a stirred suspension of 2-aminophenol (365 mg, 3.35 mmol) in dichloromethane (7 mL) at 0° C. was added compound ethyl 2-chloroacetimidate hydrochloride (798 mg, 5.05 mmol) portion-wise. After 1 hour 25 min. at 0° C., the mixture was stirred at ambient temperature overnight. The mixture was filtered through Celite and the filtrate was concentrated. The residue was purified by column (hexanes/EtOAc, 7/3) to afford 2-(chloromethyl)benzoxazole as a pale oil (533 mg, 95%).

C. To a stirred solution of 2-(chloromethyl)benzoxazole (533 mg, 3.2 mmol) in acetone (23 mL) was added KI (1.68 g, 10.1 mmol) and the mixture was stirred at ambient temperature overnight. The mixture was concentrated and the residue was diluted with dichloromethane, washed with brine, dried and concentrated. The residue was purified by column (hexanes/EtOAc, 7/3) to yield 2-(iodomethyl)benzoxazole as a brown solid (746 mg, 90%).

D. In a similar manner utilizing the appropriately substituted starting materials, the following compounds of formula (2) were prepared:
2-(iodomethyl)-5-(trifluoromethyl)benzoxazole;
5-cyano-2-(iodomethyl)benzoxazole;
5-fluoro-2-(iodomethyl)benzoxazole;
2-(iodomethyl)-5-methoxybenzoxazole; and
7-fluoro-2-(iodomethyl)benzoxazole.

### Preparation Route 2

A. To a stirred mixture of o-cresol (1.0 g, 9.25 mmol) and Bi(NO₃)₃5H₂O (2.24 g, 4.62 mmol) was added acetone (9 mL) at once. The resulting mixture was stirred at ambient temperature for 8 min. and then filtered through Celite. The Celite was washed with dichloromethane several times and the filtrate was concentrated. The residue was separated by column (hexanes/EtOAc, 8/2) to give 2-methyl-6-nitrophenol as a yellow solid (484 mg, 34%).

B. A mixture of 2-methyl-6-nitrophenol (542 mg, 3.54 mmol), 10% Pd/C (54 mg) in EtOAc/EtOH (5.5 mL/11 mL) was stirred under hydrogen at atmospheric pressure overnight. The mixture was filtered through Celite and the filtrate was concentrated to give 2-amino-6-methylphenol as a brown solid (373 mg, 85%). The product was then converted to 2-(iodomethyl)-7-methylbenzoxazole a manner similar as described above in Preparation Route 1.

C. In a similar manner utilizing the appropriately substituted starting materials, additional compounds of formula III are prepared.

### Preparation Route 3

A. To a stirred solution of 2-fluoroaniline (3 mL, 31.1 mmol) in a mixture of sulfuric acid (5.4 mL) and TFA (27 mL) at 0° C. was added a solution of sodium nitrite (2.79 g, 40.5 mmol) in water (27 mL) over 5 min. After 35 min. at 0° C., a solution of sodium azide (3.52 g, 54.2 mmol) was added dropwise over 40 min. The resulting mixture was stirred at 0° C. for another 1 hour 10 min. and then was extracted with ether. The combined ether extracts were washed with saturated aqueous NaHCO₃, brine, dried and concentrated to give the crude 1-azido-2-fluorobenzene. The crude product was then heated at 140° C. in acetic anhydride (30 mL) overnight. The reaction mixture was cooled to ambient temperature and then concentrated. The residue was purified by column (hexanes/EtOAc, 6/4) to yield 2-acetamido-3-fluorophenyl acetate as brown oil (3.17 g, 48% over two steps). This product was then treated with saturated NaHCO₃ (50 mL) in MeOH (50 mL) at ambient temperature for about 5.5 hours and then most of MeOH was removed by rotary evaporation. The aqueous residue was acidified by diluted HCl solution to pH 2-3 and then extracted with EtOAc. The combined EtOAc extracts were washed with saturated NaHCO₃ until pH neutral and then it was washed with brine, dried and concentrated. The crude product was crystallized to give N-(2-fluoro-6-hydroxyphenyl)acetamide (451 mg, 18%). The acetamide was then treated with 5% HCl (20 mL) at 110° C. for 0.5 hours. The solvents were removed and the residue was suspended in saturated NaHCO₃ and EtOAc. The organic layer was separated and the aqueous phase was extracted with EtOAc. The combined EtOAc extracts were washed with brine, dried and concentrated to yield 2-amino-3-fluorophenol as a brown solid (353 mg, 74%). The mother liquor from N-(2-fluoro-6-hydroxyphenyl)acetamide was treated with 5% HCl in the same manner to give another crop of 2-amino-3-fluorophenol (415 mg), bringing the total overall yield to 19% over four steps. In a similar manner as described above in Preparation Route 1,2-amino-3-fluorophenol was converted to 4-fluoro-2-(iodomethyl)benzoxazole.

B. In a similar manner utilizing the appropriately substituted starting materials, additional compounds of formula III are prepared.

### Preparation Route 4

A. To a stirred solution of sodium nitrate (1.56 g, 18.4 mmol) in 3M sulfuric acid (16.5 mL) at ambient temperature was added a solution of 3-(trifluoromethyl)phenol (2 mL, 16.7 mmol) in dichloromethane (35 mL), followed by a few grains of sodium nitrite. The mixture was stirred at ambient temperature overnight and then diluted with EtOAc, washed with brine, dried and concentrated. The crude product was purified by column (hexanes/EtOAc, 6/4 to 5/5) to yield 2-nitro-3-(trifluoromethyl)phenol as a yellow solid (1.29 g, 37%).

B. A mixture of 2-nitro-3-(trifluoromethyl)phenol (1.426 g, 6.9 mmol) and 10% Pd/C (142 mg) in ethanol (10 mL) was stirred under hydrogen at atmosphere overnight. The catalyst was removed by filtration through Celite and the filtrate was concentrated to give 2-amino-3-(trifluoromethyl)phenol (868 mg, 71%). This material was then converted to 2-(iodomethyl)-4-(trifluoromethyl)-benzoxazole in a similar manner as in Preparation Route 1.

### Preparation Route 5

A. To a stirred solution of sodium nitrate (1.24 g, 14.6 mmol) in 3M sulfuric acid (13.2 mL) at ambient temperature was added a solution of 3-hydroxybenzonitrile (1.6 g, 13.4 mmol) in dichloromethane (28 mL), followed by a few grains of sodium nitrite. The mixture was stirred at ambient temperature overnight and then diluted with EtOAc, washed with brine, dried and concentrated. The crude product was purified by column (hexanes/EtOAc, 5/5 to 2/8) to yield 3-hydroxy-2-nitrobenzonitrile (1.31 g, mixed with another isomer).

B. A mixture of 3-hydroxy-2-nitrobenzonitrile (1.31 g) and 10% Pd/C (131 mg) in ethanol (9 mL) and EtOAc (18 mL) was stirred under hydrogen at atmosphere overnight. The catalyst was removed by filtration through Celite and the filtrate was concentrated. The residue was purified by column (hexanes/EtOAc, 6/4) to give 2-amino-3-hydroxybenzonitrile (398 mg, 37%). This material was then converted to 4-cyano-2-(iodomethyl)benzoxazole in a similar manner as in Preparation Route 1.

### Preparation Route 6

A. A mixture of 2-nitrobenzene-1,3-diol (625 mg, 4.0 mmol) and 10% Pd/C (62 mg) was stirred in ethanol (20 mL) under hydrogen at atmospheric pressure for 6 hours. The catalyst was removed by filtration and the filtrate was concentrated to give 2-aminobenzene-1,3-diol as a crystalline solid (542 mg, crude). Then it was reacted with ethyl 2-chloroacetimidate hydrochloride (890 mg, 5.63 mmol) in a mixture of THF (4 mL) and dichloromethane (15.6 mL) at ambient temperature overnight. The reaction mixture was filtered through Celite and washed with dichloromethane. The filtrate was concentrated and the residue was purified by column (hexanes/EtOAc, 6/4) to give 2-(chloromethyl)-4-hydroxybenzoxazole as a crystalline solid (413 mg, 56% over two steps). This compound was then treated with methyl sulfate (0.32 mL, 3.38 mmol) and K₂CO₃ (373 mg, 2.7 mmol) in acetone (9.1 mL) at ambient temperature overnight. The mixture was then diluted with EtOAc, washed with saturated aqueous NaHCO₃, brine, dried and concentrated. The residue was purified by column (hexanes/EtOAc, 6/4) to give 2-(chloromethyl)-4-methoxybenzoxazole as oil. This compound was then stirred with KI (1.115 g, 6.73 mmol) in acetone (16 mL) overnight and then the mixture was concentrated. The residue was suspended in dichloromethane and washed with brine, dried and concentrated. The residue was purified by column (hexanes/EtOAc, 7/3) to give 2-(iodomethyl)-4-methoxybenzoxazole (358 mg, 55% over two steps).

### Preparation Route 7

A. 2-aminobenzenethiol (3, R₁=H, Z=S) (419 mg, 3.35 mmol) and ethyl 2-dhloroacetimidate hydrochloride (798 mg, 5.05 mmol) were reacted in a similar manner as described above in Preparation Route 1, to give 2-(chloromethyl)benzothiazole (175 mg, 28%) as a yellow oil.

B. 2-(Chloromethyl)benzothiazole (365 mg, 2.0 mmol) was treated with KI (1.05 g, 6.35 mmol) in acetone (14 mL) in a similar manner as in Synthetic Preparation 1 to give 2-(iodomethyl)benzothiazole as a brownish solid (417 mg, 76%)

### Preparation Route 8

A. A mixture of 2-chloroacetic acid (900 mg, 9.52 mmol) and benzene-1,2-diamine (908 mg, 8.4 mmol) in 6N HCl was refluxed overnight. The mixture was cooled in ice-water and saturated aqueous ammonia was added with stirring until the product crashed out. The mixture was filtered and the solid product was washed with water and dried to yield 2-(chloromethyl)-benzimidazole as a pale yellow powder (1.05 g, 75%).

B. A mixture of 2-(chloromethyl)-benzimidazole (523 mg, 3.14 mmol), di-tbutyldicarbonate (1.37 g, 6.28 mmol), and triethylamine (0.88 mL, 6.28 mmol) in dichloromethane (16 mL) was cooled to 0° C., and catalytic amount of DMAP was added. After 2 hours, the mixture was diluted with EtOAc, washed with saturated NaHCO₃, brine, dried, filtered, and concentrated. The crude product was purified with Flashmaster (5% to 10% EtOAc/hexanes) to give t-butyl 2-(chloromethyl)benzimidazole-1-carboxylate as a crystalline solid (760 mg, 91%).

C. t-Butyl 2-(chloromethyl)-benzimidazole-1-carboxylate (755 mg, 2.83 mmol) was heated with KI (1.4 g, 8.5 mmol) in acetone (15 mL) at 60° C. for 2 hours. After cooling to ambient temperature, the mixture was filtered and the filtrate was concentrated. The residue was purified with Flashmaster (5 to 10% EtOAc in hexanes) to yield 2-(iodomethyl)-benzoimidazole-1-carboxylate as a brown solid (954 mg, 94%).

D. In a similar manner utilizing the appropriately substituted starting materials, the following compounds of formula III were prepared:
2-(iodomethyl)-benzimidazole;
2-(iodomethyl)-5,6-dimethyl-benzimidazole; and
5,6-dichloro-2-(iodomethyl)-benzimidazole.

### Preparation Route 9

A. A mixture of 2-chloroacetic acid (850 mg, 9.0 mmol) and N-methyl-1,2-phenylenediamine (1.0 g, 8.2 mmol) in 6N HCl (10 mL) was heated at 110° C. overnight. The mixture was cooled in ice-water and saturated aqueous ammonia was added with stirring until the product crashed out. The mixture was filtered and the solid product was washed with water. The solid was purified with Flashmaster (5% to 15% to 50% EtOAc in hexanes) to afford 2-(chloromethyl)-1-methyl-benzimidazole as a white crystalline solid (878 mg, 59%).

B. 2-(Chloromethyl)-1-methyl-benzimidazole (872 mg, 4.83 mmol) was heated with KI (2.4 g, 14.5 mmol) in acetone (24 mL) at 60° C. for 2 hours. After cooling to ambient temperature, the mixture was filtered and the filtrate was concentrated. The residue was purified with Flashmaster (5, 15, 50% EtOAc in hexanes) to yield 2-(iodomethyl)-1-methyl-benzimidazole (328 mg, 25%).

### Preparation Route 10

A. To a stirred solution of benzofuran-2-carbaldehyde (0.39 mL, 3.2 mmol) in MeOH (10 mL) at 0° C. was added NaBH₄ (60 mg, 1.6 mmol) portion wise. The mixture was stirred at 0° C. for 15 min. and then at ambient temperature for 1 hour 15 min. A small amount of water was added and the mixture was stirred for 15 min before rotary evaporation. The residue was purified to afford 2-(hydroxymethyl)benzofuran (479 mg, pale oil, 100%).

B. To a stirred solution of 2-(hydroxymethyl)benzofuran (479 mg, 3.2 mmol) in ether (10 mL) at 0° C. was added PBr₃ (0.31 mL, 3.2 mmol) dropwise. After stirring at 0° C. for 50 min., the mixture was stirred at ambient temperature for 2 hour 45 min. The mixture was diluted with water and the aqueous layer was separated and extracted with ether. The organic extracts were combined, washed with saturated NaHCO₃, brine, dried, and concentrated to give 2-(bromomethyl)benzofuran as pale oil (391 mg, 57%).

### Preparation Route 11

A. A mixture of 2-hydroxy-5-methoxybenzaldehyde (1.00 g, 6.57 mmol), ethyl 2-chloroacetate (0.77 mL, 7.23 mmol), and K₂CO₃ (1.82 g, 13.1 mmol) in DMF (13 mL) was heated at 90° C. for 2 days. The reaction mixture was cooled to ambient temperature, diluted with water, and extracted with ether. The organic extracts were combined and washed with brine, dried, and concentrated. The residue was purified with Flashmaster (5% to 10% EtOAc in hexanes) to give ethyl 5-methoxybenzofuran-2-carboxylate as a white crystalline solid (762 mg, 53%).

B. To a stirred solution of ethyl 5-methoxybenzofuran-2-carboxylate (755 mg, 3.43 mmol) in ether (14.6 mL) at 0° C. was added LiAlH₄ in THF (1.0 M, 2.6 mL). The reaction was quenched with saturated aqueous NaHCO₃, diluted with water and extracted with EtOAc. The organic extracts were washed with brine, dried, and concentrated. The residue was purified with Flashmaster (5% to 15% to 30% EtOAc in hexanes) to yield (5-methoxybenzofuran-2-yl)methanol as a crystalline solid (547 mg, 90%).

C. To a cooled solution of (5-methoxybenzofuran-2-yl)methanol (543 mg, 3.05 mmol), carbon tetrabromide (162 mg, 4.88 mmol) in dichloromethane (15 mL) was added PPh₃ (960 mg, 3.66 mmol). After 1 hr, the reaction mixture was concentrated and purified with Flashmaster (5% to 10% EtOAc in hexanes) to give 2-(bromomethyl)-5-methoxybenzofuran as a pale yellow solid (644 mg, 88%).
D. In a similar manner utilizing the appropriately substituted starting materials, the following compounds of formula III were prepared:
2-(bromomethyl)-7-fluorobenzofuran;
2-(bromomethyl)-5-(trifluoromethoxy)benzofuran; and
2-(bromomethyl)-7-methoxybenzofuran.

### Preparation Route 12

A. To a stirred solution of benzothiophene-3-carbaldehyde (414 mg, 2.05 mmol) in MeOH (10 mL) at 0° C. was added NaBH₄ (75.7 mg, 2.0 mmol). After 5 min., the mixture was stirred at ambient temperature for about 3.5 hours, and then quenched by addition of water. The reaction mixture was then worked up with standard procedure to give benzothien-3-ylmethanol as pale oil (400 mg, 97%).

B. To a stirred solution of benzothien-3-ylmethanol (400 mg, 2.44 mmol) in ether (12 mL) at 0° C. was added PBr₃ (0.23 mL, 2.42 mmol) dropwise. The mixture was stirred at 0° C. for 30 min. and then at ambient temperature for 45 min. The reaction mixture was poured into a mixture of ice water and ether. The organic phase was separated and washed with brine, dried and concentrated to give 3-(bromomethyl)benzothiophene as a pale solid (404 mg, 73%).

### Preparation Route 13

A. To a stirred suspension of indole-3-carbaldehyde (290 mg, 2 mmol) in acetonitrile (5 mL) was added DMAP (19.6 mg) and Boc₂O (458 mg, 2.1 mmol). The mixture was stirred at ambient temperature overnight, diluted with EtOAc, washed with brine, dried and concentrated. The crude product (467 mg) was suspended in MeOH (10 mL), cooled to 0° C. and treated with NaBH₄ (75 mg, 2 mmol). After 10 min., the reaction mixture was stirred at ambient temperature for 1 hr, quenched with water, and worked up with standard procedure to give crude t-butyl 3-(hydroxymethyl)-indole-1-carboxylate (469 mg) as a gum.

B. To a stirred solution of t-butyl 3-(hydroxymethyl)-indole-1-carboxylate (123 mg, 0.5 mmol), PPh₃ (170 mg, 0.65 mmol) in dichloromethane (5 mL) was added carbon tetrabromide (199 mg, 0.6 mmol). The reaction mixture was stirred at ambient temperature for 8 hrs and additional PPh₃ (20 mg) was added. After another 16 hours, additional carbon tetrabromide (30 mg) was added and the reaction was continued overnight. The solvent was removed by rotary evaporation and the residue was purified by a short column (hexanes/EtOAc, 7/3) to give t-butyl 3-(bromomethyl)-indole-1-carboxylate as oil (119 mg, 77%).

### Preparation Route 14

A mixture of furan-2-ylmethanol (0.2 mL, 2.3 mmol), PPh₃ (784 mg, 3.0 mmol), and carbon tetrabromide (915 mg, 2.76 mmol) in dichloromethane (15 mL) was stirred at ambient temperature overnight. The mixture was poured into hexanes (˜200 mL) and the resulting precipitate was removed by vacuum filtration through a Celite pad. The filtrate was concentrated and any additional precipitate was filtered off. This process was repeated until no precipitate formed upon concentration. The filtrate, which contained 2-(bromomethyl)furan, was used without further purification.

### Preparation Route 15

To a stirred solution of thien-2-ylmethanol (456.7 mg, 4 mmol) in ether at 0° C. was added PBr₃ (0.38 mL) dropwise. The reaction mixture was stirred at 0° C. for 1 hour 40 min. and then at ambient temperature for 35 min. before poured into a mixture of ice water and ether. The organic phase was separated, washed with brine, dried and concentrated to yield 2-(bromomethyl)thiophene as pale oil (577 mg, 81%).

### Preparation Route 16

A. To a stirred solution of (6-methylpyridin-2-yl)methanol (640 mg, 5.2 mmol) and carbon tetrabromide (2.77 g, 8.35 mmol) in dichloromethane (12 mL) at 0° C. was added PPh₃ (0.4 g×4, 6.1 mmol) at 2 min intervals. The reaction was continued for total of 30 min. and then the mixture was directly purified with Flashmaster using 10% EtOAc in hexanes to give 2-(bromomethyl)-6-methylpyridine as a crystalline solid (675 mg, 70%).

B. Alternatively, a mixture of pyridine-3-methanol (4.4 mL, 45.7 mmol) and aqueous (48%) hydrobromic acid (45 mL, 398 mmol) was heated at reflux for 5.5 hours and concentrated in vacuo. The residue was re-crystallized from absolute ethanol twice to afford 3-(bromomethyl)pyridine hydrobromide (6.22 g, 54%) as a white solid, which was converted to the free base prior to further use.

C. In a similar manner utilizing the appropriately substituted starting materials, the following compound of formula III was prepared:
4-(bromomethyl)pyridine hydrobromide, 33% yield.

### Preparation Route 17

A mixture of 1-(chloromethyl)-benzotriazole and KI (747 mg, 4.5 mmol) was stirred in acetone (10 mL) at ambient temperature overnight. The solvent was removed by rotary evaporation and the residue was diluted with dichloromethane, washed with brine, dried and concentrated. The crude product was purified by column (hexanes/EtOAc, 7/3) to give 1-(iodomethyl)-benzotriazole as a yellow solid (344 mg, 88%).

### Example 1

A. To a stirred solution of (S)-tert-butyl 5-(3-(cyclopentyloxy)-4-methoxyphenyl)-2-oxopiperidine-1-carboxylate (292 mg, 0.75 mmol) in THF (8 mL) at −78° C. was added LDA (0.7M, 1.3 mL, prepared from diisopropylamine (0.5 mL), 1.6M n-BuLi (2 mL) and THF (2.1 mL) at 0° C. for 50 min) dropwise and the resulting mixture was stirred for 1 hour 15 min. at −78° C. A solution of 2-(iodomethyl)benzoxazole (252 mg, 0.975 mmol) in THF (1.5 mL) was added dropwise and the resulting mixture was stirred at −78° C. for one hour and then allowed to warm to ˜−40° C. in about 1.5 hour. Saturated NH₄Cl (2 mL) was added and the mixture was diluted with EtOAc, washed with brine, dried and concentrated. Purification by column chromatography (hexanes/EtOAc, 6/4) afforded an isomeric mixture of products, (5S)-tert-butyl 3-(benzoxazol-2-ylmethyl)-5-(3-(cyclopentyloxy)-4-methoxyphenyl)-2-oxopiperidine-1-carboxyl-ate (376 mg, 96%). This isomeric mixture of products was then treated with TFA (0.5 mL) in dichloromethane (20 mL) at 0° C. for 1 hour and then diluted with toluene (˜20 mL). The solvents were removed by rotary evaporation and the residue was separated by column (EtOAc) to give (3R,5S)-3-(benzoxazol-2-ylmethyl)-5-(3-(cyclopentyloxy)-4-methoxyphenyl)piperidin-2-one, MW 420.50; (159 mg, 53%) and (3S,5S)-3-(benzoxazol-2-ylmethyl)-5-(3-(cyclopentyloxy)-4-methoxyphenyl)piperidin-2-one, MW 420.50 (42 mg, 14%).

B. In a similar manner (to A above) utilizing the appropriately substituted starting material and intermediate compound of formula III, the following compounds were made:
(3R,5S)-3-(2-benzofurylmethyl)-5-(3-(cyclopentyloxy)-4-methoxyphenyl)pip-eridin-2-one, MW 419.51;
(3S,5S)-3-(2-benzofurylmethyl)-5-(3-(cyclopentyloxy)-4-methoxyphenyl)pip-eridin-2-one, MW 419.51;
(3R,5S)-5-(3-(cyclopentyloxy)-4-methoxyphenyl)-3-(2-thienylmethyl)piperidin-2-one, MW 385.52;
(3S,5S)-5-(3-(cyclopentyloxy)-4-methoxyphenyl)-3-(2-thienylmethyl)piperidin-2-one, MW 385.52;
(3R,5S)-5-(3-(cyclopentyloxy)-4-methoxyphenyl)-3-(2-furylmethyl)piperidin-2-one, MW 369.45;
(3S,5S)-5-(3-(cyclopentyloxy)-4-methoxyphenyl)-3-(2-furylmethyl)piperidin-2-one, MW 369.45;
(3S,5S)-5-(3-(cyclopentyloxy)-4-methoxyphenyl)-3-(3-indolylmethyl)piperidin-2-one, MW 418.53;
(3R,5S)-3-(3-benzothienylmethyl)-5-(3-(cyclopentyloxy)-4-methoxyphenyl)piperidin-2-one, MW 435.58;
(3S,5S)-3-(3-benzothienylmethyl)-5-(3-(cyclopentyloxy)-4-methoxyphenyl)piperidin-2-one, MW 435.58;
(3R,5S)-3-(2-benzothiazolylmethyl)-5-(3-(cyclopentyloxy)-4-methoxyphenyl)piperidin-2-one, MW 436.57;
(3S,5S)-3-(2-benzothiazolylmethyl)-5-(3-(cyclopentyloxy)-4-methoxyphenyl)piperidin-2-one, MW 436.57;
(3R,5S)-5-(3-(cyclopentyloxy)-4-methoxyphenyl)-3-(5-methyl-2-benzoxazolylmethyl)piperidin-2-one, MW 434.53;
(3S,5S)-5-(3-(cyclopentyloxy)-4-methoxyphenyl)-3-(5-methyl-2-benzoxazolylmethyl)piperidin-2-one, MW 434.53;
(3R,5S)-3-(6-chloro-2-benzoxazolylmethyl)-5-(3-(cyclopentyloxy)-4-methoxyphenyl)piperidin-2-one, MW 454.95;
(3S,5S)-3-(6-chloro-2-benzoxazolylmethyl)-5-(3-(cyclopentyloxy)-4-methoxyphenyl)piperidin-2-one, MW 454.95;
(3R,5S)-5-(3-(cyclopentyloxy)-4-methoxyphenyl)-3-(4-methyl-2-benzoxazolylmethyl)piperidin-2-one, MW 434.53;
(3S,5S)-5-(3-(cyclopentyloxy)-4-methoxyphenyl)-3-(4-methyl-2-benzoxazolylmethyl)piperidin-2-one, MW 434.53;
(3R,5S)-5-(3-(cyclopentyloxy)-4-methoxyphenyl)-3-(6-methyl-2-benzoxazolylmethyl)piperidin-2-one, MW 434.53;
(3S,5S)-5-(3-(cyclopentyloxy)-4-methoxyphenyl)-3-(6-methyl-2-benzoxazolylmethyl)piperidin-2-one, MW 434.53;
(3R,5S)-3-(1-benzotriazolylmethyl)-5-(3-(cyclopentyloxy)-4-methoxyphenyl)piperidin-2-one, MW 420.51;
(3S,5S)-3-(1-benzotriazolylmethyl)-5-(3-(cyclopentyloxy)-4-methoxyphenyl)piperidin-2-one, MW 420.51;
(3R,5S)-5-(3-(cyclopentyloxy)-4-methoxyphenyl)-3-(5-nitro-2-benzoxazolylmethyl)piperidin-2-one, MW 465.50;
(3S,5S)-5-(3-(cyclopentyloxy)-4-methoxyphenyl)-3-(5-nitro-2-benzoxazolylmethyl)piperidin-2-one, MW 465.50;
(3R,5S)-3-(5-cyano-2-benzoxazolylmethyl)-5-(3-cyclopentyloxy-4-methoxyphenyl)piperidin-2-one, MW 445.51;
(3S,5S)-3-(5-cyano-2-benzoxazolylmethyl)-5-(3-cyclopentyloxy-4-methoxyphenyl)piperidin-2-one, MW 445.51;
(3R,5S)-5-(3-cyclopentyloxy-4-methoxyphenyl)-3-(5-fluoro-2-benzoxazolylmethyl)piperidin-2-one, MW 438.49;
(3S,5S)-5-(3-cyclopentyloxy-4-methoxyphenyl)-3-(5-fluoro-2-benzoxazolylmethyl)piperidin-2-one, MW 438.49;
(3R,5S)-5-(3-cyclopentyloxy-4-methoxyphenyl)-3-(5-trifluoromethyl-2-benzoxazolylmethyl)piperidin-2-one, MW 488.50;
(3S,5S)-5-(3-cyclopentyloxy-4-methoxyphenyl)-3-(5-trifluoromethyl-2-benzoxazolylmethyl)piperidin-2-one, MW 488.50;
(3S,5S)-5-(3-cyclopentyloxy-4-methoxyphenyl)-3-(7-methyl-2-benzoxazolylmethyl)piperidin-2-one, MW 434.53;
(3R,5S)-5-(3-cyclopentyloxy-4-methoxyphenyl)-3-(7-methyl-2-benzoxazolylmethyl)piperidin-2-one, MW 434.53;
(3S,5S)-5-(3-cyclopentyloxy-4-methoxyphenyl)-3-(5-methoxy-2-benzoxazolylmethyl)piperidin-2-one, MW 450.53;
(3R,5S)-5-(3-cyclopentyloxy-4-methoxyphenyl)-3-(5-methoxy-2-benzoxazolylmethyl)piperidin-2-one, MW 450.53;
(3S,5S)-5-(3-cyclopentyloxy-4-methoxyphenyl)-3-(4-methoxy-2-benzoxazolylmethyl)piperidin-2-one, MW 450.53;
(3R,5S)-5-(3-cyclopentyloxy-4-methoxyphenyl)-3-(4-methoxy-2-benzoxazolylmethyl)piperidin-2-one, MW 450.53;
(3S,5S)-5-(3-cyclopentyloxy-4-methoxyphenyl)-3-(4-fluoro-2-benzoxazolylmethyl)piperidin-2-one, MW 438.50;
(3R,5S)-5-(3-cyclopentyloxy-4-methoxyphenyl)-3-(4-fluoro-2-benzoxazolylmethyl)piperidin-2-one, MW 438.50;
(3S,5S)-5-(3-cyclopentyloxy-4-methoxyphenyl)-3-(7-fluoro-2-benzoxazolylmethyl)piperidin-2-one, MW 438.50;
(3R,5S)-5-(3-cyclopentyloxy-4-methoxyphenyl)-3-(7-fluoro-2-benzoxazolylmethyl)piperidin-2-one, MW 438.50;
(3S,5S)-3-(4-cyano-2-benzoxazolylmethyl)-5-(3-cyclopentyloxy-4-methoxyphenyl)piperidin-2-one, MW 445.52;
(3R,5S)-3-(4-cyano-2-benzoxazolylmethyl)-5-(3-cyclopentyloxy-4-methoxyphenyl)piperidin-2-one, MW 445.52;
(3S,5S)-3-(7-chloro-2-benzoxazolylmethyl)-5-(3-cyclopentyloxy-4-methoxyphenyl)piperidin-2-one, MW 454.95;
(3R,5S)-3-(7-chloro-2-benzoxazolylmethyl)-5-(3-cyclopentyloxy-4-methoxyphenyl)piperidin-2-one, MW 454.95;
(3S,5S)-5-(3-cyclopentyloxy-4-methoxyphenyl)-3-(4-trifluoromethyl-2-benzoxazolylmethyl)piperidin-2-one, MW 488.51;
(3R,5S)-5-(3-cyclopentyloxy-4-methoxyphenyl)-3-(4-trifluoromethyl-2-benzoxazolylmethyl)piperidin-2-one, MW 488.51;
(3S,5S)-5-(3-cyclopentyloxy-4-methoxyphenyl)-3-(5-methoxy-2-benzofurylmethyl)piperidin-2-one, MW 449.54;
(3R,5S)-5-(3-cyclopentyloxy-4-methoxyphenyl)-3-(5-methoxy-2-benzofurylmethyl)piperidin-2-one, MW 449.54;
(3R,5S)-5-(3-cyclopentyloxy-4-methoxyphenyl)-3-(7-methoxy-2-benzofurylmethyl)piperidin-2-one, MW 449.54;
(3S,5S)-5-(3-cyclopentyloxy-4-methoxyphenyl)-3-(7-methoxy-2-benzofurylmethyl)piperidin-2-one , MW 449.54;
(3R,5S)-5-(3-cyclo pentyloxy-4-methoxyphenyl)-3-(741 uoro-2-benzofurylmethyl)piperidin-2-one, MW 437.5; and
(3S,5S)-5-(3-cyclopentyloxy-4-methoxyphenyl)-3-(7-fluoro-2-benzofurylmethyl)-piperidin-2-one, MW 437.5.

### Example 2

A. Alkylation of (S)-tert-butyl 5-(3-(cyclopentyloxy)-4-methoxyphenyl)-2-oxopiperidine-1-carboxylate (292 mg, 0.75 mmol) with tert-butyl 2-(iodomethyl)-5,6-dimethyl-benzimidazole-1-carboxylate (326 mg, 0.85 mmol) in a similar manner as in Example 1 above yielded tert-butyl 2-(((5S)-1-(tert-butoxycarbonyl)-5-(3-(cyclopentyloxy)-4-methoxyphenyl)-2-oxopiperidin-3-yl)-methyl)-5,6-dimethylbenzimidazole-1-carboxylate (441 mg, 68%). Treatment with TFA/dichloromethane in a similar manner gave tert-butyl 2-(((3R,5S)-5-(3-(cyclopentyloxy)-4-methoxyphenyl)-2-oxopiperidin-3-yl)methyl)-5,6-dimethylbenzimidazole-1-carboxylate (127 mg, 34%) and tert-butyl 2-(((3S,5S)-5-(3-(cyclopentyloxy)-4-methoxyphenyl)-2-oxopiperidin-3-yl)methyl)-5,6-dimethylbenzimidazole-1-carboxylate (179 mg, 48%) after column separation (EtOAc). Tert-butyl 2-(((3R,5S)-5-(3-(cyclopentyloxy)-4-methoxyphenyl)-2-oxopiperidin-3-yl)methyl)-5,6-dimethyl-benzimidazole-1-carboxylate was then treated with TFA (5 mL) in dichloromethane (20 mL) at 0° C. for 1 hour and then at ambient temperature for 2 hr. The solvents were removed by rotary evaporation, and the residue was purified by column (EtOAc/MeOH, 95/5) to give compound (3R,5S)-5-(3-(cyclopentyloxy)-4-methoxyphenyl)-3-((5,6-dimethyl-benzimidazol-2-yl)methyl)piperidin-2-one (103 mg, quantitative, MW 447.58). (3S,5S)-5-(3-(cyclopentyloxy)-4-methoxyphenyl)-3-((5,6-dimethyl-benzimidazol-2-yl)methyl)piperidin-2-one (158 mg, quantitative, MW 447.58) was obtained in a similar manner.

B. In a similar manner utilizing the appropriately substituted starting material and intermediate compound of formula III, the following compounds were made:
(3S ,5S)-5-(3-cyclopentyloxy-4-methoxyphenyl)-3-(5,6-dichloro-2-benzimidazolylmethyl)piperidin-2-one, MW 488.41;
(3R,5S)-5-(3-cyclopentyloxy-4-methoxyphenyl)-3-(5,6-dichloro-2-benzimidazolylmethyl)piperidin-2-one, MW 488.41;
(3S,5S)-3-(2-benzimidazolylmethyl)-5-(3-cyclopentyloxy-4-methoxyphenyl)piperidin-2-one, MW 419.52;
(3R,5S)-3-(2-benzimidazolylmethyl)-5-(3-cyclopentyloxy-4-methoxyphenyl)piperidin-2-one, MW 419.52;
(3S,5S)-5-(3-cyclopentyloxy-4-methoxyphenyl)-3-(1-methyl-2-benzimidazolylmethyl)piperidin-2-one, MW 433.55; and
(3R,5S)-5-(3-cyclopentyloxy-4-methoxyphenyl)-3-(1-methyl-2-benzimidazolylmethyl)piperidin-2-one, MW 433.55.

### Example 3

A. To a solution of compound (S)-tert-butyl 5-(3-(cyclopentyloxy)-4-methoxyphenyl)-2-oxopiperidine-1-carboxylate (550 mg, 1.41 mmol) in dry THF (7.8 mL) under argon was slowly added 0.71M LDA [2.37 mL, 1.69 mmol, prepared from n-BuLi (2.00 mL, 2.5 M solution in hexane, 5.00 mmol) and diisopropylamine (0.77 mL, 5.49 mmol) in THF (4.23 mL)] at −78° C. The mixture was stirred at −78° C. for one hour, and then DMPU (0.7 mL, 5.79 mmol) was added to the above mixture via syringe. The mixture was stirred for additional 15 minutes.

B. 3-(bromomethyl)pyridine hydrobromide (534 mg, 2.12 mmol) was dissolved in water (1 mL) and toluene (2 mL) was added. The mixture was cooled in an ice/water bath and 1M NaOH solution (2.22 mL, 2.22 mmol) was added dropwise with stirring. After 15 minutes, the layers were separated and aqueous layer was extracted with toluene (1 mL). The combined organic layer was washed with brine, dried over MgSO₄, filtered and the filtrate was added dropwise to the final solution of Paragraph A above at −78° C. The resulting mixture was stirred at −78° C. for 30 minutes, and then was gradually allowed to warm to −40° C. over one hour and stirred for additional 2.5 hours at −40° C. Water was added and the resulting mixture was extracted with diethyl ether (3×). The combined organic layer was washed with water (3×), brine (2×), dried over anhydrous K₂CO₃. After a few minutes 3-mercaptopropionic acid (122 μL, 1.41 mmol) was added and the heterogeneous mixture was stirred at ambient temperature for overnight. Water was added, the layers were separated and the organic portion was washed with saturated NaHCO₃ (2×), brine, dried over MgSO₄, filtered, and concentrated. The crude product was purified with Flashmaster (25% to 50% EtOAc in hexanes) to give (5S)-tert-butyl 5-(3-(cyclopentyloxy)-4-methoxyphenyl)-2-oxo-3-(pyridin-3-ylmethyl)piperidine-1-carboxylate (205 mg, 30%).

C. Trifluoroacetic acid (3 mL) was added to a solution of (5S)-tert-butyl 5-(3-(cyclopentyloxy)-4-methoxyphenyl)-2-oxo-3-(pyridin-3-ylmethyl)piperidine-1-carboxylate (203 mg, 0.42 mmol) in CH₂Cl₂ (3 mL) at 0° C. The mixture was stirred at ambient temperature for two hours and then was concentrated by rotary evaporation. The residue was diluted with dichloromethane, washed with saturated NaHCO₃, brine, dried over MgSO₄, filtered, and concentrated. The residue was purified by preparative TLC (EtOAc/MeOH, 20/1) to afford (3R,5S)-5-(3-(cyclopentyloxy)-4-methoxyphenyl)-3-(pyridin-3-ylmethyl)piperidin-2-one (82 mg, 51%), MW 380.48, and (3S,5S)-5-(3-(cyclopentyloxy)-4-meth-oxyphenyl)-3-(pyridin-3-ylmethyl)piperidin-2-one (20 mg, 12%), MW 380.48.

D. In a similar manner utilizing the appropriately substituted starting material and intermediate compound of formula III, the following compounds were made:
(3R,5S)-5-(3-cyclopentyloxy-4-methoxyphenyl)-3-(6-methyl-2-pyridinylmethyl)piperidin-2-one;
(3R,5S)-5-(3-cyclopentyloxy-4-methoxyphenyl)-3-(6-methyl-2-pyridinyl methyl)piperidin-2-one hydrochloride, MW 430.97;
(3S,5S)-5-(3-cyclopentyloxy-4-methoxyphenyl)-3-(6-methyl-2-pyridinylmethyl)piperidin-2-one, MW 394.51;
(3R,5S)-5-(3-cyclopentyloxy-4-methoxyphenyl)-3-(2-pyridinylmethyl)piperidin-2-one, MW 380.48;
(3S,5S)-5-(3-cyclopentyloxy-4-methoxyphenyl)-3-(2-pyridinylmethyl)piperidin-2-one, MW 380.48;
(3R,5S)-5-(3-cyclopentyloxy-4-methoxyphenyl)-3-(4-pyridinylmethyl)piperidin-2-one, MW 380.48; and
(3S,5S)-5-(3-cyclopentyloxy-4-methoxyphenyl)-3-(4-pyridinylmethyl)piperidin-2-one, MW 380.48.

### Example 4

A. To a solution of compound (S)-tert-butyl 5-(3-(cyclopentyloxy)-4-methoxyphenyl)-2-oxopiperidine-1-carboxylate (1.00 g, 2.57 mmol) in dry THF (14.0 mL) under argon was slowly added 0.71M LDA [4.31 mL, 3.08 mmol, prepared from n-BuLi (2.00 mL, 2.5 M solution in hexane, 5.00 mmol) and diisopropylamine (0.77 mL, 5.49 mmol) in THF (4.23 mL)] at −78° C. The mixture was stirred at −78° C. for one hour, and then DMPU (0.47 mL, 3.85 mmol) was added to the above mixture via syringe. After 30 minutes, tert-butyl bromoacetate (0.46 mL, 3.08 mmol) was added. The resulting mixture was gradually allowed to warm to −40° C. over one hour and stirred for an additional hour at −40° C. The excess base was quenched with aqueous saturated NH₄Cl, and the resulting solution was extracted with EtOAc (4×). The combined organic layer was washed with brine, dried over MgSO₄, filtered and the filtrate was evaporated to dryness. The residue was purified by column (hexanes/EtOAc, 8/2) to give (5S)-tert-butyl 3-(2-tert-butoxy-2-oxoethyl)-5-(3-(cyclopentyloxy)-4-methoxy-phenyl)-2-oxopiperidine-1-carboxylate, as an isomeric mixture, (1.05 g, 81%) as a white solid.

B. Trifluoroacetic acid (3.5 mL) was added to a solution of (5S)-tert-butyl 3-(2-tert-butoxy-2-oxoethyl)-5-(3-(cyclopentyloxy)-4-methoxyphenyl)-2-xopiperidine-1-carboxylate (382 mg, 0.76 mmol) and 1,3-dimethoxybenzene (0.14 mL, 1.04 mmol) in CH₂Cl₂ (3.5 mL) at 0° C. The mixture was stirred at 0° C. for three hours and at ambient temperature for an additional hour, and then was concentrated by rotary evaporation. The residue was purified by radial chromatography (0% to 5% methanol in CH₂Cl₂) to afford 2-((5S)-5-(3-(cyclopentyloxy)-4-methoxyphenyl)-2-oxopiperidin-3-yl)acetic acid, as an isomeric mixture, (226 mg, 86%) as a white solid.

C. A mixture of 2-((5S)-5-(3-(cyclopentyloxy)-4-methoxyphenyl)-2-oxopiperidin-3-yl)acetic acid (218 mg, 0.63 mmol), 2-aminoimidazole sulfate (83 mg, 0.63 mmol), BOP (278 mg, 0.63 mmol) and N,N-diisopropylethylamine (0.33 mL, 1.89 mmol) in dry DMF (5 mL) was stirred at 60° C. under argon for six hours. The mixture was diluted with water and the resulting solution was extracted with EtOAc (4×). The combined organic layer was washed with brine, dried over MgSO₄, filtered and the filtrate was evaporated to dryness. The residue was purified by radial chromatography (0% to 5% methanol in CH₂Cl₂) to give 2-[(5S)-5-(3-cyclopentyloxy-4-methoxyphenyl)-2-oxo-3-piperidinyl]-N-(2-imidazolyl)acetamide as a mixture of diastereomers (119 mg, 46%) as a white solid.

D. 2-[(5S)-5-(3-cyclopentyloxy-4-methoxyphenyl)-2-oxo-3-piperidinyl]-N-(2-imidazolyl)acetamide was further purified by reverse phase HPLC (C-18, 80% to 10% water (with 0.1% trifluoroacetic acid) in acetonitrile) to give 2-[(3R,5S)-5-(3-cyclopentyloxy-4-methoxyphenyl)-2-oxo-3-piperidinyl]-N-(2-imidazolyl)acetamide trifluoroacetic acid salt, MW 526.51 (99 mg), 2-[(3S,5S)-5-(3-cyclopentyloxy-4-methoxyphenyl)-2-oxo-3-piperidinyl]-N-(2-imidazolyl)-acetamide trifluoroacetic acid salt, MW 526.51 (10 mg), and a mixture of the two diastereomers (39 mg) as white solids. The free base of these compound are also prepared, thereby providing 2-[(3R,5S)-5-(3-cyclopentyloxy-4-methoxyphenyl)-2-oxo-3-piperidinyl]-N-(2-imidazolyl)acetamide and 2-[(3S,5S)-5-(3-cyclopentyl-oxy-4-methoxyphenyl)-2-oxo-3-piperidinyl]-N-(2-imidazolyl)acetamide.

E. In a similar manner utilizing the appropriately substituted starting material, the following compounds were made:
2-[(5S)-5-(3-cyclopentyloxy-4-methoxyphenyl)-2-oxo-3-piperidinyl]-N-(3-pyridinyl)acetamide, MW 423.51; 2-[(5S)-5-(3-cyclopentyloxy-4-methoxyphenyl)-2-oxo-3-piperidinyl]-N-(4-pyridinyl)acetamide, MW 423.51;

2-((5S)-5-(3-(cyclopentyloxy)-4-methoxyphenyl)-2-oxopiperidin-3-yl)-N-(4-fluorophenyl)acetamide, MW 440.51; 2-((5S)-5-(3-(cyclopentyloxy)-4-methoxyphenyl)-2-oxopiperidin-3-yl)-N-phenylacetamide, MW 422.52;
N-(4-(1H-imidazol-1-yl)phenyl)-2-((5S)-5-(3-(cyclopentyloxy)-4-methoxyphenyl)-2-oxopiperidin-3-yl)acetamide, MW 488.58;
2-((5S)-5-(3-(cyclopentyloxy)-4-methoxyphenyl)-2-oxopiperidin-3-yl)-N-phenylacetamide, MW 422.52; and
2-((5S)-5-(3-(cyclopentyloxy)-4-methoxyphenyl)-2-oxopiperidin-3-yl)-N-(quinolin-6-yl)acetamide, MW 473.57.

### Example 5

In a similar manner as described above in the foregoing Synthetic Preparations and Synthetic Examples or in a similar manner as the methods described in U.S. Pat. No. 6,770,658 and U.S. Pat. No. 6,458,829, and utilizing the appropriately substituted electrophile and/or starting material and methods known to one skilled in the art; the following compounds of formula (I) are/were prepared:
(5S)-3-(2-(4H-1,2,4-triazol-4-ylamino)ethyl)-5-(3-(cyclopentyloxy)-4-methoxyphenyl)piperidin-2-one, MW 399.49;
(3R,5S)-5-(3-cyclopentyloxy-4-methoxyphenyl)-3-(4-methyl-2-benzimidazolylaminomethyl)piperidin-2-one;
(3S,5S)-5-(3-cyclopentyloxy-4-methoxyphenyl)-3-(4-methyl-2-benzimidazolylaminomethyl)piperidin-2-one;
(S,E)-3-benzylidene-5-(3-(cyclopentyloxy)-4-methoxyphenyl)piperidin-2-one, MW 377.48;
(S,E)-5-(3-(cyclopentyloxy)-4-methoxyphenyl)-3-(4-fluorobenzylidene)piperidin-2-one, MW 395.47;
(S,E)-5-(3-(cyclopentyloxy)-4-methoxyphenyl)-3-(2,4-difluorobenzylidene)piperidin-2-one, MW 413.46;
(S,E)-5-(3-(cyclopentyloxy)-4-methoxyphenyl)-3-(3-fluorobenzylidene)piperidin-2-one, MW 395.47;
(S,E)-5-(3-(cyclopentyloxy)-4-methoxyphenyl)-3-(3-phenoxybenzylidene)piperidin-2-one, MW 469.57;
(S,E)-5-(3-(cyclopentyloxy)-4-methoxyphenyl)-3-((6-methylpyridin-2-yl)methylene)piperidin-2-one, MW 392.49;
(S,E)-3-(4-(1H-imidazol-1-yl)benzylidene)-5-(3-(cyclopentyloxy)-4-methoxyphenyl)piperidin-2-one, MW 443.54;
(S,E)-5-(3-(cyclopentyloxy)-4-methoxyphenyl)-3-(3-methoxybenzylidene)piperidin-2-one, MW 407.50;
(S,E)-5-(3-(cyclopentyloxy)-4-methoxyphenyl)-3-(3-(trifluoromethoxy)benzylidene)piperidin-2-one, MW 461.47;
(S,E)-5-(3-(cyclopentyloxy)-4-methoxyphenyl)-3-(4-(diethylamino)benzylidene)piperidin-2-one, MW 448.60;
(S,E)-5-(3-(cyclopentyloxy)-4-methoxyphenyl)-3-(2-methoxybenzylidene)piperidin-2-one, MW 407.50;
(S,E)-5-(3-(cyclopentyloxy)-4-methoxyphenyl)-3-(4-(diethoxymethyl)benzylidene)piperidin-2-one, MW 479.61;
(S,E)-5-(3-(cyclopentyloxy)-4-methoxyphenyl)-3-(4-(3-(dimethylamino)propoxy)-benzylidene)piperidin-2-one, MW 478.62;
(S,E)-5-(3-(cyclopentyloxy)-4-methoxyphenyl)-3-(3,4-dimethoxybenzylidene)piperidin-2-one, MW 437.53;
(S,E)-5-(3-(cyclopentyloxy)-4-methoxyphenyl)-3-(2,5-difluorobenzylidene)piperidin-2-one, MW 413.46;
(S,E)-5-(3-(cyclopentyloxy)-4-methoxyphenyl)-3-(2-methylbenzylidene)piperidin-2-one, MW 391.50;
(S,E)-3-(2-chlorobenzylidene)-5-(3-(cyclopentyloxy)-4-methoxyphenyl)piperidin-2-1-one, MW 411.92;
(S,E)-5-(3-(cyclopentyloxy)-4-methoxyphenyl)-3-(2-nitrobenzylidene)piperidin-2-one, MW 422.47;
(S,E)-5-(3-(cyclopentyloxy)-4-methoxyphenyl)-3-(3-methylbenzylidene)piperidin-2-one, MW 391.50;
(S,E)-5-(3-(cyclopentyloxy)-4-methoxyphenyl)-3-(3-(trifluoromethyl)benzylidene)piperidin-2-one, MW 445.47;
(S,E)-3-(3-chlorobenzylidene)-5-(3-(cyclopentyloxy)-4-methoxyphenyl)piperidin-2-one, MW 411.92;
(S,E)-3-(benzo[d][1,3]dioxol-4-ylmethylene)-5-(3-(cyclopentyloxy)-4-methoxyphenyl)piperidin-2-one, MW 421.49;
(S,E)-3-(benzo[d][1,3]dioxol-5-ylmethylene)-5-(3-(cyclopentyloxy)-4-methoxyphenyl)piperidin-2-one, MW 421.49;
(S,E)-3-(2-chloro-5-(trifluoromethyl)benzylidene)-5-(3-(cyclopentyloxy)-4-methoxyphenyl)piperidin-2-one, MW 479.92;
(S,E)-5-(3-(cyclopentyloxy)-4-methoxyphenyl)-3-(3,5-dichlorobenzylidene)piperidin-2-one, MW 446.37;
(S,E)-5-(3-(cyclopentyloxy)-4-methoxyphenyl)-3-(furan-3-ylmethylene)piperidin-2-one, MW 367.44;
(S,E)-5-(3-(cyclopentyloxy)-4-methoxyphenyl)-3-(pyridin-2-ylmethylene)piperidin-2-one, MW 378.46;
(S,E)-5-(3-(cyclopentyloxy)-4-methoxyphenyl)-3-(pyridin-3-ylmethylene)piperidin-2-one, MW 378.46;
(S,E)-5-(3-(cyclopentyloxy)-4-methoxyphenyl)-3-(pyridin-4-ylmethylene)piperidin-2-one, MW 378.46;
(S,E)-3-(3-(4-chlorophenoxy)benzylidene)-5-(3-(cyclopentyloxy)-4-methoxyphenyl)piperidin-2-one, MW 504.02;
(S,E)-5-(3-(cyclopentyloxy)-4-methoxyphenyl)-3-(2,3-difluorobenzylidene)piperidin-2-one, MW 413.46;
(S,E)-3-(4-chlorobenzylidene)-5-(3-(cyclopentyloxy)-4-methoxyphenyl)piperidin-2-one, MW 411.92;
(S,E)-5-(3-(cyclopentyloxy)-4-methoxyphenyl)-3-(4-fluoro-2-(trifluoromethyl)benzylidene)piperidin-2-one, MW 463.46;
(S,E)-5-(3-(cyclopentyloxy)-4-methoxyphenyl)-3-(3-fluoro-4-methoxybenzylidene)piperidin-2-one, MW 425.49;
(S,E)-3-(3,5-bis(trifluoromethyl)benzylidene)-5-(3-(cyclopentyloxy)-4-methoxyphenyl)piperidin-2-one, MW 513.47;
(S,E)-5-(3-(cyclopentyloxy)-4-methoxyphenyl)-3-(3,4,5-trimethoxybenzylidene)piperidin-2-one, MW 467.55;
(S,E)-5-(3-(cyclopentyloxy)-4-methoxyphenyl)-3-(4-phenoxybenzylidene)piperidin-2-one, MW 469.57;
(S,E)-5-(3-(cyclopentyloxy)-4-methoxyphenyl)-3-(2,6-dimethoxybenzylidene)-piperidin-2-one, MW 437.53;
(S,E)-5-(3-(cyclopentyloxy)-4-methoxyphenyl)-3-(2-fluorobenzylidene)piperidin-2-one, MW 395.47;
(S,E)-5-(3-(cyclopentyloxy)-4-methoxyphenyl)-3-(4-(trifluoromethoxy)benzylidene)piperidin-2-one, MW 461.47;
(S,E)-5-(3-(cyclopentyloxy)-4-methoxyphenyl)-3-(4-isopropylbenzylidene)piperidin-2-one, MW 419.56;
(S,E)-3-(4-butoxybenzylidene)-5-(3-(cyclopentyloxy)-4-methoxyphenyl)piperidin-2-one, MW 449.58;
(5S)-3-allyl-5-(3-(cyclopentyloxy)-4-methoxyphenyl)piperidin-2-one, MW 329.43;
(5S)-5-(3-(cyclopentyloxy)-4-methoxyphenyl)-3-(2-(phenylamino)ethyl)piperidin-2-one, MW 408.53;
(3R,5S)-5-(3-(cyclopentyloxy)-4-methoxyphenyl)-3-(2-(phenylamino)ethyl)piperidin-2-one, MW 408.53;
(S)-5-(3-(heptyloxy)-4-methoxyphenyl)piperidin-2-one, MW 319.44;
(S)-5-(6-methoxybiphenyl-3-yl)piperidin-2-one, MW 281.35;
4-(2-((5S)-5-(3-(cyclopentyloxy)-4-methoxyphenyl)-2-oxopiperidin-3-yl)ethylamino)benzonitrile, MW 433.54;
(5S)-3-(2-(4H-1,2,4-triazol-4-ylamino)ethyl)-5-(3-(cyclopentyloxy)-4-methoxyphenyl)piperidin-2-one, MW 399.49;
(5S)-5-(3-(cyclopentyloxy)-4-methoxyphenyl)-3-(2-oxo-2-(4-(pyrrolidin-1-yl)piperidin-1-yl)ethyl)piperidin-2-one, MW 483.65;
(3S,5S)-5-(3-(cyclopentyloxy)-4-methoxyphenyl)-3-(2-(4-fluorophenylamino)ethyl)piperidin-2-one, MW 426.53;
(S)-5-(3-ethoxy-4-methoxyphenyl)piperidin-2-one, MW 249.30;
(S)-5-(3-isopropoxy-4-methoxyphenyl)piperidin-2-one, MW 263.33;
2-((5S)-5-(3-(cyclopentyloxy)-4-methoxyphenyl)-2-oxopiperidin-3-yl)-N-(1H-imidazol-2-yl)acetamide , MW 412.48;
(5S)-5-(3-(cyclopentyloxy)-4-methoxyphenyl)-3-(2,3-dihydroxypropyl)piperidin-2-one, MW 363.45;
2-((5S)-5-(3-(cyclopentyloxy)-4-methoxyphenyl)-2-oxopiperidin-3-yl)acetic acid, MW 347.41;
(5S)-5-(3-(cyclopentyloxy)-4-methoxyphenyl)-3-methylpiperidin-2-one, MW 303.4;
2-((5S)-5-(3-(cyclopentyloxy)-4-methoxyphenyl)-2-oxopiperidin-3-yl)acetaldehyde, MW 331.41;
(3R,5S)-5-(3-(cyclopentyloxy)-4-methoxyphenyl)-3-(2-(4-fluorophenylamino)ethyl)piperidin-2-one, MW 426.53;
(S)-3,3-diallyl-5-(3-(cyclopentyloxy)-4-methoxyphenyl)piperidin-2-one, MW 369.50;
(3R,5S)-3-(benzo[d][1,3]dioxol-5-ylmethyl)-5-(3-(cyclopentyloxy)-4-methoxyphenyl)piperidin-2-one, MW 423.50;
(3S,5S)-3-(benzo[d][1,3]dioxol-5-ylmethyl)-5-(3-(cyclopentyloxy)-4-methoxyphenyl)piperidin-2-one, MW 423.50;
(3R,5S)-5-(3-(cyclopentyloxy)-4-methoxyphenyl)-3-((tetrahydrofuran-3-yl)methyl)piperidin-2-one, MW 373.49;
2-(2-methoxy-5-((3S,5S)-5-(3-methylbenzyl)-6-oxopiperidin-3-yl)phenoxy)acetamide, MW 382.45;
(3S,5S)-5-(3-(cyclopentyloxy)-4-methoxyphenyl)-3-((phenylamino)methyl)piperidin-2-one, MW 394.51;
(3S,5S)-5-(3-(cyclopentyloxy)-4-methoxyphenyl)-3-((4-isopropylphenylamino)methyl)piperidin-2-one, MW 436.59;
(3S,5S)-3-((4-chlorophenylamino)methyl)-5-(3-(cyclopentyloxy)-4-methoxyphenyl)piperidin-2-one, MW 428.95;
(3S,5S)-5-(3-(cyclopentyloxy)-4-methoxyphenyl)-3-((m-tolylamino)methyl)piperidin-2-one, MW 408.53;
(3S,5S)-5-(3-(cyclopentyloxy)-4-methoxyphenyl)-3-((4-fluorophenylamino)methyl)piperidin-2-one, MW 412.50;
(3S,5S)-5-(3-(cyclopentyloxy)-4-methoxyphenyl)-3-((3-fluorophenylamino)methyl)piperidin-2-one, MW 412.50;
(3S,5S)-5-(3-(cyclopentyloxy)-4-methoxyphenyl)-3-((3,5-dichlorophenylamino)methyl)piperidin-2-one, MW 463.40;
(3S,5S)-5-(3-(cyclopentyloxy)-4-methoxyphenyl)-3-((3,4-dimethoxyphenylamino)methyl)piperidin-2-one, MW 454.56;
(3S,5S)-5-(3-(cyclopentyloxy)-4-methoxyphenyl)-3-((3-phenoxyphenylamino)methyl)piperidin-2-one, MW 486.61;
(3S,5S)-5-(3-(cyclopentyloxy)-4-methoxyphenyl)-3-((3,5-dimethoxyphenylamino)methyl)piperidin-2-one, MW 454.56;
(3S,5S)-5-(3-(cyclopentyloxy)-4-methoxyphenyl)-3-((4-methoxyphenylamino)methyl)piperidin-2-one, MW 424.53;
(3S,5S)-3-((benzo[d][1,3]dioxol-5-ylamino)methyl)-5-(3-(cyclopentyloxy)-4-methoxyphenyl)piperidin-2-one, MW 438.52;
(3S,5S)-3-((4-tert-butylphenylamino)methyl)-5-(3-(cyclopentyloxy)-4-methoxyphenyl)piperidin-2-one, MW 450.62;
(3S,5S)-5-(3-(cyclopentyloxy)-4-methoxyphenyl)-3-((3,4-difluorophenylamino)methyl)piperidin-2-one, MW 430.49;
(3S,5S)-5-(3-(cyclopentyloxy)-4-methoxyphenyl)-3-((pyridin-4-ylamino)methyl)piperidin-2-one, MW 395.5;
(3S,5S)-5-(3-(cyclopentyloxy)-4-methoxyphenyl)-3-((p-tolylamino)methyl)piperidin-2-one, MW 408.53;
(3S,5S)-5-(3-(cyclopentyloxy)-4-methoxyphenyl)-3-((o-tolylamino)methyl)piperidin-2-one, MW 408.53;
(3S,5S)-5-(3-(cyclopentyloxy)-4-methoxyphenyl)-3-((2-fluorophenylamino)methyl)piperidin-2-one, MW 412.50;
(3S,5S)-5-(3-(cyclopentyloxy)-4-methoxyphenyl)-3-((2,6-dimethylphenylamino)methyl)piperidin-2-one, MW 422.56;
(3S,5S)-5-(3-(cyclopentyloxy)-4-methoxyphenyl)-3-((3-ethoxyphenylamino)methyl)piperidin-2-one, MW 438.56;
(3S,5S)-5-(3-(cyclopentyloxy)-4-methoxyphenyl)-3-((4-ethoxyphenylamino)methyl)piperidin-2-one, MW 438.56;
(3S,5S)-5-(3-(cyclopentyloxy)-4-methoxyphenyl)-3-((3-nitrophenylamino)methyl)-piperidin-2-one, MW 439.51;
(S)-1-benzyl-5-(3-(cyclopentyloxy)-4-methoxyphenyl)piperidin-2-one, MW 379.49;
(S)-5-(3-(cyclopentyloxy)-4-methoxyphenyl)-3-methylenepiperidin-2-one, MW 301.39;
(S)-5-(3-(cyclopentyloxy)-4-methoxyphenyl)-3,3-bis(hydroxymethyl)piperidin-2-one, MW 349.42;
(5S)-5-(3-(cyclopentyloxy)-4-methoxyphenyl)-3-(hydroxymethyl)piperidin-2-one, MW 319.4;
(3S,5S)-3-((benzyl(phenyl)amino)methyl)-5-(3-(cyclopentyloxy)-4-methoxyphenyl)piperidin-2-one, MW 484.63;
(S)-5-(3-(cyclopentyloxy)-4-hydroxyphenyl)piperidin-2-one, MW 275.34;
(S)-5-(3-(cyclopentyloxy)-4-propoxyphenyl)piperidin-2-one, MW 317.42;
(S)-5-(3-(cyclopentyloxy)-4-(2-hydroxyethoxy)phenyl)piperidin-2-one, MW 319.4;
(S)-5-(3-(cyclopentyloxy)-4-(3-hydroxypropoxy)phenyl)piperidin-2-one, MW 333.42;
(S)-ethyl 4-(2-(cyclopentyloxy)-4-(6-oxopiperidin-3-yl)phenoxy)butanoate, MW 389.49;
(S)-4-(2-(cyclopentyloxy)-4-(6-oxopiperidin-3-yl)phenoxy)butanoic acid, MW 361.43;
(S)-1-acetyl-5-(3-(cyclopentyloxy)-4-methoxyphenyl)piperidin-2-one, MW 331.41;
(S)-1-benzoyl-5-(3-(cyclopentyloxy)-4-methoxyphenyl)piperidin-2-one, MW 393.48; and
5-(3-(cyclopentyloxy)-4-methoxyphenyl)-3-(3-methylbenzoyl)pyridin-2(1H)-one, MW 403.47.

### Example 6

Compounds of the examples inhibit disease induction in the models described herein at doses of less than 20 mg/kg. Compounds of the examples were tested in a biological test described herein, and were found to exhibit 50% inhibition of PDE4 at a concentration of 20 μM or below: For example, the following representative compounds of the examples exhibited the following IC₅₀ values when tested in Biological Example 1, Method A:
(S)-5-(6-methoxybiphenyl-3-yl)piperidin-2-one: 146 nM
(S)-1-benzyl-5-(3-(cyclopentyloxy)-4-methoxyphenyl)piperidin-2-one: 430 nM
(S)-1-benzoyl-5-(3-(cyclopentyloxy)-4-methoxyphenyl)piperidin-2-one: 187 nM
(5S)-3-allyl-5-(3-(cyclopentyloxy)-4-methoxyphenyl)piperidin-2-one: 290 nM
(S)-3,3-diallyl-5-(3-(cyclopentyloxy)-4-methoxyphenyl)piperidin-2-one: 651 nM
(5S)-5-(3-(cyclopentyloxy)-4-methoxyphenyl)-3-(2-(phenylamino)ethyl)piperidin-2-one: 215 nM
(5S)-5-(3-(cyclopentyloxy)-4-methoxyphenyl)-3-(2,3-dihydroxypropyl)piperidin-2-one: 446 nM
2-((5S)-5-(3-(cyclopentyloxy)-4-methoxyphenyl)-2-oxopiperidin-3-yl)acetic acid: 1185 nM
(3S,5S)-5-(3-(cyclopentyloxy)-4-methoxyphenyl)-3-(3-pyridinylmethyl)piperidin-2-one: 489 nM
(3R,5S)-5-(3-cyclopentyloxy-4-methoxyphenyl)-3-(5-nitro-2-benzoxazolylmethyl)piperidin-2-one: 653 nM
(3R,5S)-5-(3-cyclopentyloxy-4-methoxyphenyl)-3-(5-methoxy-2-benzofurylmethyl)piperidin-2-one: 373 nM
(S,E)-5-(3-(cyclopentyloxy)-4-methoxyphenyl)-3-(3-methylbenzylidene)piperidin-2-one: 389 nM
(3R,5S)-5-(3-(cyclopentyloxy)-4-methoxyphenyl)-3-((tetrahydrofuran-3-yl)methyl)piperidin-2-one: 1491 nM
2-((5S)-5-(3-(cyclopentyloxy)-4-methoxyphenyl)-2-oxopiperidin-3-yl)-N-(pyridin-3-yl)acetamide: 456 nM

### Biological Example 1

### In Vitro Inhibition of PDE4 Phosphodiesterases

PDE4 U937 cytoplasmic extracts were prepared by a modified procedure of the assay described in MacKenzie, S. J. and Houslay, M. D., “Action of rolipram on specific PDE4 cAMP phosphodiesterase isoforms and on the phosphorylation of cAMP-response-element-binding protein (CREB) and p38 mitogen-activated protein (MAP) kinase in U937 monocytic cells”, Biochem J. (2000), 347(Pt 2):571-8, by lysis of U937 cells (ATCC: Catalogue No. CRL-159) in M-PER Lysis buffer (Pierce) containing 10% protease inhibitor cocktail (Sigma). The cell lysates were then centrifuged at 30,000 rpm for 15 minutes at 4° C. The supernatants were aliquoted and stored at −80° C. PDE4 has been shown to be the predominant cyclic nucleotide phosphodieterase activity in U937 cells.

An alternative source of PDE4 enzymes was from recombinant human PDE4 obtained from baculovirus-SF9 cells expression system. cDNA containing PDE4D1 was cloned into a baculovirus vector, insect cells (SF9) were then infected and cells cultured to express the PDE4 protein. The cells were lysed and used directly in assay or partially purified using standard procedures. The process can be used for other PDE4 and PDE enzymes.

Compounds of the invention were evaluated for inhibitory activity against PDE4 enzymes by the following assay Method A or B.

### Method A:

PDE4 assay based on modified procedure of Phosphodiesterase [³H]cAMP SPA Enzyme Assay (Amersham Biosciences, code TRKQ 7090). In this assay, PDE4 enzymes converts [³H]cAMP to [³H]5′-AMP. The assay is quenched by the addition of SPA yttrium silicate beads which preferentially bind linear nucleotides over cyclic nucleotides in the presence of zinc sulphate. The amount of [³H]5′-AMP formed is proportional to the PDE4 activity, hence PDE4 inhibitors would decrease the amount of [³H]5′-AMP formed.

Reactions were performed in duplicate by the addition of 10 μl PDE4 enzyme (U937 lysate or recombinant hPDE4) to 20 μL of assay mix and 20 μL of test compounds in Isoplates (Wallac) for 30 minutes, at 37° C. The final assay mixture contained: 50 mM Tris (pH 7.5), 8.3 mM MgCl₂, 1.7 mM EGTA and [³H]cAMP (0.025 μCi) (Amersham). Assay was terminated by addition 25 μL SPA beads. The plate was sealed, shaken for 1 minute and then allowed to settle 30 minutes and the cpm determined using a Wallac Micobeta.

### Method B:

PDE4 assay based on modified procedure of Thompson and Appleman (Biochemistry (1971); 10; 311-316). In this assay, PDE4 enzymes converts [³H]cAMP to [³H]5′-AMP. The [³H]5′-AMP is then converted to [³H]adenosine and phosphate by nucleotidase. The amount of [³H]adenosine formed is proportional to the PDE4 activity, hence PDE4 inhibitors would decrease the amount of [³H]adenosine formed.

supplemented lysis buffer. Supplemented lysis buffer consisted of 20 mM Tris, 1 mM EDTA, 1 mM DTT, 0.25 M sucrose, 1 mM benzamidine, 1 μg/mL Leupeptin, 1 μM Pepstatin, and 0.1 mM PMSF, pH 7.5. The platelet lysates were centrifuged at 70,000 g for 30 minutes at 4° C. The supernatants were aliquoted and stored at −80° C.

Reactions were performed in duplicate by the addition of 10 μL PDE3 lysate to 20 μL of assay mix and 20 μL of test compounds in Isoplates (Wallac) for 30 minutes, at 37° C. The final assay mixture contained: 50 mM Tris (pH 7.5), 8.3 mM MgCl₂, 1.7 mM EGTA, 0.1% BSA, and [³H]cAMP (0.025 μCi) (Amersham). Assay was terminated by addition 25 μL SPA beads and 4.75 mM IBMX (non-selective PDE inhibitor) to stop the reaction. The plate was sealed, shaken for 1 minute and then allowed to settle 30 minutes and the cpm determined using a Wallac Micobeta.

Compounds of invention were dissolved in 100% DMSO and diluted such that the final DMSO concentration in the assay did not exceed 1% to avoid affecting the PDE3 activity. PDE3 enzyme was added in quantities such that less than 15% of substrate was consumed (linear assay conditions). Test compounds were assayed at 6-8 concentrations of ranging from 0.1 nM to 100 μM and IC₅₀ values determined from the concentration curves by nonlinear regression analysis.

### Biological Example 3

### In Vitro PDE Specificity Inhibition

Compounds of invention were evaluated for inhibition specificity for phosphodiesterases 1-11. Assay procedures are similar to those described above for in vitro PDE4 inhibition with substitution of cAMP with cGMP for PDE's that hydrolyze cGMP preferentially.

### Biological Example 4

### Inhibition of LPS induced TNF-α Release from Human Peripheral Blood Mononuclear Cells

Compounds of the invention were evaluated for inhibitory activity against lipopolysaccharide (LPS) induced TNF-α release from human peripheral blood mononuclear cells (PBMC's). TNF-α is one of the most harmful endogenous pro-inflammatory cytokine. Production of this cytokine has been repeatedly shown to be potently inhibited in the presence of PDE4 inhibitors in vivo and in vitro which are believed to contribute largely to the anti-inflammatory effects of these drugs, at least under acute inflammatory conditions (Draheim, R. et al., “Anti-Inflammatory Potential of the Selective Phosphodiesterase 4 Inhibitor N-(3,5-Dichloro-pyrid-4-yl)-[1-(4-fluorobenzyl)-5-hydroxy-indole-3-yl]-glyoxylic Acid Amide (AWD 12-281), in Human Cell Preparations”, The Journal of Pharmacology and Experimental Therapeutics (2004), Vol. 308, No. 2: 555-563; and Billah, M. M. et al., “Pharmacology of N-(3,5-Dichloro-1-oxido-4-pyridinyl)-8-methoxy-2-(trifluoromethyl)-5-quinoline Carboxamide (SCH 351591), a Novel, Orally Active Phosphodiesterase 4 inhibitor”, The Journal of Experimental Therapeutics (2002), Vol. 302, No. 1:127-137). The most potent TNF-α producing cell populations belong to the monocyte/macrophage lineage.

There are number of publications showing inhibition of TNF-α release by known PDE4 inhibitors in whole blood as well as isolated PBMC's (Schindler, R. et al., “Correlations and Interactions in the Production of Interleukin-6 (IL-6), IL-1, and Tumor Necrosis Factor (TNF) in Human Blood Mononuclear Cells: IL-6 Suppresses IL-1 and TNF”, Blood (1990), Vol. 75, No. 1: 40-47). Studies with PDE4B knock-out mice revealed that this PDE4 subtype was essential for LPS-induced generation of TNF-α. Therefore, testing the compounds of the invention in this assay served as a convenient cellular screening. The assay validated the ability of compounds of invention to enter the cells and will show some specificity towards desired activity against PDE4B.

### Method

PBMC's were purchased from AllCells, LLC and were prepared according to the manufacturer protocol. Briefly, the frozen vial of PBMC's (AlICells, LLC Catalogue No. PB003F) was removed from cryostorage, thawed quickly in 37° C. water bath and transferred to 50 mL tube containing 300 μg of DNAse. The vial was rinsed with 1 mL of supplemented RPMI 1640 medium pre-warmed in incubator (5% CO₂, 37° C.). Supplemented RPMI 1640 contained 10% FBS, 2 mM L-Glutamine, Penicillin 50 Units/Streptomycin 50 μg/mL and 10 mM. HEPES. The volume was brought to 20 mL with supplemented medium slowly. Cells were centrifuged twice for 15 minutes at 2400 rpm at ambient temperature. PBMC's were counted, diluted to approximately 0.7×10⁶/mL and 150 μL aliquots were dispensed to each well of a tissue culture treated 96-well plate for a final cell density of 1×10⁵/well. As defined by supplier, such preparation contained 12% of monocytes (CD14+). Viability of the cells in each experiment exceeded 90%. Plates were placed in incubator (5% CO₂, 37° C.) for 0.5-1 h to allow monocytes to attach.

Compounds of invention were diluted in V-bottom 96-well plate. Test compounds were first diluted in 100% DMSO, followed by dilutions in enriched RPMI medium to give a final assay DMSO concentration of 0.03%. Plates containing PBMC's were removed from incubator and 40 μL of test compounds were added and the PBMC's pre-incubated with the test compounds for 1 h.

After 1 h pre-incubation with the tested compounds, 10 μL of LPS was added and the plates incubated (5% CO₂, 37° C.) for 18 hours. Final LPS concentration in the assay was 1 ng/mL. Plates were then centrifuged at 2200 rpm (−786 g) for 10 minutes at ambient temperature. 50 μL cell culture supernatants were carefully removed and frozen immediately at −80° C.

Human TNF-α content was determined by ELISA. Cell supernatants were diluted 15 times with appropriate ELISA diluent. Results were obtained either with BD Human TNF-α ELISA (BD) or BioSource ELISA (BioSource). In general, the ELISA assay procedure involved coating Immulon 4 HBX strips or plates with capture antibody (anti-human TNF), washing with PBST (PBS with 0.05% Tween-20) and blocking with 1% BSA or 10% FBS. After another washing, 2 hour incubation with the diluted cell supernatants or standard (recombinant human TNF) was done. Subsequent washing was followed by incubation with detection antibody (biotinylated anti-human TNF) and enzyme reagent (streptavidinhorseradish peroxidase conjugate). After final PBST washing, enzyme substrate (tetramethylbenzidine/hydrogen peroxide) was added. Reaction was stopped by addition of stop solution (2 N sulphuric acid). Read out was obtained by absorbance measurement at 450 nm (reference filter 650 nm) with Multiscan Spectrum plate reader.

Test compounds were assayed at 6-8 concentrations of ranging from 1 nM to 10 μM and IC₅₀ values determined from the concentration curves by nonlinear regression analysis. The inhibition of LPS induced TNF-α release from human PBMC's thereof served as a convenient cellular assay to evaluate PDE4 inhibitors.

### Biological Example 5

### Potentiation of Forskolin-Induced cAMP Response Element Luciferase Activity in Human 0937 Monocytic Cells

In order to demonstrate the ability of compounds of the present invention to elevate cAMP in intact cells, transfection of cells with a plasmid construct containing a cAMP response element (CRE) in a promoter driving the expression of a luciferase reporter gene (Stratagene; Path Detect™: Catalogue No. 219076) was used to allow sensitive monitoring of intracellular cAMP levels through detection of light output in a luminometer. Pharmacological treatment of transfected cells with a compound providing a combination of PDE inhibitor and adenylyl cyclase agonist (receptor or intracellular activator) resulted in elevated intracellular cAMP levels detectable from increased light output. PDE4 has been shown to be the predominant cyclic nucleotide phosphodieterase activity in U937 cells, and therefore this cell type transfected with the CRE-luciferase construct served as a convenient cellular screening assay for compounds with PDE4 inhibitory activity. Compounds of the present invention were thereby shown to provide potentiated luciferase expression in U937 cells treated with the adenylyl cyclase activator forskolin.

U937 cells were maintained in RPMI medium containing 10% FCS and 2 mM glutamate. U937 cells were transiently transfected as described in Biotechniques (1994), Vol. 17(6):1058. Briefly, cells were grown in medium containing serum to a density of 5×10⁶ cells/mL and then resuspended in media containing serum at a density of approximately 1×10⁷ cells/mL. 400 μL of cells were transferred into the electroporation cuvette containing 10 μg of the reporter vector (pCRE-luc) in a volume of 40 μL H₂O. Reporter vector DNA was prepared from DH5α E. coli using the DNA endonuclease free kit (Qiagen) as per manufacturers instructions. U937 cells were electroporated at ambient temperature using a BIORAD electroporator. Capacitance was set to 1050 μμF and voltage was 280V. The time constant was noted after each electroporation. Cells were then diluted in 4 mL of media and serum and 200 ppt of cells were plated per well. Cells were allowed to recover for 16-18 hours. Cells were then treated with a test compound or vehicle in the presence or absence of 10 μM forskolin for 4 hours at 37° C.

The luciferase assay was performed as per manufacturer's instructions (Tropix). Briefly, cells were centrifuged for 4 minutes at 1200 rpm and media supernatant was removed. Cell pellets were lysed in 15 μL Lysis buffer (Tropix). Luciferase assay was performed using 10 ppt of cell lysate with 10 μL of buffer A and 25 μL buffer B. Luciferase activity was obtained using a luminometer with a 5 second delay followed by a read time of 10 seconds.

None of the test compounds in the absence of stimuli induced significant luciferase activity indicating a low basal adenylyl cyclase activity in these cells. This result demonstrated that the compounds tested were capable of elevating cAMP levels in a cell line predominantly expressing PDE4 consistent with the observations in the enzymatic assays.

### Biological Example 6

### Effect of Compounds on Ear Edema in a TH1 Mouse Model of Chemical Hapten Delayed Type Hypersensitivity

Delayed type hypersensitivity models are T cell dependent responses. The type of chemical hapten used can bias the T cell response towards a predominantly TH1 or TH2 polarization. Oxazolone and di-nitro-chloro-benzene (DNCB) induce a TH1 dominant immune response.

Mice are sensitized on day 0 by epicutaneous application of 100 μL 3% oxazolone solution in 95% ethanol on the shaved abdomen. This procedure is repeated on day 1. Six days after sensitization (i.e., on day 5), mice are challenged by topically painting 25 μL 0.8% oxazolone dissolved in 95% ethanol on both sides of the right ears and 25 μL of 95% ethanol on the left ears. On day 6 (24 hours after challenge), mice are sacrificed, both ears are removed and a standard disc of tissue is harvested immediately from each ear using a cork borer. Care is taken to sample the tissues from the same ear area. The weight of the ear disc tissues is immediately measured. Test compounds are administered orally at a dose of 5 mg/kg once daily for 7 days (from day 0 to day 6) with the last dose 2 hours prior to sacrifice.

Alternatively, mice are sensitized on day 0 by epicutaneous application of 50 μL 1% di-nitrochlorobenzene (DNCB) solution in 4: ratio of acetone:olive oil on the shaved abdomen. This procedure is repeated on day 5. Starting eleven days after the initial sensitization, mice are challenged 3 times (on days 10, 11, and 12) by topically painting 25 μL 0.5% DNCB dissolved in a 4:1 ratio of acetone:olive oil on both sides of the right ears and 25 μL of vehicle on the left ears. Twenty-four hours after challenge, mice are sacrificed as described above. Test compounds are administered orally at a dose of 10 mg/kg once daily for 5 days (from day 8 to day 12) with the last dose 2 hours prior to challenge.

Ear edema is expressed as increase in ear weight, and calculated by subtracting the left ear weight (challenged with vehicle) from that of right ear (challenged with chemical hapten). The percentage inhibition of the ear edema by drugs is calculated using following equation: 100−((drug edema/mean control edema)*100).

Compounds of the invention may inhibit oxazolone and DNCB induced dermal inflammation at doses of less than 20 mg/kg.

### Biological Example 7

### Effect of Compounds on Ear Edema in a TH2 Mouse Model of Delayed Type Hypersensitivity to Fluorescein Isothiocyanate

Mice are sensitized on day 0 by epicutaneous application of 50 μL 0.5% fluorescein isothiocyanate (FITC) solution in 1:1 acetone and dibutyl phthalate on the shaved abdomen. This procedure is repeated on day 7. Fourteen days after sensitization (i.e., on day 13), mice are challenged by topically painting 25 μL 0.5% FITC dissolved in 1:1 acetone and dibutyl phthalate on both sides of the right ears and 25 μL 1:1 acetone and dibutyl phthalate solution on the left ears. On day 14 (24 hours after challenge), mice are sacrificed, both ears are removed and a standard disc of tissue is harvested immediately from each ear using a cork borer. Care is taken to sample the tissues from the same ear area. The weight of the ear disc tissues is immediately measured. Test compounds (5-10 mg/kg) or vehicle is administered orally once daily for 3 days (from day 11 to day 13) 2 hours prior to challenge.

Ear edema is expressed as increase in ear weight, and calculated by subtracting the left ear weight (challenged with vehicle) from that of right ear (challenged with FITC). The percentage inhibition of the ear edema by drugs is calculated using the following equation: 100−((drug edema/mean control edema)*100).

Compounds of the invention may inhibit FITC induced dermal inflammation at doses of less than 20 mg/kg.

### Biological Example 8

### Effect of Compounds on Irritant-Induced Mouse Ear Edema

A number of mice are uniquely identified by placing a mark with an indelible marker on their tail. Mice are dosed orally with 15 mg/kg test compound in 100 μL of 45% β-cyclodextrin in saline. Mice are briefly anaesthesized with 2% halothane, and 2 μg of phorbol 12-myristate 13-acetate (PMA) in 25 μl of acetone is applied to the inner and outer sides of the left ear of the mouse. Acetone is applied to the right ear of the mouse in the same manner to serve as a vehicle control. Control animals receive the same treatment but without any test compound. After 3 hours, mice are sacrificed by cervical dislocation, and a standard sized biopsy is excised from the ears and weighed to the nearest 1/10th of a mg. Data are analyzed by taking the difference of each left ear from the right ear, and then calculating the % inhibition of edema by (((mean Rx/mean irritant))×100)−100.

Compounds of the invention may inhibit PMA induced dermal edema at doses of less than 20 mg/kg.

### Biological Example 9

### Effect of Selected Compounds on Collagen-induced Arthritis (CIA) in Mice

The collagen-induced arthritis (CIA) model in mice is a suitable model for evaluating potential drugs active in human rheumatoid arthritis. It shares many of the molecular, cellular and histopathological changes identified as hallmarks of the human disease; these include (a) pronounced proliferation of cells comprising the joint synovial membrane, (b) formation of an invasive pannus-like tissue, (c) macrophage, granulocyte and lymphocytic infiltration and (d) destruction of bone and cartilage. Like rheumatoid arthritis, animals with CIA exhibit elevated serum levels of immunoglobulin complexes such as rheumatoid factor (RF) and anti-collagen antibodies and inflammatory cytokines in the synovium such as tumour necrosis factor (TNF-α). In addition, involvement of MHC class II-restricted T-helper cell activation/clonal expansion in the synovium has been demonstrated. Radiographs of affected joints often show erosive changes similar to those seen in human RA and the progressive arthritis often results in an RA-like joint deformity and dysfunction. In addition, many compounds which reduce the symptoms of human disease such as anti-TNF biologics, corticosteroids and DMARDS are efficacious in this animal model. The development/progression of disease in the CIA model occurs in both an immune (early) and inflammatory phase thus allowing the assessment of a wide range of drugs with diverse pharmacological modes of action.

Male DBA/1J mice (7-8 weeks of age) are immunized through a subcutaneous injection of 0.1 mL of a collagen-adjuvant emulsion (0.1 mg chick type II collagen in complete Freund's adjuvant) at the base of the tail. Mice are then randomly assigned to treatment or control groups. After three weeks the animals are boosted with a second injection of chick type II collagen emulsified at 1.0 mg/mL in incomplete Freund's adjuvant. This second injection is required for reproducible induction of disease. In control animals, clinical signs of arthritis manifested as erythema and edema of the paws and tarsal/metatarsal joints usually appear within 1-2 weeks following the second immunization. Compounds are evaluated for their ability to delay the onset of or reduce the development of arthritis (prophylactic regime). Compounds are administered twice daily beginning on the day of the second collagen injection. The mice continue to receive test article until the last animal in the vehicle control group reached the seventh day of having established disease (approximately 25 days).

The development of clinical arthritis (disease progression) is monitored daily after the second collagen injection. All four limbs are clinically evaluated by a trained observer unfamiliar (blinded) with the treatment group identity, and scored on a scale of 0-4 for disease severity (redness and swelling) according to the following criteria.

| Score | Condition |
|---|---|
| 0 | Normal |
| 1 | Some joints swollen and red, but not all |
| 2 | All joints swollen |
| 3 | Full inflammation of paw |
| 4 | Maximum inflammation, no further swelling possible |

Inflammation is defined as any redness or swelling (enlargement) of any part of any paw. Established disease was defined as a qualitative score of paw inflammation of 2 or greater, that persists for at least 24 hours. In addition, paw widths for all four limbs were measured by a blinded observer daily using precision, constant tension calipers.

At the end of the study each animal is euthanized by an overdose of halothane anesthesia. Joints both distal to the knee and including the knee are dissected and analyzed by histology. Limb joints are fixed in 10% formalin buffer and decalcified in 10% formic acid for 48 hours, then processed for paraffin embedding. Serial sections (5-7 micrometer thick) are stained with haematoxylin and eosin (H & E). Histopathological alterations of the tarsal and metatarsal joints are graded “blind” by a certified pathologist and a score assigned based on a ranking system. ANOVA and appropriate post-hoc test will be used to determine if arthritis scores from test article treated animals will be significantly lower than those of the vehicle treated animals.

Compounds of the invention may inhibit clinical signs of CIA-iduced arthritis at doses of less than 20 mg/kg.

### Biological Example 10

### Effect of Compounds on Cartilage Degradation in Mice

This model is used to investigate the effect of novel compounds on cartilage degradation induced by the natural inflammatory response created by implantation of a foreign body. Activity in this model may be indicative of activity in arthritis.

Zyphoid sternum cartilage is excised from CO₂ terminated rats, washed in Hibitane, and rinsed in sterile, phosphate buffered saline. A 4 cm diameter disc is removed from the sternum with a # 4 stainless steel leather hole punch, and cut in half. Each half is weighed and wrapped in pre-weighed, moist, sterile cotton before implantation. A piece of cotton wrapped cartilage is implanted subcutaneously into each dorsolateral surface of anaesthetized female CD/1 mice (aged 6-8 weeks) via a 1 cm incision along the dorsal midline (Day 0). Mice are administered test articles by oral administration on days 3 to 17. On day 18, mice are sacrificed, the cotton and cartilage removed, and the cartilage separated from the cotton. Both the cartilage and the cotton are weighed, and differences between pre and post implant weights are calculated. The cotton is rinsed in 1 mL of buffer, and cytospins are prepared and stained for differentiation and enumeration of cell types. In addition, the resuspended lavage fluid is analyzed for absolute cell numbers and cell differentials by the CellDyn 3700SC hematology analyzer (Abbott Laboratories Inc.).

The cartilage is digested overnight in a papain and cysteine hydrochloric acid solution at 65° C. and glucosaminoglycan content remaining in the cartilage is assayed by spectrophotometrically and calculated as % GAG/mg of cartilage degraded (normalized to pre implant cartilage weight).

Compounds of the invention may inhibit cartilage degradation at doses of less than 20 mg/kg.

### Biological Example 11

### Effect of Compounds on LPS-Induced Joint Inflammation in Mice

This model is used to investigate the effect of novel compounds on joint inflammation induced by LPS. Joint inflammation occurs in the joints of patients with rheumatoid arthritis. Activity in this model may be indicative of activity in arthritis.

Balb/C mice will be injected directly into left hind knee joint with 3 ng of LPS (6 μl of stock) using an Hamilton syringe (H80401) adapted to a 30G needle. A 9 mm long spacer made of PE10 tubing will be placed on needle to insure LPS was injected to the same depth for each animal. Care will be taken to ensure no fluid is drawn back after each injection. The same volume of saline (6 μl) will be injected into the right hind knee, as a control, using a separate Hamilton syringe. Eighteen hours after challenge, animals will be anesthetized with 5% isoflurane and euthanized by cardiac puncture. The hind limbs will be dissected free from attached muscles and removed. The synovial cavity of each leg will be exposed by pulling the patellar tendon towards the distal end of the leg, and will be washed with 3 ml of ice-cold EDTA (10 mM)-PBS buffer. The washout solution will be centrifuged at 1200 rpm for 3 min. Supernatant will be removed and the cell pellet will be resuspended in 0.5 ml cold PBS/EDTA. Total cell counts and differentials in the synovial washout will be counted using a Cell Dyne hematology analyzer and cytospin preparations.

Data will be plotted as mean±SEM. One-way ANOVA followed by student Newman-Kuels all-pairwise or Dunnett's post-hoc test will be used for comparison of multiple means. P<0.05 will be considered statistically significant. Compounds of the invention may inhibit joint inflammation at doses of less than 20 mg/kg.

### Biological Example 12

### Effect of Selected Compounds on LPS -induced Acute Lung Inflammation in Rat

Rats are administered drug (1-20 mg/kg) or vehicle orally once (0-24 hours) prior to challenge. Rats are challenged with either saline or LPS dissolved in saline (2 mg/kg) via intra-tracheal installation. Animals are sacrificed via intra-peritoneal sodium pentobarbitol overdose 3 hours post challenge, and the lungs lavaged with 14 mL of phosphate buffered saline (PBS). The lung lavage fluid is centrifuged at 300 g for 3 min, and the supernatant removed. The pellet is resuspended in 1-3 mL of PBS at 4° C. depending on pellet size and numbers of total leukocytes. A volume of the final cell suspension, containing approximately 240,000 cells, is added to an appropriate volume of PBS at 4° C. to give a final volume of 220 μL and a final concentration of 1×10⁶ cells/mL (final Cytospin suspension). A 100 μL sample (100,000 cells) is loaded onto a cytospin centrifuge and spun for 4 min at 55 g. Two slides are prepared per lavage sample, and are fixed and stained in DifQuik. In addition, the resuspended lavage fluid is analyzed for absolute cell numbers and cell differentials by the CellDyn 3700SC hematology analyzer (Abbott Laboratories Inc.). This model could be adapted to assess effect of selected compounds in a mouse model of LPS-induced lung inflammation.

Compounds of the invention may inhibit LPS induced lung inflammation at doses of less than 20 mg/kg.

### Biological Example 13

### Effect of Selected Compounds on Allergen-Induced Lung Inflammation in the Rat

The ability of a compound to inhibit the allergen-induced accumulation of inflammatory cells such as eosinophils and neutrophils in the lavage fluid obtained from sensitized animals is indicative of that compound's anti-asthma activity. In particular, this model system is useful in the evaluation of the effects of a test compound in the treatment of the late phase response of asthma, when lung inflammation and the second phase of bronchoconstriction is apparent, and in allergy, especially where it affects the respiratory system. The test is conducted as follows.

Male Brown Norway rats are sensitized to ovalbumin by single intraperitoneal injection of 1 mg ovalbumin adsorbed to 100 mg Al(OH)₃ (alum) in 1 mL sterile saline (saline control rats receive only sterile saline) on day 1, and allowed to sensitize until day 21. Test compounds are given orally q.d. for three days prior to challenge (days 19, 20, 21), and one day post challenge (day 22), with the third dose given 2 hours before challenge, and the fourth day dose given 24 hours after challenge (volume=300 μl/dose). Rats are challenged with 5% ovalbumin in saline generated using a Devillbis nebulizer for 5 min on day 21.

Forty-eight hours after challenge, animals are sacrificed with an overdose of intraperitoneally-delivered sodium pentobarbitol and the lungs are lavaged, with cold 2×7 mL phosphate buffered saline. The recovered lavage fluid is placed on ice. The bronchoalveolar lavage fluid is centrifuged and the supernatant removed. The pellet is resuspended in phosphate buffered saline at 4° C. Cytospins are prepared and stained for differentiation and enumeration of cell types. This model could be adapted to assess effect of selected compounds in a mouse model of allergen-induced lung inflammation.

Compounds of the invention may inhibit allergen induced lung inflammation at doses of less than 20 mg/kg.

### Biological Example 14

### Effect of Selected Compounds on Allergen-Induced Airwayhyper-responsiveness in the Mouse

The Buxco murine airway hyper-responsiveness (AHR) model has been well characterized by numerous investigators, and mimics the severe airway constriction in response to aerosol challenges that sensitized animals exhibit compared to unsensitized animals. The Buxco system uses a technique called whole body plethysmography, in which breathing-induced changes in chamber pressure are quantified using the correlation between increased airway resistance and increased expiratory time/breathing pause to calculate the degree of airway constriction (Penh). Following allergen sensitization and inhalation challenge of the airway, the Penh will increase compared to sham sensitized, sham challenged animals. Thus the effectiveness of a potential anti-inflammatory agent can be determined by examining its impact on ovalbumin induced AHR.

Female Balb/c mice are sensitized on day 1 and 14 by i.p. injection of 100 μL sterile saline containing 20 μg ovalbumin and 2.25 mg Al(OH)₃. Sham sensitized mice receive 100 μL sterile saline alone. Test compounds (5 mg/kg) are administered by oral gavage avage on five consecutive days, two days before challenge (days 26 and 27) and on the three days of ovalbumin challenge (days 28, 29 and 30, 2 hours before challenge). Mice are challenged with aerosolized ovalbumin (5% in saline) for 20 min on days 28, 29 and 30. On day 31, mice are placed in the whole body plethysmography chambers of the Buxco system and airway reactivity to aerosolized PBS and methacholine (MCh; 0.78, 1.56, 3.125, 6.25, 12.5, 25 mg/mL) challenge is measured as Penh.

Compounds of the invention may inhibit allergen induced airway hyper-reactivity at doses of less than 20 mg/kg.

### Biological Example 15

### Effect of Selected Compounds on DSS Induced Colitis in Mice

Inflammatory bowel disease (IBD) is an umbrella term for presently incurable, chronic, fluctuating inflammatory diseases of the gastrointestinal tract including Crohn's disease and ulcerative colitis.

The dextran sodium sulphate (DSS) induced colitis model in mice has been shown to mimic the nature of the human disease, produce lesions that are histopathologically similar to those in humans with similar clinical pathology to that of human disease including, necrosis, formation of ulcers, granulocytic infiltration, edema of the bowel, diarrhea and adhesions with many drugs used to treat human IBD showing activity in the DSS model.

Colitis is induced by oral administration of DSS (in drinking water) (2.5-3% DSS) to groups of 8 female, CD-1 or C57BL/6 mice weighing 15-25 g. Body weight, clinical signs, diarrhea, colonic myeloperoxidase levels and histopathology for ulceration are viable and relevant endpoints.

Compounds of the invention may reduce the effects of DSS on the above endpoints in rats at doses of less than 20 mg/kg.

### Biological Example 16

### Effect of Selected Compounds on TNBS Induced Colitis in Rats

The trinitrobenzenesulfonic acid (TNBS) induced colitis model in rat (Morris et al., Gastroenterology 96:795-803, 1989; Kim, H.-S, and Berstad, A., Scandinavian Journal of Gastroenterology 27:529-537, 1992; Ward, Lancet ii:903-905, 1977; and Shorter et al., Am. J. Dig. Dis. 17:1024-1032, 1972)) has been shown to mimic the relapsing/remitting nature of the human disease, produce lesions that are histopathologically similar to those in humans with similar clinical pathology to that of human disease including, necrosis, formation of ulcers, granulocytic infiltration, edema of the bowel, diarrhea and adhesions with many drugs used to treat human IBD showing activity in the TNBS model.

Colitis is induced by intracolonic instillation of the hapten TNBS (60 mg/mL) in 0.5 mL of 50% ethanol to groups of 8 male, Wistar rats weighing 175-225 g. Body weight, diarrhea, colonic myeloperoxidase levels and histopathology for ulceration are viable and relevant endpoints.

Compounds of the invention may reduce the effects of TNBS on the above endpoints in rats at doses of less than 20 mg/kg.

### Biological Example 17

### Effect of Selected Compounds on Object Recognition Model of Learning and Memory in the Mouse

Several behavioral models of learning and memory exist. They revolve around the ability of animals to recall a previous exposure to an object, environment or stimulus. The behavior of an animal that recalls such an exposure is usually different from the behavior of an animal that is naïve to that exposure. One example of such a model is an object recognition model using a mouse which har been engineered to be heterozygous for CREB binding protein (CBP). A similar heterozygous trait occurs in humans with Rubinstein Taybi Syndrome. One characteristic of this syndrome and these mice is the ability to form short-term memory, but no long term memory. Also CBP function is reduced, but not abolished. Increasing cAMP levels with a PDE4 inhibitor is hypothesized to enhance long term memory generation via and enhancement of CBP activity.

Object recognition in mice can be easily assessed since mice are naturally inquisitive of new objects. Mice are firstly exposed to two identical but novel objects and allowed to investigate them for 15 minutes. The objects are removed and twenty four hours later the mice are re-introduced to one such object and a second completely novel object. If the mouse has long term memory capacity it will preferentially ignore the training object and spend more time investigating the novel object. If there is no long term memory the mice will spend equal time investigating the two objects. The CBP heterozygous mice behave in this maner and have no recall of the training object.

Compounds of the invention may improve object recognition and hence learning and memory in such a mouse object recognition model at doses of less than 20 mg/kg.

### Biological Example 18

### Effect of Selected Compounds on Fear Conditioning Model of Learning and Memory in the Mouse

Several behavioral models of learning and memory exist. They revolve around the ability of animals to recall a previous exposure to an object, environment or stimulus. The behavior of an animal that recalls such an exposure is usually different from the behavior of an animal that is naïve to that exposure. One example of such a model is a mouse fear conditioning model. Mice are trained to recognize an environment that will supply them with an unwanted stimulus, such as a mild foot shock, following an audable tone. In mice that have formed long term memory of the training environment exposure to the same environment and audible tone three days later produces a fear “freeze” response.

Compounds of the invention may improve long term memory consolidation and enhance the “freeze” response of normal or aged mice in this fear conditioning model at doses of less than 20 mg/kg.

### Biological Example 19

### Effect of Selected Compounds on Forced Swim Test for Depression in the Mouse

In humans, depression is commonly characterized by a feeling of helplessness and loss of interests and energy. Clinical depression is hypothesized to result from an imbalance of signals in the brain. Classical anti-depressants, such as the tricyclic antidepressant, desipramine, increase the levels of one of these signals and help to restore signal balance. PDE4 is an enzyme responsible for turning off this signal. Therefore treatment with PDE4 inhibitors is hypothesized to enhance the brain signal as is achieved with the classical antidepressants, albeit via a novel mechanism.

The mouse forced swim test exposes mice to a setting that induces helplessness and frustration, capturing the essence of clinical depression. The forced swim test is one of the most widely used pharmacological method of assessing depression. The test involves placing a mouse in a cylinder of water and then monitoring the time spent swimming versus the time spent floating immobile (helpless time). Immobility indicates a state of despair where the animal has realized that escape is impossible and resigns itself to floating helplessly.

Antidepressant compounds, such as desipramine, that have demonstrated therapeutic effect in humans have also decreased the time of immobility in mice in the forced swim test.

Compounds of the invention may reduce immobility time of mice in the forced swim test at doses of less than 20 mg/kg.

1. A compound of formula (I), 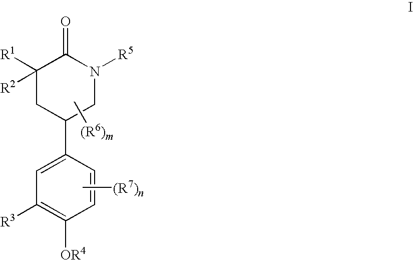
wherein:
m represents 0, 1, 2, 3, 4 or 5;
n represents 0, 1, 2 or 3;
R¹ represents hydrogen, C₁₋₁₂ alkyl, C₂₋₁₂ alkenyl, C₂₋₁₂ alkynyl, -A¹-T^{z}-B¹, -A^{1a}-N(R⁹)R¹⁰, -A^{1b}-OR⁹, -A^{1c}-C(O)R⁹, -A^{1d}-C(O)OR⁹ or -A^{1e}-C(O)N(R⁹)R¹⁰, wherein C₁₋₁₂ alkyl, C₂₋₁₂ alkenyl, and C₂₋₁₂ alkynyl are optionally substituted by one or more substituents selected from X¹;
R² represents hydrogen, —OR⁴, C₁₋₁₂ alkyl, C₂₋₁₂ alkenyl or C₂₋₁₂ alkynyl, which latter three groups are optionally substituted by one or more substituents selected from X¹; or
R¹ and R² together form ═C(R⁹)R¹⁰;
R³ represents hydrogen, —OR⁴, C₁₋₁₂ alkyl, C₂₋₁₂ alkenyl, C₂₋₁₂ alkynyl or -A²-B², wherein C₁₋₁₂ alkyl, C₂₋₁₂ alkenyl, C₂₋₁₂ alkynyl are optionally substituted by one or more substituents selected from X²;
represents hydrogen, —R⁸—OR⁹, —R⁸—C(O)OR⁹, C₁₋₁₂ alkyl, C₂₋₁₂ alkenyl, C₂₋₁₂ alkynyl or -A³-B³, wherein C₁₋₁₂ alkyl, C₂₋₁₂ alkenyl, C₂₋₁₂ alkynyl are optionally substituted by one or more substituents selected from X³;
R⁵ represents hydrogen, -A⁴-B⁴, —C(O)R⁹, —C(O)OR¹⁰, C₁₋₁₂ alkyl, C₂₋₁₂ alkenyl or C₂₋₁₂ alkynyl, which latter three groups are optionally substituted by one or more substituents selected from X⁴;
R⁶ represents halo, —R¹¹—OR⁹, —R¹¹—CN, —R¹¹—NO₂, —R¹¹—C(O)OR⁹, —R¹¹—N(R⁹)R¹⁰, —R¹¹—C(O)N(R⁹)R¹⁰, —R¹¹—N(R^{w3})C(O)R⁹, —R¹¹—N(R^{w3})C(O)N(R⁹)R¹⁰, —R¹¹—N(R^{w3})S(O)ₜR^{9x}, —R¹¹—N(R^{w3})S(O)ₜOR^{9x}, —R¹¹—OC(O)R⁹, —R¹¹—OC(O)N(R)R¹⁰, —R¹¹—OS(O)ₜR^{9x}, —R¹¹—S(O)ₚR⁹, —R¹¹—S(O)ₜN(R^{w3})R⁹, —R¹¹—S(O)ₜOR⁹; —R¹¹—Si(R¹⁶)₃, C₁₋₁₂ alkyl, C₁₋₁₂ alkenyl, C₁₋₁₂ alkynyl, C₃₋₁₅ cycloalkyl or heterocyclyl which latter five groups are optionally substituted by one or more substituents selected from X⁵; or
any two R⁶ groups, or R² and any R⁶ group, may be linked together to form a further ring, which is formed either by the two relevant groups being linked together by a direct bond or C₁₋₅ alkylene;
R⁷ represents halo, —R¹¹—OR⁹, —R¹¹—CN, —R¹¹—NO₂, —R¹¹—C(O)OR⁹, —R¹¹—N(R⁹)R¹⁰, —R¹¹—C(O)N(R⁹)R¹⁰, —R¹¹—N(R^{w3})C(O)R⁹, —R¹¹—N(R^{w3})C(O)N(R⁹)R¹⁰, —R¹¹—N(R^{w3})S(O)ₜR^{9x}, —R¹¹—N(R^{w3})S(O)ₜOR^{9x}, —R¹¹—OC(O)R⁹, —R¹¹—OC(O)N(R)R¹⁰, —R¹¹—OS(O)ₜR^{9x}, —R¹¹—S(O)ₚR⁹, —R¹¹—S(O)ₜN(R^{w3})R⁹, —R¹¹—S(O)ₜOR⁹, —R¹¹—Si(R¹⁶)₃, C₁₋₁₂ alkyl, C₁₋₁₂ alkenyl, C₁₋₁₂ alkynyl, C₃₋₁₅ cycloalkyl or heterocyclyl which latter five groups are optionally substituted by one or more substituents selected from X⁵;
T^{z} represents a direct bond, —N(R^{w1})— or —C(O)N(R^{w2})—;
R^{9x} represents C₁₋₁₂ alkyl, C₂₋₁₂ alkenyl, C₂₋₁₂ alkynyl, -A⁵-O-A⁶ and/or -A⁷-B⁷, wherein C₁₋₁₂ alkyl, C₂₋₁₂ alkenyl C₂₋₁₂ alkynyl are optionally substituted by one or more substituents selected from X⁶;
R⁹, R¹⁰, R^{w1}, R^{w2} and R^{w3} each independently represent hydrogen, C₁₋₁₂ alkyl, C₂₋₁₂ alkenyl, C₂₋₁₂ alkynyl -A⁵-O-A⁶ and/or -A⁷-B⁷, wherein C₁₋₁₂ alkyl, C₂₋₁₂ alkynyl are optionally substituted by one or more substituents selected from X⁶;
or R⁹ and R¹⁰, together with the carbon or nitrogen atom to which they are both attached, may be linked together to form a cycloalkyl or heterocyclyl group both of which are optionally substituted by one or more substituents selected from Z^{1a} or an aryl or heteroaryl group (both of which are optionally substituted by one or more substituents selected from Z^{1b}; and
R¹¹ represents a direct bond or R⁸;
A¹, A^{1a}, A^{1b}, A^{1c}, A^{1d}, A^{1e}, A⁴ and A⁵ each independently represent C₁₋₁₂ alkylene, C₂₋₁₂ alkenylene or C₂₋₁₂ alkynylene, which latter three groups are optionally substituted by one or more substituents selected from X⁷;
A², A³ and A⁷ each independently represent a direct bond, C₁₋₁₂ alkylene, C₂₋₁₂ alkenylene or C₂₋₁₂ alkynylene, which latter three groups are optionally substituted by one or more substituents selected from X⁸;
A⁶ represents C₁₋₁₂ alkyl, C₂₋₁₂ alkenyl or C₂₋₁₂ alkynyl, all of which are optionally substituted by one or more substituents selected from X⁹;
R⁸ represents C₁₋₁₂ alkylene, C₂₋₁₂ alkenylene or C₂₋₁₂ alkynylene, all of which are optionally substituted by one or more substituents selected from X¹⁰;
B¹ represents heteroaryl Optionally substituted by one or more substituents selected from Z^{2a} or a heterocyclyl Optionally substituted by one or more substituents selected from Z^{2b}, wherein when B¹ represents a polycyclic heteroaryl group, then the point of attachment of B¹ to the T^{z} is via a heterocyclyl or heteroaromatic ring of the polycycle;
B², B³ and B⁷ each independently represent aryl Optionally substituted by one or more substituents selected from Y¹), cycloalkyl, which cycloalkyl group is optionally substituted by one or more substituents selected from Z³, heterocyclyl Optionally substituted by one or more substituents selected from Z^{4a} or heteroaryl Optionally substituted by one or more substituents selected from Z^{4b};
B⁴ represents aryl optionally substituted by one or more substituents selected from Y²;
X¹ represents G¹, C₃₋₁₅ cycloalkyl Optionally substituted by one or more T² substituents, heterocyclyl optionally substituted by one or more T³ substituents, heteroaryl optionally substituted by one or more T⁴ substituents, ═O, —Si(R¹⁶)₃, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶, —S(O)ₜOR¹⁶, —S(O)ₚR¹⁶, —S(O)ₜN(R¹⁴)₂, —N(R¹⁴)C(O)N(R¹⁴)₂, —N(R¹⁴)S(O)ₜOR¹⁶, —OC(O)N(R¹⁴)₂ or —OS(O)ₜR^{9x};
X², X³, X⁴, X⁵, X⁶, X⁷, X⁸, X⁹ and X¹⁰ each independently represent G¹, aryl Optionally substituted by one or more T¹ substituents, C₃₋₁₅ cycloalkyl optionally substituted by one or more T² substituents, heterocyclyl Optionally substituted by one or more T³ substituents, heteroaryl Optionally substituted by one or more T⁴ substituents, ═O, —Si(R¹⁶)₃, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶, —S(O)ₜOR¹⁶, —S(O)ₚR¹⁶, —S(O)ₜN(R¹⁴)₂, —N(R¹⁴)C(O)N(R¹⁴)₂, —N(R¹⁴)S(O)ₜOR¹⁶, —OC(O)N(R¹⁴)₂ or —OS(O)ₜR^{9x};
Y¹ and Y² each independently represent -A^{x}-B^{y}, G¹, G², —R¹⁵—OR¹⁷-N(R¹⁴)₂ or —R¹⁵—O—R¹⁷—N(R¹⁴)S(O)ₜR¹⁶;
Z^{1a}, Z^{1b}, Z^{2a}, Z^{2b}, Z³, Z^{4a} and Z^{4b} each independently represent G¹, ═O, ═S, -A^{x}-B^{y} or G²;
G¹ represents C₁₋₁₂ alkyl optionally substituted by one or more substituents selected from T⁵, C₂₋₁₂ alkenyl, C₂₋₁₂ alkynyl, halo, —CN, —NO₂ or ═O, wherein C₂₋₁₂ alkenyl, and C₂₋₁₂ alkynyl are optionally substituted by one or more substituents selected from T⁶;
G² represents -A^{x}-B^{x}, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶, —R¹⁵—S(O)ₜOR¹⁶, —R¹⁵—S(O)ₚR¹⁶ and/or —R¹⁵—S(O)ₜN(R¹⁴)₂;
A^{x} represents a direct bond or O₁₋₁₂ alkylene optionally substituted by one or more halo or ═O substituents;
B^{x} represents aryl or heteroaryl, which groups are optionally substituted by one or more substituents selected from T⁷ and T⁸, respectively;
B^{y} represents cycloalkyl or heterocyclyl, both of which are optionally substituted by one or more substituents selected from halo, C₁₋₆ alkyl, C₂₋₆ alkenyl, C₂₋₆ alkynyl, —OCH₃, —OCHF₂, —OCF₃ or ═O, wherein C₁₋₆ alkyl, C₂₋₆ alkenyl, and C₂₋₆ alkynyl are optionally substituted by one or more halo substituents;
T¹, T⁴, T⁵, T⁶, T⁷ and T⁸ each independently represent halo, C₁₋₆ alkyl, C₂₋₆ alkenyl, C₂₋₆ alkynyl, C₂₋₆ alkynyl —OH, —O—C₁₋₆ alkyl, —OC₂₋₆ alkenyl, —OC₂₋₆ alkynyl, —N(R^{w})₂, —NO₂ and/or —CN; or
T⁵ and T⁶ may alternatively or additionally represent ═O, wherein C₁₋₆ alkyl, C₂₋₆ alkenyl and C₂₋₆ alkynyl are optionally substituted by one or more substituents selected from Q^{x1} and —O—C₁₋₆ alkyl, —OC₂₋₆ alkenyl, and —OC₂₋₆ alkynyl are optionally substituted by one or more substituents selected from Q^{x2};
T² and T³ each independently represent halo, C₁₋₆ alkyl, C₂₋₆ alkenyl, C₂₋₆ alkynyl —OCH₃, —OCHF₂, —OCF₃ or ═O, wherein C₁₋₆ alkyl, C₂₋₆ alkenyl, and C₂₋₆ alkynyl are optionally substituted by halo;
Q^{x1} and Q^{x2} each independently represent halo, —OCH₃, —OCHF₂, —OCF₃, —N(R^{w})₂ or ═O;
R^{w} represents hydrogen, C₁₋₆ alkyl, C₂₋₆ alkenyl, C₂₋₆ alkynyl, which latter three groups are optionally substituted by one or more substituents selected from halo, —OCH₃, —OCHF₂, —OCF₃ or ═O; or
two R^{w} groups, when attached to the same nitrogen atom, may be linked together to form, together with the nitrogen atom to which they are necessarily attached, a 5- or 6-membered ring, optionally containing a further heteroatom and optionally substituted by one or more substituents selected from fluoro, —CH₃ and ═O;
t represents 1 or 2;
p represents 0, 1 or 2;
R¹⁴ represents hydrogen, -A^{x1}-B^{x1}, C₁₋₁₂ alkyl, C₂₋₆ alkenyl or C₂₋₆ alkynyl, which latter three groups are optionally substituted by one or more substituents selected from E¹;
R¹⁵ represents a direct bond, C₁₋₁₂ alkylene or C₂₋₁₂ alkenylene, which latter two groups are optionally substituted by one or more substituents selected from halo, —OCH₃, —OCHF₂, —OCF₃ and ═O;
R¹⁶ represents C₁₋₁₂ alkyl, C₂₋₆ alkenyl, C₂₋₆ alkynyl (which latter three groups are optionally substituted by one or more halo and/or ═O groups) or A^{y1}-B^{y1};
R¹⁷ represents C₁₋₁₂ alkylene or C₂₋₁₂ alkenylene, both of which are optionally substituted by one or more substituents selected from halo and ═O;
A^{x1} and A^{y1} each independently represent a direct bond or C₁₋₁₂ alkylene optionally substituted by one or more halo or ═O groups;
B^{x1} and B^{y1} each independently represent cycloalkyl, heterocyclyl, aryl or heteroaryl, wherein cycloalkyl, heterocyclyl are optionally substituted by one or more substituents selected from halo and ═O and aryl or heteroaryl are optionally substituted by one or more halo atoms;
E¹ represents halo, —CN, —NO₂, ═O, —OR¹⁸, —OC(O)—R¹⁸, —N(R¹⁸)₂, —C(O)R¹⁸, —C(O)OR¹⁸, —C(O)N(R¹⁸)₂, —N(R¹⁸)C(O)OR¹⁹, —N(R¹⁸)C(O)R¹⁹, —N(R¹⁸)S(O)ₜᵢ R¹⁹, —S(O)ₜ₁OR¹⁹, —S(O)ₚ₁R¹⁹, —S(O)ₜ₁N(R¹⁸)₂, —N(R¹⁸)C(O)N(R¹⁸)₂, —N(R¹⁸)S(O)ₜ₁OR^{19x}, —OC(O)N(R¹⁸)₂, —OS(O)ₜ₁R^{19x} and/or —Si(R^{19x})₃;
R¹⁸ and R¹⁹ each independently represents hydrogen, C₁₋₃ alkyl, C₂₋₃ alkenyl or C₂₋₃ alkynyl, which latter three groups are optionally substituted by one or more halo atoms;
R^{19x} represents C₁₋₃ alkyl, C₂₋₃ alkenyl or C₂₋₃ alkynyl, which latter three groups are optionally substituted by one or more halo atoms;
t1 represents 1 or 2;
p1 represents 0, 1 or 2,
or a pharmaceutically acceptable salt, solvate, prodrug or polymorph thereof,
provided that:
(B) when R⁴ represents methyl, R², R³ and R⁵ all represent hydrogen, n represents 0, m represents 1, and R⁶ represents methyl substituted α to the —N(R⁵)— moiety, then R¹ does not represent unsubstituted methyl;
(C) when R² represents hydrogen, m and n both represent O, R⁴ represents methyl, then R¹ does not represent hydrogen when R³ represents —OR⁴ in which wherein R⁴ represents cyclopentyl or methyl and R⁵ represents hydrogen, benzyl or —C(O)OR¹⁰, wherein R¹⁰ represents tert-butyl;
(D) when R² represents hydrogen, m and n both represent O, R³ represents hydrogen, R⁵ represents methyl, then R¹ does not represent hydrogen when R⁴ represents methyl substituted by X³, wherein X³—C(O)N(R¹⁴)₂ and R¹⁴ represents isopropyl.

2. The compound according to claim 1, wherein R¹ represents hydrogen, C₁₋₁₂ alkyl, C₂₋₁₂ alkenyl, C₂₋₁₂ alkynyl, -A¹-T^{z}-B¹, -A^{1a}-N(R⁹)R¹⁶, -A^{1b}-OR⁹, -A^{1c}-C(O)R⁹, -A^{1d}-C(O)OR⁹ or -A^{1e}-C(O)N(R)R¹⁶; wherein C₁₋₁₂ alkyl, C₁₋₁₂ alkenyl, C₁₋₁₂ alkynyl are optionally substituted by one or more substituents selected from halo and OH.

3. The compound according to claim 1, wherein R¹ and R² are not hydrogen.

4. The compound according to claim 1, wherein m and n each independently represent 0, 1, or 2.

5. The compound according to claim 1, wherein R² represents hydrogen, C₁₋₁₂ alkyl or C₁₋₁₂ alkenyl, wherein C₁₋₁₂ alkyl or C₁₋₁₂ alkenyl are optionally substituted by one or more substituents selected from hydroxy or halo.

6. The compound according to claim 1, wherein R³ represents -A²-B², hydrogen or —OR⁴.

7. The compound according to claim 1, wherein each R⁴ represents hydrogen, —R⁸—OR⁹, —R⁸—C(O)OR⁹, C₁₋₁₂ alkyl Optionally substituted by one or more substituents selected from X³—) or -A³-B³.

8. The compound according to claim 1, wherein A² and A³ each independently represent C₁₋₃ alkylene or a direct bond.

9. The compound according to claim 1, wherein B³ represents C₃₋₁₅ cycloalkyl Optionally substituted by one or more substituents selected from Z³— or a 5- to 10-membered heterocyclyl group Optionally substituted by one or more substituents selected from Z^{4a}).

10. The compound according to claim 1, wherein R⁵ represents -A⁴-B⁴, hydrogen or —C(O)R⁹.

11. The compound according to claim 1, wherein R⁶ and R⁷ each independently represent halo, —R¹¹—OR⁹, —R¹¹—CN, —R¹¹—NO₂, —R¹¹—C(O)OR⁹, —R¹¹—N(R⁹)R¹⁰, —R¹¹—C(O)N(R⁹)R¹⁰ or C₁₋₁₂ alkyl optionally substituted by one or more substituents selected from X⁵.

12. The compound according to claim 1, wherein R⁸ represents C₁₋₁₂ alkylene optionally substituted by one or more substituents selected from X¹⁰.

13. The compound according to claim 1, wherein R⁹ and R¹⁰ each independently represent hydrogen, C₁₋₁₂ alkyl, C₁₋₁₂ alkenyl (optionally substituted with X⁶, -A⁵-O-A⁶ or -A⁷-B⁷; or R⁹ and R¹⁰ are linked together to form, together with the nitrogen atom, a 5- or 6-membered heterocyclyl group, which is optionally substituted by one or more substituents selected from halo, —CH₃ and ═O.

14. The compound according to claim 1, wherein A¹, A^{1a}, A^{1b}, A^{1c}, A^{1d}, A^{1e}, A⁴ and A⁵ each independently represent C₁₋₆ alkylene.

15. The compound according to claim 1, wherein A⁶ represents C₁₋₆ alkyl.

16. The compound according to claim 1, wherein A², A³ and A⁷ each independently represent a direct bond or C₁₋₆ alkylene.

17. The compound according to claim 1, wherein X¹, X², X³, X⁴, X⁵, X⁶, X⁷, X⁸, X⁹, X¹⁰, Y¹ and Y² each independently represent G¹.

18. The compound according to claim 1, wherein Z^{1a}, Z^{1b}, Z^{2a}, Z^{2b}, Z³, Z^{4a} and Z^{4b} each independently represent G² or G¹.

19. The compound according to claim 1, wherein G¹ represents halo or C₁₋₆ alkyl optionally substituted by one or more halo atoms.

20. The compound according to claim 1, wherein G² represents —R¹⁵—O—R¹⁴.

21. The compound according to claim 20, wherein R¹⁵ represents a direct bond.

22. The compound according to claim 1, wherein R¹⁴ represents C₁₋₆ alkyl optionally substituted by one or more substituents selected from —C(O)N(R¹⁸)₂ and halo.

23. The compound according to claim 22, wherein R¹⁸ represents hydrogen.

24. The compound according to claim 1, wherein R^{w1} and R^{w2} each independently represent hydrogen or C₁₋₃ alkyl optionally substituted by one or more halo atoms.

25. The compound according to claim 1, wherein when B¹ represents a monocyclic heteroaryl group selected from imidazolyl, triazolyl, pyridyl, thienyl or furanyl.

26. The compound according to claim 1, wherein when B¹ represents a polycyclic heteroaryl group selected from 1,3-dihydroindol-2-one-yl, 2,3-dihydrobenzo[1,4]dioxinyl, benzo[1,4]oxazinyl, pyrrolopyridinyl, imidazopyridyl, thiazolopyridyl, benzoxazolyl, benzimidazolyl, benzofuranyl, indolyl, benzothienyl, benzothiazolyl, benzotriazolyl or oxazolopyridinyl.

27. The compound according to claim 1, wherein:
X¹, X², X³, X⁴, X⁵, X⁶, X⁷, X⁸, X⁹ and X¹⁰ each independently represent G¹, aryl (optionally substituted by one or more T¹ substituents), C₃₋₁₅ cycloalkyl (optionally substituted by one or more T² substituents), heterocyclyl (optionally substituted by one or more T³ substituents), heteroaryl (optionally substituted by one or more T⁴ substituents), ═O, —Si(R¹⁶)₃, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶, —S(O)ₜOR¹⁶, —S(O)ₚR¹⁶, —S(O)ₜN(R¹⁴)₂, —N(R¹⁴)C(O)N(R¹⁴)₂, —N(R¹⁴)S(O)ₜOR¹⁶, —OC(O)N(R¹⁴)₂ and/or —OS(O)ₜR^{9x};
provided that:
(A) when R¹ represents methyl substituted by X¹, R² represents hydrogen, m and n both represent O, R⁴ represents methyl:
(I) when R³ represents —OR⁴ in which R⁴ represents cyclopentyl:
(i) R⁵ represents hydrogen, then X¹ does not represent unsubstituted phenyl, 2-methylphenyl, 3-methylphenyl, 4-methylphenyl, 4-isopropylphenyl, 2-chlorophenyl, 3-chlorophenyl, 3-methoxyphenyl, 3-ethoxyphenyl, 3-propoxyphenyl, 3-butoxyphenyl, 4-butoxyphenyl, 3-pentyloxyphenyl, 3-hexyloxyphenyl, 3-heptyloxyphenyl, 3-phenoxyphenyl, 4-fluorophenyl, 3-benzyloxyphenyl, 3-trifluoromethylphenyl, 4-trifluoromethylphenyl, 4-trifluoromethoxyphenyl, 3-methoxy-4-hydroxyphenyl, 3-methoxy-4-benzyloxyphenyl, 3-(4-chloro-phenoxy)phenyl, 4-phenoxyphenyl, 2-chloro-5-trifluoromethyl-phenyl, or benzodioxol-5-yl (especially when the compound is in the (3R,5R) orientation);
(ii) R⁵ represents —C(O)OR¹⁰, in which R¹⁰ represents tert-butyl, then X¹ does not represent 3-methoxy-4-benzyloxyphenyl;
(iii) R⁵ represents isobutyl or —C(O)R⁹, in which R⁹ represents methyl or unsubstituted phenyl, then X¹ does not represent 3-methylphenyl;
(II) when R³ represents —OR⁴ in which R⁴ represents methyl:
(i) R⁵ represents hydrogen or benzyl, then X¹ does not represent 3-methoxy-4-benzyloxyphenyl or 3-methoxy-4-hydroxyphenyl;
(ii) R⁵ represents —C(O)OR¹⁶, in which R¹⁰ represents tert-butyl, then X¹ does not represent 3-methoxy-4-benzyloxyphenyl;
(III) when R³ represents —OR⁴ in which R⁴ represents isopropyl:
(i) R⁵ represents hydrogen, then X¹ does not represent unsubstituted phenyl, 4-trifluoromethylphenyl or 3-benzyloxyphenyl;
(IV) when R³ represents —OR⁴ in which R⁴ represents ethyl:
(i) R⁵ represents hydrogen, then X¹ does not represent unsubstituted phenyl, 4-fluorophenyl or 3-benzyloxyphenyl;
(B) when R⁴ represents methyl, R², R³ and R⁵ all represent hydrogen, n represents 0, m represents 1, and the R⁶ substituent represents methyl substituted α to the —N(R⁵)— moiety, then R¹ does not represent unsubstituted methyl;
(C) when R² represents hydrogen, m and n both represent O, R⁴ represents methyl, then R¹ does not represent hydrogen when R³ represents —OR⁴ in which R⁴ represents cyclopentyl or methyl and R⁵ represents hydrogen, benzyl or —C(O)OR¹⁰, in which R¹⁰ represents tert-butyl; and
(D) when R² represents hydrogen, m and n both represent 0, R³ represents hydrogen, R⁵ represents methyl, then R¹ does not represent hydrogen when R⁴ represents methyl substituted by X³, in which X³—C(O)N(R¹⁴)₂ and each R¹⁴ represents isopropyl.

28. The compound according to claim 27, wherein when R² represents hydrogen, then R¹ does not represent optionally substituted benzyl.

29. The compound according to claim 28 without provisos (C) and (D), or a pharmaceutically acceptable salt thereof, for use as a pharmaceutical.

30. A pharmaceutical formulation including a compound of formula I, as defined in claim 27, without provisos (C) and (D), or a pharmaceutically acceptable salt thereof, in admixture with a pharmaceutically acceptable adjuvant, diluent, carrier or excipient.

31. The compound according to claim 1 without the provisos, or a pharmaceutically acceptable salt thereof, for use in the treatment of: i) an inflammatory disorder; ii) a disorder in which the modulation of intracellular cyclic adenosine 5′-monophosphate levels within a mammal is desired, which disorder may be an inflammatory disorder; iii) a disorder associated with pathological conditions that are modulated by inhibiting enzymes associated with secondary cellular messengers; iv) transplant rejection in a mammal; v) uncontrolled cellular proliferation; or vi) a disorder associated with the central nervous system.

32. Use of a compound of formula I according to claim 1 without the provisos, or a pharmaceutically acceptable salt thereof, for the manufacture of a medicament for the treatment of a disorder.

33. The Use according to claim 32, wherein the disorder is inflammation, a proliferative disorder or a disease or pathological condition of the central nervous system.

34. The Use according to claim 32, wherein the disorder is ankylosing spondylitis, arthritis, asthma, chronic obstructive pulmonary disease, chronic bronchitis, respiratory distress syndrome, rhinitis, allergic rhinitis, Crohn's disease, nephritis, eczema, atopic dermatitis, urticaria, conjunctivitis, ulcerative colitis, rheumatoid arthritis, osteoarthritis, eosinophilic gastrointestinal disorders, vascular disease and diabetes mellitus fibromyalgia syndrome, gout, inflammations of the brain, emphysema, inflammatory bowel disease, irritable bowel syndrome, ischemia-reperfusion injury juvenile erythematosus pulmonary sarcoidosis, Kawasaki disease, osteoarthritis, pelvic inflammatory disease, psoriatic arthritis (psoriasis), rheumatoid arthritis, psoriasis, tissue/organ transplant, scleroderma, spondyloarthropathies, systemic lupus erythematosus, pulmonary sarcoidosis, ulcerative colitis, cancer, leukemia, a solid tumor, cognitive function, Alzheimer's disease, a learning and memory disorder, cerebrovascular disease, depression, schizophrenia, Parkinson's disease or multiple sclerosis.

35. A method of treatment of a disorder, said method comprising administering a therapeutically effective amount of a compound of claim 1 without the provisos, or a pharmaceutically-acceptable salt thereof, to a patient suffering from, or susceptible to, a disorder selected from i) an inflammatory disorder; ii) a disorder in which the modulation of intracellular cyclic adenosine 5′-monophosphate levels within a mammal is desired, which disorder may be an inflammatory disorder; iii) a disorder associated with pathological conditions that are modulated by inhibiting enzymes associated with secondary cellular messengers; iv) transplant rejection in a mammal; v) uncontrolled cellular proliferation; or vi) a disorder associated with the central nervous system.

36. A combination product comprising:
(A) a compound of formula I as defined in claim 1 without the provisos, or a pharmaceutically-acceptable salt thereof; and
(B) another therapeutic agent that is useful in the treatment of a disorder selected from i) an inflammatory disorder; ii) a disorder in which the modulation of intracellular cyclic adenosine 5′-monophosphate levels within a mammal is desired, which disorder may be an inflammatory disorder; iii) a disorder associated with pathological conditions that are modulated by inhibiting enzymes associated with secondary cellular messengers; iv) transplant resection in a mammal; v) uncontrolled cellular proliferation; or yl) a disorder associated with the central nervous system,
wherein each of components (A) and (B) is formulated in admixture with a pharmaceutically-acceptable adjuvant, diluent, carrier or excipient.

37. The combination product according to claim 36 wherein components (A) and (B) are formulated in a single composition with pharmaceutically-acceptable adjuvant, diluent, carrier or excipient.

38. A kit comprising:
(a) a pharmaceutical formulation including a compound of claim 1 without the provisos, or a pharmaceutically-acceptable salt thereof, in admixture with a pharmaceutically-acceptable adjuvant, diluent, carrier or excipient; and
(b) a pharmaceutical formulation including another therapeutic agent that is useful in the treatment of a disorder selected from i) an inflammatory disorder; ii) a disorder in which the modulation of intracellular cyclic adenosine 5′-monophosphate levels within a mammal is desired, which disorder may be an inflammatory disorder; iii) a disorder associated with pathological conditions that are modulated by inhibiting enzymes associated with secondary cellular messengers; iv) transplant rejection in a mammal; v) uncontrolled cellular proliferation; or vi) a disorder associated with the central nervous system as in admixture with a pharmaceutically-acceptable adjuvant, diluent, carrier or excipient,
which components (a) and (b) are each provided in a form that is suitable for administration in conjunction with the other.

39. A process for the preparation of a compound of the formula I as defined in claim 1 or claim 27, said process comprising:
(i) for compounds of formula I in which R¹ represents -A¹-T^{z}-B¹, reaction of a compound of formula II, 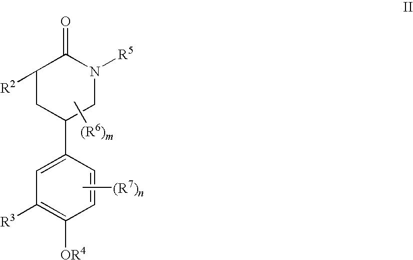
or protected derivatives thereof, wherein R², R³, R⁴, R⁵, R⁶, R⁷, m and n are as defined in claim 1, with a compound of formula III,
L¹-A¹-T^{z}-B¹ III
wherein L¹ represents a suitable leaving group, and T^{z}, A¹ and B¹ are as defined in claim 1;
(ii) for compounds of formula I in which R¹ represents -A¹-T^{z}-B¹ and T^{z} represents —N(R^{w1})—, or R¹ represents -A^{1a}-N(R⁹)R¹⁰ or -A^{1b}-OR⁹, reaction of a compound of formula IV, 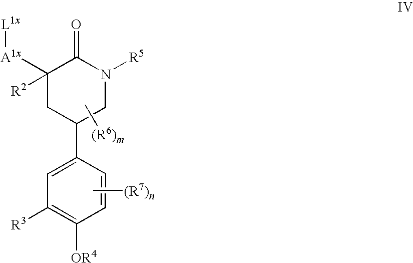
wherein L^{1x} represents a suitable leaving group, A^{1x} represents A¹, A^{1a} or A^{1b} for the preparation of compounds of formula I in which R¹ represents -A¹-N(R^{w1})-B¹, -A^{1a}-N(R⁹)R¹⁰ or -A^{1b}-OR⁹, and R², R³, R⁴, R⁵, R⁶, R⁷, m and n are as defined in claim 1, with a compound of formula V,
H—Z^{a} V
wherein Z^{a} represents —N(R⁹)R¹⁰ or —OR⁹ (for the preparation of compounds of formula I in which R′ represents -A¹-N(R^{w1})-B¹, -A^{1a}-N(R⁹)R¹⁰ or -A^{1b}-OR⁹, respectively), and R^{w1}, B¹, R⁹ and R¹⁰ are as defined in claim 1;
(iii) for compounds of formula I in which R¹ represents -A¹-T^{z}-B¹ and T^{z} represents —C(O)—N(R^{w2})—, or R¹ represents -A^{1e}-C(O)N(R⁹)R¹⁰, reaction of a compound of formula VI, 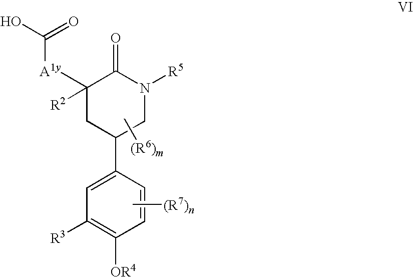
or a protected derivative thereof, wherein A^{1y} represents A¹ or A^{1e} for the preparation of compounds of formula I in which R¹ represents -A¹-C(O)N(R^{w2})—B¹ or -A^{1e}-C(O)N(R⁹)R¹⁰, and R², R³, R⁴, R⁵, R⁶, R⁷, R⁹, R¹⁰, R^{w2}, B¹, A¹, A^{1e}, m and n are as defined in claim 1, with a compound of formula VII,
H—Z^{b} VII
wherein Z^{b} represents —N(R^{w2})—B¹ or —N(R⁹)R¹⁰ (for the preparation of compounds of formula I in which R¹ represents -A¹-C(O)—N(R^{w2})—B¹ or -A^{1e}-C(O)N(R⁹)R¹⁰, and R^{w2}, B¹, R⁹ and R¹⁰ are as defined in claim 1;
(iv) for compounds of formula I in which R¹ represents C₁₋₁₂ alkyl, C₂₋₁₂ alkenyl, C₂₋₁₂ alkynyl, -A^{1a}-N(R⁹)R¹⁰, -A^{1b}-OR⁹, -A^{1c}-C(O)R⁹, -A^{1d}-C(O)OR⁹ or -A^{1e}-C(O)N(R⁹)R¹⁰, and R⁹ and R¹⁰ do not represent hydrogen, reaction of a compound of formula II as defined above, with a compound of formula VIIA,
L^{1b}-Z^{c} VIIA
wherein L^{1b} represents a suitable leaving group, Z^{c} represents C₁₋₁₂ alkyl, C₂₋₁₂ alkenyl, C₂₋₁₂ alkynyl, -A^{1a}-N(R⁹)R¹⁰, -A^{1b}-OR⁹, -A^{1c}-C(O)R⁹, -A^{1d}-C(O)OR⁹ or -A^{1e}-C(O)N(R⁹)R¹⁰, but in which R⁹ and R¹⁰ represent a group other than hydrogen;
(v) for compounds of formula I in which R¹ and R² together form ═C(R⁹)R¹⁰, dehydration of a compound of formula VIIB, 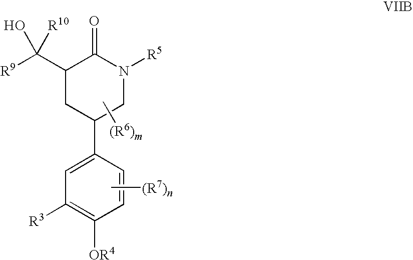
wherein R³, R⁴, R⁵, R⁶, R⁷, R⁹, R¹⁰, m and n are as defined in claim 1;
(vi) intramolecular cyclisation of a compound of formula VIIC, 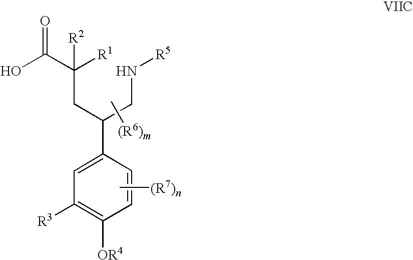
or a protected derivative thereof, wherein R¹, R², R³, R⁴, R⁵, R⁶, R⁷, m and n are as defined in claim 1;
(vii) compounds of formula I in which R¹ represents hydrogen and R² represents —OR⁴ in which R⁴ represents hydrogen may be prepared by reaction of a corresponding compound of formula I in which R² represents hydrogen, with a base, followed by quenching with oxygen or a suitable equivalent thereof;
(viii) compounds of formula I in which R¹ represents hydrogen and R² represents —OR⁴ in which R⁴ represents hydrogen may be prepared by reaction of a compound of formula VIID, 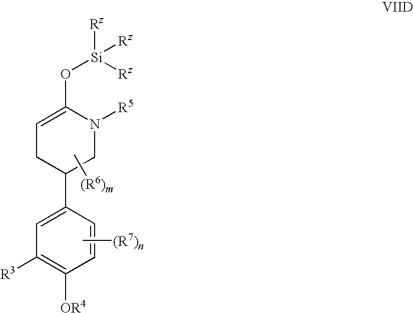
or a protected derivative thereof, wherein each R^{z} independently represents C₁₋₆ alkyl, and R³, R⁴, R⁵, R⁶, R⁷, m and n are as defined in claim 1, under double bond epoxidation reaction conditions;
(ix) compounds of formula I in which R¹ represents hydrogen and R² represents —OR⁴ in which R⁴ represents hydrogen may be prepared by reaction of a compound of formula VIIE, 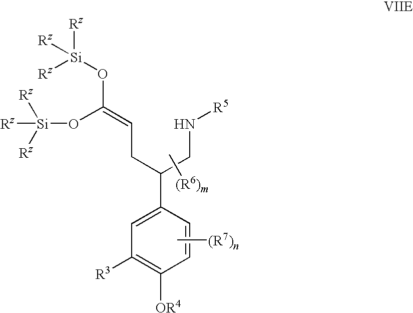
or a protected derivative thereof, wherein R^{z} is as defined above, and R³, R⁴, R⁵, R⁶, R⁷, m and n are as defined in claim 1, in the presence of a suitable oxidising agent;
(x) compounds of formula I may be prepared by reaction of a compound of formula VIII, 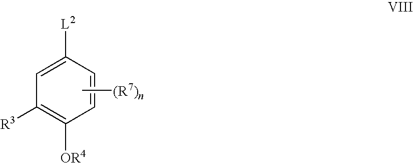
wherein L² represents a suitable leaving group, and R³, R⁴, R⁷ and n are as defined in claim 1, with a compound of formula IX, 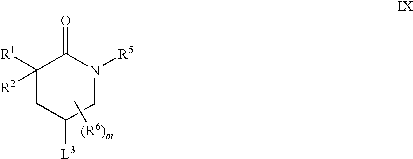
or a tautomer thereof or derivative thereof, wherein L³ represents a suitable leaving group, and R², R⁵, R⁶, L³ and m are as defined in claim 1;
(xi) for compounds of formula I in which R² represents hydrogen, and there is a maximum of two R⁶ substituents present at the 4- and/or 6-position, reduction of a compound of formula IXA, 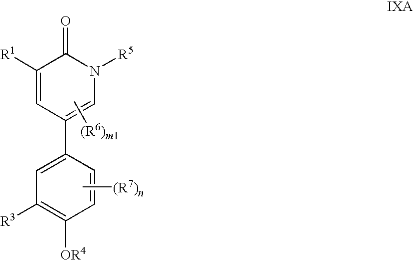
or a tautomer or protected derivative thereof, wherein m1 represents 0, 1 or 2, and R¹, R³, R⁴, R⁵, R⁶, R⁷ and n are as defined in claim 1;
(xii) for compounds of formula I in which R³ represents —OR⁴ in which R⁴ is other than hydrogen, or for compounds of formula I in which R⁴ is other than hydrogen, reaction of a corresponding compound of formula I in which R³ represents —OH or, R⁴ represents hydrogen, with a compound of formula IXB,
R^{4a}-L^{2x} IXB
wherein R^{4a} represents —R⁸—OR⁹, —R⁸—C(O)OR⁹, C₁₋₁₂ alkyl, C₂₋₁₂ alkenyl, C₂₋₁₂ alkyneyl (which latter three groups are optionally substituted by one or more substituents selected from X³) or -A³-B³, L^{2x} represents a suitable leaving group, and R⁸, R⁹, X³, A³ and B³ are as defined in claim 1;
(xiii) for compounds of formula I in which R³ represents —OR⁴ in which R⁴ is other than hydrogen, or for compounds of formula I in which R⁴ is other than hydrogen, reaction of a compound of formula IXC, 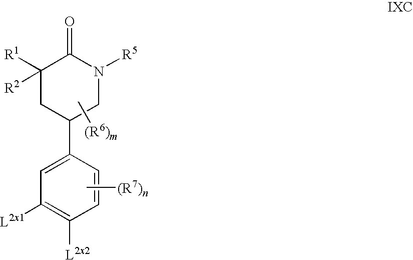
wherein L^{2x1} represents L^{2x} or R³, L^{2x2} represents L^{2x} or —OR⁴, provided that at least one of R^{2x1} and R^{2x2} represents L^{2x}, L^{2x} is as defined above, and R¹, R², R⁵, R⁶, R⁷, m and n are as defined in claim 1, with a compound of formula IXD,
R⁴—OH IXD
wherein R⁴ is as defined in claim 1.

40. A process for the preparation of a pharmaceutical formulation as defined in claim 30, which process comprises bringing into association a compound of formula I, without provisos (C) and (D), or a pharmaceutically acceptable salt thereof with a pharmaceutically-acceptable adjuvant, diluent, carrier or excipient.

41. A process for the preparation of a combination product comprising bringing into association a compound of formula I, as defined in claim 1 without the provisos, or a pharmaceutically acceptable salt thereof with the other therapeutic agent that is useful in the treatment of i) an inflammatory disorder; ii) a disorder in which the modulation of intracellular cyclic adenosine 5′-monophosphate levels within a mammal is desired, which disorder may be an inflammatory disorder; iii) a disorder associated with pathological conditions that are modulated by inhibiting enzymes associated with secondary cellular messengers; iv) transplant rejection in a mammal; v) uncontrolled cellular proliferation; vi) a disorder associated with the central nervous system a and at least one pharmaceutically-acceptable adjuvant, diluent, carrier or excipient.
